(19) United States
(12) Patent Application Publication
(10) Pub. No.: US 2024/0148709 A1
(43) Pub. Date: May 9, 2024

(54) METHODS OF TREATING EYE PAIN AND EYE DISORDERS

(71) Applicant: Aisa Pharma, Inc., Boston, MA (US)

(72) Inventor: Andrew Sternlicht, Boston, MA (US)

(21) Appl. No.: 18/503,371

(22) Filed: Nov. 7, 2023

Related U.S. Application Data
(60) Provisional application No. 63/530,375, filed on Aug. 2, 2023.
(60) Provisional application No. 63/423,678, filed on Nov. 8, 2022.

(51) Int. Cl. A61K 31/4422; A61K 45/06; A61P 9/00; A61P 27/02

(52) U.S. Cl. CPC A61K 31/4422; A61K 45/06; A61P 9/00; A61P 27/02

(57) ABSTRACT

This disclosure relates to methods of treating, e.g., eye pain and eye disorders, using dual N-type and L-type calcium channel blockers selective for the N-type calcium channel (e.g., cilnidipine).

### CROSS-REFERENCE TO RELATED APPLICATION

This application claims priority to U.S. Provisional Application No. 63/530,375, filed on Aug. 2, 2023; and U.S. Provisional Application No. 63/423,678, filed on Nov. 8, 2022, which are incorporated by reference herein in their entirety.

### TECHNICAL FIELD

This disclosure relates to methods of treating, e.g., eye pain and eye disorders, using dual N-type and L-type calcium channel blockers selective for the N-type calcium channel (e.g., cilnidipine).

### BACKGROUND

Greater than 1 billion people worldwide have an eye disease or eye disorder that causes pain or visual impairment. Eye pain can be broadly classified similar to body pain as broadly falling into either nociceptive pain or neuropathic pain. In the latter, normal nervous transmission and functioning is disturbed. Many ocular pain conditions can present with components of either nociceptive or neuropathic pain or both. Eye diseases and disorders that may produce significant ocular pain include, for example, glaucoma, chalazion, dry eye disease, uveitis, optic neuritis, ectropion, entropion, keratitis, scleritis. microvascular cranial nerve palsy, hyphema, post-herpetic neuralgia, post-surgical procedure pain, and post ocular injection procedure pain. Current treatment of ocular pain includes topical steroid application, topical and systemic non-steroidal anti-inflammatory drugs (NSAIDS), gel or eye drops, topical anesthetics, or oral opioids and gabapentanoids. Ocular pain treatments in development include agents that target the vanilloid receptor, TRPV-1 and the sodium channel subunit receptor Nav 1.7. In general, these agents as a group often provide incomplete relief, and have problematic side effects and adverse events. These include the generation of ulcers with topical NSAIDS, corneal anesthesia with risk of injury from foreign bodies with the use of local anesthetics, corneal surface disruption, keratopathy, herpetic keratitis (e.g., from local anesthetic use and constipation), and somnolence from opioid use. A medical need exists for better treatments for ocular pain, particularly for chronic neuropathic ocular pain with improvements in the accompanying side effect profile compared with current treatment options.

### SUMMARY

Described herein are methods and compositions that include the use of dual N-type and L-type calcium channel blockers selective for the N-type calcium channel that can also, in some embodiments, possess selective sodium channel blocker activity (e.g., Nav 1.7 sodium channel blocker activity) and transient receptor potential vanilloid-1 ion channel (TRP-v1) blocker activity, or reduce the release of Interleukin-1 (IL-1), for the treatment of eye pain in a subject.

The cornea is the most highly enervated structure in the body and N-type calcium channels also co-locate on corneal peripheral nerve termini. N-type calcium channels are localized, e.g., at the sympathetic pre-synaptic nerve terminals and play a role in the release of neurotransmitters such as gamma-aminobutyric acid (GABA), acetylcholine, dopamine, and norepinephrine. N-type calcium channels are known to regulate, e.g., neuronal excitability and the firing of action potentials in the neurons, which increases the transmission of neurotransmitters in nociceptive pathways. These neurotransmitters then bind to the receptors on the sensory neurons that cause a person to feel pain. The induction of neuropathic pain can, in certain cases, be a result of the redistribution and alteration of subunit compositions of sodium and calcium channels that can result in spontaneous firing at abnormal locations along the sensory pathway, or a heightened amplified response to what normally might be subthreshold stimuli (hyperalgesia). This may result in unpleasant sensory perceptions including, for example, burning pain, a feeling of wetness, itching, electrical shock pain, and the sensation of pins and needles. Receptors on the surface of the eye generally fall into one of three categories of receptors. Mechano-nociceptors are excited by noxious mechanical stimuli, polymodal heat receptors respond to exogenous irritants, and endogenous inflammatory mediators, and cold receptors respond to moderate temperature changes.

Neuropathic pain is notoriously difficult to treat, with only 40-60% of patients achieving a degree of relief after treatment. Existing drugs have the potential for serious side effects that are, without wishing to be bound by theory, believed to be at least in part the result of unselective (e.g., non-discriminate or low selectivity) calcium channel inhibition. In addition, calcium channel antagonists have some analgesic attributes but have low efficacy in part limited by narrow therapeutic dose ranges as well as insufficient activity at specific relevant ion channel types involved with analgesia.

Based on these considerations, N-selective dual N- and L-type calcium channel inhibition, with increased activity at N-type channels compared with widely used calcium channel antagonists, can be useful to treat diseases and disorders that are associated with dysregulation of blood flow and sympathetic nervous system overactivity, including those featuring symptoms of neuropathic pain. Dual N-type and L-type calcium channel blockers selective for the N-type calcium channel can also inhibit, e.g., the Nav 1.7 sodium channel, and decrease TRPV-1 activity which is increased in neuropathic pain states, and reduce IL-1 production, similarly increased in chronic ocular pain conditions, providing an additional mechanistic pathway contributing to the treatment of neuropathic pain. This can be useful in the treatment of certain conditions and origins for ocular pain, for example, neuropathic ocular pain, such as the pain observed in dry eye disease, which is believed to be caused by corneal nerve dysfunction and overactivity of the Nav 1.7 channels.

Further, it is understood that a cause of ocular pathology and an occasional cause of eye pain in many eye diseases and disorders such as glaucoma is abnormally high intraocular pressure. As such, a number of therapeutic approaches for treating eye pain and eye diseases and disorders (such as the administration of timolol) reduce intraocular pressure. Without wishing to be bound by theory, dual N-type and L-type calcium channel blockers selective for the N-type calcium channel reduce intraocular pressure by (1) reducing blood flow to the ciliary body through arteriole dilation, which leads to a decrease in the production of aqueous humor, (2) reducing cAMP levels in the eye, and/or (3) acting on the trabecular network to improve permeability and emptying. As such, dual N-type and L-type calcium channel blockers selective for the N-type calcium channel have the potential to treat eye pain by, for example, ameliorating neuropathic eye pain and reducing intraocular pressure.

Further, blockade of N-channels increases perfusion to the optic nerve and thus has a beneficial effect on retinal ganglion and amacrine cells, where the N-channels are located. Accordingly, dual N-type and L-type calcium channel blockers selective for the N-type calcium channel may have a neuroprotective action to slow cell loss associated with disease or actually restore loss of vision in, e.g., glaucoma.

Further, in order for an agent to have a possible neuroprotective effect on retinal ganglion cells if administered as a topical agent such as eyedrops, in some embodiments it is helpful that such an agent achieve adequate retinal concentrations so as to have these benefits and to penetrate the blood retinal barrier, which is problematic for certain peptide moieties and some larger molecules and some hydrophilic compounds.

Further, it is understood that nociceptors on the surface of the eye relay information to the trigeminal ganglion where pain is modulated with input from both peripheral and central processing. Nociceptive pain involves mechanoreceptors on the surface of the eye and there are separate receptors for heat and cold sensitivity and hyperalgesia. In response to inflammatory stimuli or chronic noxious stimuli, receptor populations can be upregulated and their processing changed so that pain becomes neuropathic in nature with hyperalgesia and sensitivity. Thermal hyperalgesia is in turn modulated though different mediators with TRPV-1 involved in heat hyperalgesia and pain modulation stemming from alterations in tear fluid, and cold thermoreceptors, also expressed on the eye surface in abundance show increased expression of TRPM-8, the melanin cortropin product. Cold receptors play a dominant role in the perception of pain associated with dry eye disease. Both heat and cold receptors contribute to various ocular pain syndromes and both pathways are affected by voltage gated calcium channel pathways, specifically N-channel activity but L channel activity has been correlated with TRMP8 expression as well. In oxyplatin-induced neuropathy, TRMP8 expression is increased but patients taking L-channel dihydropyridine antagonists don't show this increased expression and have less neuropathy. The dual N-type and L-type calcium channel blocker selective for the N-type calcium channel cilnidipine has been shown to reduce the cold pressor stress response when hands are placed into cold water (4° C.), with the blood pressure response mediated by cold thermoreceptors in the skin. Without wishing to be bound by theory, it is believed that a similar attenuation of cold receptor activity can occur in ocular cold thermoreceptors on the surface of the eye which will thereby reduce ocular pain in certain conditions, including but not limited to dry eye disease, in which these cold receptors mediate pain transmission and perception. Normal or physiological pain results from the stimulation by noxious stimuli of sensory axons of trigeminal ganglion (TG) neurons innervating the eye. These are functionally heterogeneous. Mechano-nociceptors are only excited by noxious mechanical forces. Polymodal nociceptors also respond to heat, exogenous irritants, and endogenous inflammatory mediators, whereas cold thermoreceptors detect moderate temperature changes. Their distinct sensitivity to stimulating forces is determined by the expression of specific classes of ion channels: Piezo2 for mechanical forces, TRPV1 and TRPA1 for heat and chemical agents, and TRPM8 for cold. Pricking pain is evoked by mechano-nociceptors, while polymodal nociceptors are responsible of burning and stinging eye pain; sensations of dryness appear to be mainly evoked by cold thermoreceptors. Mediators released by local inflammation increase the excitability of eye polymodal nociceptors causing their sensitization and the augmented pain sensations. During chronic inflammation, additional, long-lasting changes in the expression and function of stimulus transducing and voltage-sensitive ion channels develop, thereby altering polymodal terminal's excitability and evoking chronic inflammatory pain. When trauma, infections, or metabolic processes directly damage eye nerve terminals, these display aberrant impulse firing due to an abnormal expression of transducing and excitability modulating ion channels. This malfunction evokes ‘neuropathic pain’ which may also result from abnormal function of higher brain structures where ocular TG neurons project. Eye diseases or ocular surface surgery cause different levels of inflammation and/or nerve injury, which in turn activate sensory fibers of the eye in a variable degree. When inflammation dominates (allergic or actinic kerato-conjunctivitis), polymodal nociceptors are primarily stimulated and sensitized, causing pain. In uncomplicated photorefractive surgery and moderate dry eye, cold thermoreceptors appear to be mainly affected, evoking predominan sensations of unpleasant dryness.

Another beneficial effect of L-type calcium channel inhibition is the dilation of the arteries in smooth muscle, causing an increase in arterial diameter, referred to as vasodilation. However, L-type calcium channel inhibition induces a homeostatic reflex mechanism in which norepinephrine is produced. The norepinephrine induces vasoconstriction, thus partially offsetting the vasodilating effects of the L-type calcium channel inhibition. A useful complementary effect of N-type calcium channel inhibition is the decrease of norepinephrine release and sympathetic outflow pre-synaptically in the spinal cord at the level of the dorsal root ganglion, which can counteract the homeostasis mechanism triggered by blockade of the L-type calcium channel. Disclosed herein are dual N-type and L-type calcium channel blockers selective for the N-type calcium channel (e.g., from about 5-fold to 50-fold to about 100-fold selective) which can, for example, (1) reduce neuropathic pain, (2) induce vasodilation, and (3) counter the homeostatic vasoconstriction triggered by blockade of the L-type calcium channel.

Selective inhibition of the N-type calcium channel has been shown to result in reduced severity and/or frequency of side effects and increased tolerability compared to non-N-selective calcium channel blockade. Further, dual N-type and L-type calcium channel blockers selective for the N-type calcium channel may be effective for certain conditions at lower dosages and may provide higher efficacy relative to less selective calcium channel blockers at equipotent doses as regards their blood pressure lowering effects in hypertensive individuals.

Additional advantages of selective N-channel blockade by dual N-type and L-type calcium channel blockers selective for the N-type calcium channel, compared to calcium channel blockers that lack N-channel selectivity, can include:
Ability to dose locally (topically) as well as orally for systemic activity to affect both peripheral and central sites of pain generation/transmission.
Unique physicochemical properties of small molecule topical application allows better access to pain generator targets on cornea surface, nerve site receptors, optic nerve processing sites, and diffusion/distribution to more sites of biologic pain generators (as opposed to peptides).
Broader applicability to treat most neuropathic ocular pain conditions than specific TRPV-1 or Nav 1.7 inhibitors since activity occurs at both pathways.
In some embodiments, lipophilic formulation of topical drops is possible, which may improve tear preservation/moistness in dry eye disease as opposed to possible other aqueous formulations of other agents.
Broad applicability across many pain conditions, including, but not limited to, dry eye disease, glaucoma, uveitis, inflammation, Post-herpetic neuralgia with eye involvement, Allergies, Blepharitis, Chalazion, complication of eye surgery (like PRK), Contact lens problems, Corneal abrasion, Ectropion, Entropion, Eyelid infection, Eye injection of various therapeutic agents, Foreign object in the eye, Glaucoma, Injury, Iritis, Keratitis, Optic neuritis, Pink eye (conjunctivitis), Scleritis Stye (sty).
Neuroprotective effect of dual N-type and L-type calcium channel blockers selective for the N-type calcium channel (e.g., cilnidipine) may have effects in glaucoma beyond reduction of pain.
Neuroprotective effect of dual N-type and L-type calcium channel blockers selective for the N-type calcium channel (e.g., cilnidipine) may have benefit in reduction of geographic atrophy in age related macular degeneration.
Improvement in endothelial function and endothelial concentrations of nitric oxide by improving blood flow, reducing pain that is, e.g., a consequence of reduced blood flow.
Improvement in cardiac and left ventricle functioning resulting in reduction of pain due, e.g., to ischemia.
Improvement in the incidence and severity of atherosclerosis including reducing pain caused, e.g., by a reduction in blood flow, and reducing the overall incidence of atherosclerotic-related events.
Decrease in overall sympathetic nervous system activity and plasma concentration of norepinephrine, which can decrease pain due to net arteriole dilation and decrease in sympathetically mediated pain syndromes.
Improvement in overall autonomic functioning, which may improve gut function in patients whose gut function (e.g., competency of the lower esophageal sphincter and peristalsis and gastric emptying), is compromised due to impaired autonomic function as occurs in certain disease states (e.g., scleroderma).
Decrease in oxidative stress and reduction in reactive oxygen species.

Dual N-type and L-type calcium channel blockers selective for the N-type calcium channel, such as cilnidipine, are also understood to have activity at the Nav 1.7 sodium channel, and likely have activity at the TRP-v1 receptor (i.e., the capsaicin receptor) as has been shown with N-channel antagonists. Nav 1.7 channels are expressed in the nociceptive neurons at dorsal root ganglion, geminal ganglion, and sympathetic ganglion neurons, which are part of the autonomic nervous system and mediate pain, thus providing cilnidipine an additional mode of action for alleviating pain. It has been demonstrated that a 20 mg dose of cilnidipine provides >6-fold inhibition of Nav 1.7 compared to an 1800 mg dose of gabapentin. See, for example, published PCT application WO2021/178903 which is incorporated by reference herein in its entirety. The TRP-v1 receptor is located at peripheral nociceptors and mediates conditions characterized by thermal hypersensitivity (e.g., thermal allodynia and thermal hyperalgesia).

Other aspects of the invention will become apparent by consideration of, e.g., the detailed description and claims.

### Definitions

As used herein, the terms “about” and “approximately” are used interchangeably, and when used to refer to modify a numerical value, encompass a range of uncertainty of the numerical value of from 0% to 10% of the numerical value.

As used herein, the singular forms “a,” “an,” and “the” include plural referents unless the context clearly dictates otherwise.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the disease or disorder is associated with dysregulation of blood flow and sympathetic nervous system overactivity. In some embodiments, the disease or disorder is characterized by neuropathic pain, vasoconstriction, dysesthetic pain, burning pain, body temperature changes of the subject, hyperesthesia, changes in skin or tissue color, edema, changes in skin turgor, ruble, pallor, cyanosis, vasospasm, or any combination thereof.

As used herein, the phrase “fixed dosage form” refers to the simultaneous administration of a dual N-type and L-type calcium channel blocker selective for the N-type calcium channel and at least one additional therapeutic agent (e.g., a beta blocker such as timolol) to a subject in a single dosage form (e.g., a topical solution).

As used herein, the term “dual N-type and L-type calcium channel blocker selective for the N-type calcium channel”, “selective N-type calcium channel blocker”, “selective N-type CCB”, and “N-type selective CCB” refer to an agent that inhibits both N- and L-type calcium channels, and inhibits the N-type calcium channel to a greater degree than the L-type calcium channel. In some embodiments, the dual N-type and L-type calcium channel blocker selective for the N-type calcium channel has at least a 5-fold selectivity for the N-type calcium channel over the L-type calcium channel. For example, the dual N-type and L-type calcium channel blocker selective for the N-type calcium channel exhibits at least a 10-fold, at least a 30-fold, at least a 50-fold, at least a 80-fold, at least a 100-fold, at least a 300-fold, at least a 500-fold, at least a 800-fold, at least a 900-fold, or at least a 1000-fold selectivity for the N-type calcium channel over the L-type calcium channel. For example, the dual N-type and L-type calcium channel blocker selective for the N-type calcium channel exhibits at least a 50-fold to 100-fold selectivity for the N-type calcium channel over the L-type calcium channel. In some embodiments, the dual N-type and L-type calcium channel blocker selective for the N-type calcium channel exhibits a greater selectivity for the N-type calcium channel over the L-type calcium channel than a non-N-selective calcium channel blocker. In some embodiments, the non-N-selective calcium channel blocker is amlodipine, nifedipine, nicardipine, nimodipine, diltiazem or verapamil. In some embodiments, the dual N-type and L-type calcium channel blocker selective for the N-type calcium channel exhibits at least a 5% (e.g., at least a 10%, 20%, 30%, 40%, 50%, 60%, 70%, 80%, 100%, 150%, 200%, 300%) greater selectivity for the N-type calcium channel over the L-type calcium channel than a non-N-selective calcium channel blocker. Examples of dual N-type and L-type calcium channel blocker selective for the N-type calcium channel include, but are not limited to, cilnidipine, Z-160, ralfinamide, CNV2197944, or pharmaceutically acceptable salts thereof. Examples of dual N-type and L-type calcium channel blocker selective for the N-type calcium channel include, but are not limited to, cilnidipine, Z-160, CNV2197944, or pharmaceutically acceptable salts thereof. Examples of dual N-type and L-type calcium channel blocker selective for the N-type calcium channel include, but are not limited to, cilnidipine, Z-160, zicinotide, and pharmaceutically acceptable salts thereof.

In some embodiments, the dual N-type and L-type calcium channel blocker selective for the N-type calcium channel is also a Nav 1.7 sodium channel blocker (i.e., the dual N-type and L-type calcium channel blocker selective for the N-type calcium channel further inhibits a Nav 1.7 sodium channel). In some embodiments, the dual N-type and L-type calcium channel blocker selective for the N-type calcium channel that further inhibits a sodium channel (e.g., Nav 1.7) is cilnidipine. In some embodiments, the dual N-type and L-type calcium channel blocker selective for the N-type calcium channel is also a TRP-v1 channel inhibitor (i.e., the dual N-type and L-type calcium channel blocker selective for the N-type calcium channel further inhibits a TRP-v1 channel). In some embodiments, the dual N-type and L-type calcium channel blocker selective for the N-type calcium channel that further inhibits a TRP-v1 channel inhibitor is cilnidipine.

As used herein, the terms “non-N-type selective calcium channel blocker” and “non-N-selective calcium channel blocker” refer to an agent that blocks one or more calcium channels, but either (1) does not block the N-type calcium channel, or (2) blocks the N-type calcium channel, but not selectively over the L-type calcium channel. Examples of non-N-type selective calcium channel blockers include, but are not limited to, nifedipine, nicardipine, amlodipine, Z-944, nimodipine, verapamil, diltiazem, felodipine, isradipine, nisoldipine, mibafredil, nilvidipine barnidipine, benidipine lacidipine, lercanidipine, manidipine and nitrendipine, and pharmaceutical salts thereof.

As used herein, the term “Nav 1.7 sodium channel blocker” or “Nav 1.7 sodium channel inhibitor” refers to an agent that can inhibit the Nav 1.7 sodium channel. In some embodiments, the Nav 1.7 sodium channel blocker inhibits the closed state of the Nav 1.7 sodium channel. In some embodiments, the Nav 1.7 sodium channel blocker inhibits the inactivated state of the Nav 1.7 sodium channel. In some embodiments, the Nav 1.7 sodium channel blocker is also a dual N-type and L-type calcium channel blocker selective for the N-type calcium channel (i.e., the Nav 1.7 sodium channel blocker further inhibits the N-type and L-type calcium channels selectively for the N-type calcium channel). In some embodiments, the Nav 1.7 sodium channel blocker is also a TRP-v1 inhibitor (i.e., the Nav 1.7 sodium channel blocker further inhibits a TRP-v1 channel). In some embodiments, the Nav 1.7 sodium channel blocker that further inhibits the N-type and L-type calcium channels selectively for the N-type calcium channel is cilnidipine.

As used herein, the term “TRP-v1 inhibitor” refers to an agent that can inhibit the TRP-v1 channel. In some embodiments, the TRP-v1 inhibitor is also a dual N-type and L-type calcium channel blocker selective for the N-type calcium channel (i.e., the TRP-v1 inhibitor further inhibits the N-type and L-type calcium channels selectively for the N-type calcium channel). In some embodiments, the TRP-v1 inhibitor is also a Nav 1.7 sodium channel blocker (i.e., the TRP-v1 inhibitor further inhibits the Nav 1.7 sodium channel).

As used herein, the term “adverse effect” refers to an undesirable effect resulting from an alteration in normal physiology in a subject.

As used herein, the term “vasoconstriction” refers to the reduction in diameter of a blood vessel (e.g., an artery, vein, or capillary) resulting in reduced blood flow to the tissue the vasoconstricted blood vessels circulate blood to and from.

As used herein, the term “body temperature” refers to the temperature range of the body and/or one or more parts of the body in a healthy, awake subject under normal conditions of thermoregulation as measured, for example, in the mouth, the rectum, the armpit, the hands, the feet, or the ear. For example, the temperature range in a healthy human subject under normal conditions of thermoregulation in the rectum, heart, oropharynx, tympanic membrane, and esophagus is 36.1° C. to 37.8° C.; and the temperature range in a healthy human subject under normal conditions of thermoregulation in the hand or foot (e.g., hand) can be 18° C. to 37.8° C., depending on the ambient temperature.

The term “therapeutically effective amount,” as used herein, refers to a sufficient amount of a chemical entity (e.g., a dual N-type and L-type calcium channel blocker selective for the N-type calcium channel) being administered which will relieve to an extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: transdermal, intranasally, rectal, inhalational, subcutaneous, intradermal, sublingual, intraspinal, epidural, or ocular administration.

The term “abnormally” when used, for example, in the terms “abnormally high”, “abnormally elevated”, or “abnormally low”, means a deviation from the range of the parameter being referred to that is found in a healthy subject as would be recognized by a medical professional, and that can be considered as indicative or predictive of dysfunction or a pathological state. Further, “abnormal” can, in some embodiments, refer to a physiologic response that is persistent beyond when a normal person would have recovered from that response; or a physiologic response that is exaggerated in degree and/or duration relative to what occurs in a normal, healthy subject. In some embodiments, the medical professional is, for example, an ophthalmologist, an optometrist, an orthoptist, an optician, an ocularist, a physician, a nurse practitioner, a physician assistant, a nurse, or a medical laboratory scientist.

As used herein, the phrase “analgesia of the eye” refers to pain relief of the eye.

As used herein, the phrase “anesthesia of the eye” refers to numbing of the eye.

For purposes of clarification, unless otherwise specified herein, when a variable (e.g., condition, feature, state, parameter, score, or statistic) in a subject is increased, decreased, or improved, the increase, decrease, or improvement is, for example, measured, assessed, or obtained in relation to the same variable measured, assessed, or obtained before the start of treatment (e.g., before administration of the dual N-type and L-type calcium channel blocker selective for the N-type calcium channel), unless otherwise specified herein. The variable can be a single measurement, assessment, or score; an average of a plurality of measurements, assessments, or scores; or a daily average of a plurality of measurements, scores, or assessments. Unless otherwise specified herein, measurements, assessments, or scores are typically taken within 1 month (e.g., within 3 weeks, 2 weeks, 10 days, 9 days, 8 days, 7 days, 6 days, 5 days, 4 days, 3 days, 2 days, 1 day, 18 hours, 12 hours, or 6 hours, 3, 2, 1, ½, ¼ hours) of the administration of the dual N-type and L-type calcium channel blocker selective for the N-type calcium channel. For example, reducing the frequency of symptoms in a subject can occur, e.g., when the number or average number of symptomatic episodes perceived by the subject that occurred during a span of time after administration of the dual N-type and L-type calcium channel blocker selective for the N-type calcium channel is less than the number or average number of symptomatic episodes perceived by the subject that occurred during the same span of time before administration of the dual N-type and L-type calcium channel blocker selective for the N-type calcium channel.

The details of one or more embodiments of the invention are set forth in the description below. Other features and advantages of the invention will be apparent from the description and drawings, and from the claims.

### DESCRIPTION OF DRAWINGS

FIG. 1 shows the schematic of the study of Example 2.

FIG. 2 shows a schedule of assessments in the double-blind parallel group.

FIG. 3A and FIG. 3B show a schedule of assessments in the double-blind 2-way crossover group.

### DETAILED DESCRIPTION

Herein is disclosed a method of treating eye pain in a subject in need thereof, the method comprising administering a therapeutically effective amount of a dual N-type and L-type calcium channel blocker selective for the N-type calcium channel to an eye of the subject. In some embodiments, the pain is neuropathic pain.

Examples of N-type calcium channels include, but are not limited to, the Cav 2.2 Type, which has two subunits, Cav 2.2a and Cav2.2b, both of which have an alpha 1 subunit of 2.2 and are affected by N type current.

Herein is disclosed a method of treating an eye disease or disorder in a subject in need thereof, the method comprising administering a therapeutically effective amount of a dual N-type and L-type calcium channel blocker selective for the N-type calcium channel to an eye of the subject, wherein the disease or disorder is selected from allergies, blepharitis, chalazion, complication of eye surgery, contact lens problem, corneal abrasion, corneal herpetic infections (herpes), dry eyes, dry eye disease, ectropion, entropion eyelid infection, foreign object in the eye, glaucoma, hyphema, injury (e.g., mechanical injury) to the eye, iritis, keratitis, microvascular cranial nerve palsy, optic neuritis, pink eye (conjunctivitis), scleritis, or stye (sty).

In some embodiments, the disease or disorder is selected from dry eye disease, uveitis, optic neuritis, microvascular cranial nerve palsy, hyphema, and glaucoma.

In some embodiments, the disease or disorder is dry eye disease.

In some embodiments, the disease or disorder is uveitis.

In some embodiments, the disease or disorder is optic neuritis.

In some embodiments, the disease or disorder is microvascular cranial nerve palsy.

In some embodiments, the disease or disorder is hyphema.

In some embodiments, the disease or disorder is glaucoma.

Herein is disclosed a method of treating dry eye disease in a subject in need thereof, the method comprising administering a therapeutically effective amount of a dual N-type and L-type calcium channel blocker selective for the N-type calcium channel to an eye of the subject.

Herein is disclosed a method of treating glaucoma in a subject in need thereof, the method comprising administering a therapeutically effective amount of a dual N-type and L-type calcium channel blocker selective for the N-type calcium channel to an eye of the subject.

In some embodiments, the glaucoma is selected from the group consisting of: open-angle glaucoma, angle-closure glaucoma, normal-tension glaucoma, congenital glaucoma, or a secondary glaucoma. In some embodiments, the glaucoma is open-angle glaucoma. In some embodiments, the glaucoma is angle-closure glaucoma. In some embodiments, the glaucoma is normal-tension glaucoma. In some embodiments, the glaucoma is congenital glaucoma. In some embodiments, the glaucoma is a secondary glaucoma. In some embodiments, the secondary glaucoma is selected from the group consisting of: neovascular glaucoma, pigmentary glaucoma, exfoliation glaucoma, and uveitic glaucoma. In some embodiments, the secondary glaucoma is neovascular glaucoma. In some embodiments, the secondary glaucoma is pigmentary glaucoma. In some embodiments, the secondary glaucoma is exfoliation glaucoma. In some embodiments, the secondary glaucoma is uveitic glaucoma.

Herein is disclosed a method of treating intraocular hypertension in a subject in need thereof, the method comprising administering a therapeutically effective amount of a dual N-type and L-type calcium channel blocker selective for the N-type calcium channel to an eye of the subject.

Without wishing to be bound by theory, it is believed that the dual N-type and L-type calcium channel blocker selective for the N-type calcium channel reduces intraocular pressure by (1) reducing blood flow to the ciliary body through arteriole dilation, which leads to a decrease in the production of aqueous humor, (2) reducing cAMP levels in the eye, and/or (3) acts on the trabecular network to improve permeability and emptying.

In some embodiments, after administering the dual N-type and L-type calcium channel blocker selective for the N-type calcium channel, intraocular pressure in an eye of the subject is reduced compared to the intraocular pressure in the eye before administering the dual N-type and L-type calcium channel blocker selective for the N-type calcium channel.

Herein is disclosed a method of reducing intraocular pressure in an eye of a subject in need thereof, the method comprising:

administering a therapeutically effective amount of a dual N-type and L-type calcium channel blocker selective for the N-type calcium channel to the eye of the subject, and assessing or measuring a reduction in the intraocular pressure in the eye of the subject, wherein the reduction is assessed or measured in comparison to the intraocular pressure in the eye before administering the dual N-type and L-type calcium channel blocker selective for the N-type calcium channel.

Herein is disclosed a method of reducing intraocular pressure in an eye of a subject in need thereof, the method comprising:
selecting a subject with an intraocular pressure recognized by a medical professional as abnormally high;
administering a therapeutically effective amount of a dual N-type and L-type calcium channel blocker selective for the N-type calcium channel to the eye of the subject, and
assessing or measuring a reduction in the intraocular pressure in the eye of the subject, wherein the reduction is assessed or measured in comparison to the intraocular pressure in the eye before administering the dual N-type and L-type calcium channel blocker selective for the N-type calcium channel.

In some embodiments, selecting a subject with an intraocular pressure recognized by a medical professional as abnormally high (e.g., above 15 mm Hg, above 17 mm Hg, above 19 mm Hg, or above 21 mm Hg) comprises measuring the intraocular pressure of an eye of the subject by applanation tonometry. In some embodiments, the reduction in the intraocular pressure in the eye of the subject is measured by applanation tonometry (i.e., ocular tonometry). More information on applanation tonometry can be found in (1) Palay, David A.; Krachmer, J. H. Primary Care Ophthalmology, 2005 (see https://doi.org/10.1016/B978-0-323-03316-9.X5001-3); and (2) https://en.wikipedia.org/wiki/Ocular tonometry, both of which are incorporated by reference herein in their entirety.

In some embodiments, a reduction in the intraocular pressure in the eye of the subject of at least 1% (e.g., at least 2%, at least 5%, at least 10%, at least 15%, at least 20%, at least 30%, at least 40%, at least 50%, at least 60%, at least 75%, at least 90%) is measured by applanation tonometry after administering the dual N-type and L-type calcium channel blocker selective for the N-type calcium channel to the subject. For example, a reduction in the intraocular pressure in the eye of the subject of at least 2% is measured by applanation tonometry. For example, a reduction in the intraocular pressure in the eye of the subject of at least 10% is measured by applanation tonometry.

In some embodiments, a reduction in the intraocular pressure in the eye of the subject of at least 1 mm Hg (e.g., at least 2 mm Hg, at least 3 mm Hg, at least 4 mm Hg, at least 5 mm Hg, at least 7 mm Hg, at least 12 mm Hg, at least 15 mm Hg, at least 20 mm Hg, at least 25 mm Hg, at least 30 mm Hg, at least 40 mm Hg) is measured by applanation tonometry after administering the dual N-type and L-type calcium channel blocker selective for the N-type calcium channel to the subject. For example, a reduction in the intraocular pressure in the eye of the subject of at least 2 mm Hg is measured by applanation tonometry. For example, a reduction in the intraocular pressure in the eye of the subject of at least 10 mm Hg is measured by applanation tonometry.

In some embodiments, the intraocular pressure in the eye of the subject is reduced to about 15 mm Hg to about 17 mm Hg. In some embodiments, the intraocular pressure in the eye of the subject is reduced to about 12 mm Hg to about 15 mm Hg. In some embodiments, the intraocular pressure in the eye of the subject is reduced to about 10 mm Hg to about 12 mm Hg.

In some embodiments, the subject is identified or diagnosed as having eye pain or an eye disease or disorder. In some embodiments, the subject is identified or diagnosed as having an intraocular pressure that is recognized by a medical professional as abnormally high. In some embodiments, an abnormally high intraocular pressure is at least about 22 mm Hg, for example, at least about 24 mm Hg, at least about 26 mm Hg, at least about 28 mm Hg, or at least about 30 mm Hg.

In some embodiments, reducing intraocular pressure comprises reducing blood pressure to the ciliary body, reducing the volume of aqueous humor in the eye, dilating the trabecular network in the eye, increasing outflow of aqueous humor from the anterior chamber of the eye, or any combination thereof.

In some embodiments, an optic nerve of the subject is recognized or identified as having reduced functioning in relation to a normal or healthy optic nerve as determined by a medical professional. In some embodiments, functioning of the optic nerve of the subject is improved after administering the dual N-type and L-type calcium channel blocker selective for the N-type calcium channel. In some embodiments, a retinal ganglion cell and/or a starburst amacrine cell is recognized or identified as having reduced functioning in relation to a corresponding normal or healthy cell as determined by a medical professional. In some embodiments, functioning of the retinal gangion cell and/or starburst amacrine cell of the subject is improved after administering the dual N-type and L-type calcium channel blocker selective for the N-type calcium channel. Without wishing to be bound by theory, the dual N-type and L-type calcium channel blocker selective for the N-type calcium channel is believed to improve the functioning of optic nerves, retinal ganglion cells, and/or starburst amacrine cells reducing ischemia reperfusion injury.

In some embodiments, the subject is diagnosed with an eye disease or disorder selected from the group consisting of: allergies, blepharitis, chalazion, complication of eye surgery, contact lens problem, corneal abrasion, corneal herpetic infections (herpes), dry eyes, dry eye disease, ectropion, entropion eyelid infection, foreign object in the eye, glaucoma, hyphema, injury (e.g., mechanical injury) to the eye, iritis, keratitis, microvascular cranial nerve palsy, optic neuritis, pink eye (conjunctivitis), scleritis, or stye (sty). In some embodiments, the subject is diagnosed with an eye disease or disorder selected from the group consisting of: dry eyes and dry eye disease, uveitis, optic neuritis, microvascular cranial nerve palsy, hyphema, glaucoma, or any combination thereof. In some embodiments, the disease or disorder is dry eye disease. In some embodiments, the disease or disorder is uveitis. In some embodiments, the disease or disorder is optic neuritis. In some embodiments, the disease or disorder is microvascular cranial nerve palsy. In some embodiments, the disease or disorder is hyphema. In some embodiments, the glaucoma is selected from the group consisting of: open-angle glaucoma, angle-closure glaucoma, normal-tension glaucoma, congenital glaucoma, or a secondary glaucoma. In some embodiments, the glaucoma is open-angle glaucoma. In some embodiments, the glaucoma is angle-closure glaucoma. In some embodiments, the glaucoma is normal-tension glaucoma. In some embodiments, the glaucoma is congenital glaucoma. In some embodiments, the glaucoma is a secondary glaucoma. In some embodiments, the secondary glaucoma is selected from the group consisting of: neovascular glaucoma, pigmentary glaucoma, exfoliation glaucoma, and uveitic glaucoma. In some embodiments, the secondary glaucoma is neovascular glaucoma. In some embodiments, the secondary glaucoma is pigmentary glaucoma. In some embodiments, the secondary glaucoma is exfoliation glaucoma. In some embodiments, the secondary glaucoma is uveitic glaucoma.

In some embodiments, the subject has pain in an eye, and wherein after administering the dual N-type and L-type calcium channel blocker selective for the N-type calcium channel, the eye pain is reduced. In some embodiments, the pain is neuropathic pain. In some embodiments, the reduction in eye pain is assessed or measured by the eye-wiping test, the Ocular Pain Assessment Scale (OPAS), the Schirmer Tear Test (STT), applanation tonometry, or the ocular surface disease index (OSDI).

In some embodiments, the reduction in eye pain is assessed or measured by the eye-wiping test. In some embodiments, at least 1 (e.g., 1, 2, 3, or 4) fewer eye wipes are observed during a 30 second time period than before administration of the dual N-type and L-type calcium channel blocker selective for the N-type calcium channel. More information on the eye wiping test can be found in Brain Research Protocols, 2006, 16(1-3), 44-49, which is incorporated by reference herein in its entirety.

In some embodiments, the reduction in eye pain is assessed or measured by the Ocular Pain Assessment Survey (OPAS). In some embodiments, the reduction in eye pain is assessed by a reduction in the overall severity score (OPAS question 1), a reduction in the combined eye pain intensity 24 hours score (OPAS questions 4, 5, and 6), and/or a reduction in the combined eye pain intensity score (OPAS questions 7, 8, and 9). In some of these embodiments, a reduction is an at least 1 point (e.g., at least 2 point, at least 3 point, at least 4 point, at least 5 point, at least 7 point, 1 point, 2 point, 3 point, 4 point, 5 point, 6 point, or 7 point) reduction. In some embodiments, after administering the dual N-type and L-type calcium channel blocker selective for the N-type calcium channel, a reduction in the overall severity score (OPAS question 1), a reduction in the combined eye pain intensity 24 hours score (OPAS questions 4, 5, and 6), and/or a reduction in the combined eye pain intensity score (OPAS questions 7, 8, and 9) is observed in the subject than before administration of the dual N-type and L-type calcium channel blocker selective for the N-type calcium channel. In some of these embodiments, a reduction is an at least 1 point (e.g., at least 2 point, at least 3 point, at least 4 point, at least 5 point, at least 7 point, 1 point, 2 point, 3 point, 4 point, 5 point, 6 point, or 7 point) reduction. More information on the Ocular Pain Assessment Survey (OPAS) can be found in Ophthalmology, 2016, 123(7), which is incorporated by reference herein in its entirety.

In some embodiments, the reduction in eye pain is assessed or measured by the Schirmer Tear Test (STT). In some embodiments, the rate of travel of the tear fluid in the test strip is at least 2% (e.g., at least 4%, at least 6%, at least 10%, at least 15%, at least 20%, at least 25%, at least 35%, at least 50%, at least 65%, at least 80%, or at least 90%) lower after administration of the dual N-type and L-type calcium channel blocker selective for the N-type calcium channel relative to before administration of the dual N-type and L-type calcium channel blocker selective for the N-type calcium channel. In some embodiments, the rate of travel of the tear fluid in the test strip is at least 2% (e.g., at least 4%, at least 6%, at least 10%, at least 15%, at least 20%, at least 25%, at least 35%, at least 50%, at least 65%, at least 80%, or at least 90%) faster after administration of the dual N-type and L-type calcium channel blocker selective for the N-type calcium channel relative to before administration of the dual N-type and L-type calcium channel blocker selective for the N-type calcium channel. More information on the Schirmer Tear Test (STT) can be found at https://en.wikipedia.org/wiki/Schirmer %27s_test, which is incorporated by reference herein in its entirety.

In some embodiments, the reduction in eye pain is assessed or measured by applanation tonometry. In some embodiments, a reduction in the intraocular pressure in the eye of the subject of at least 1% (e.g., at least 2%, at least 5%, at least 10%, at least 15%, at least 20%, at least 30%, at least 40%, at least 50%, at least 60%, at least 75%, at least 90%) is measured by applanation tonometry after administration of the dual N-type and L-type calcium channel blocker selective for the N-type calcium channel than before administration of the dual N-type and L-type calcium channel blocker selective for the N-type calcium channel. For example, a reduction in the intraocular pressure in the eye of the subject of at least 2% is measured by applanation tonometry. For example, a reduction in the intraocular pressure in the eye of the subject of at least 10% is measured by applanation tonometry. In some embodiments, a reduction in the intraocular pressure in the eye of the subject of at least 1 mm Hg (e.g., at least 2 mm Hg, at least 3 mm Hg, at least 4 mm Hg, at least 5 mm Hg, at least 7 mm Hg, at least 12 mm Hg, at least 15 mm Hg, at least 20 mm Hg, at least 25 mm Hg, at least 30 mm Hg, at least 40 mm Hg) is measured by applanation tonometry. For example, a reduction in the intraocular pressure in the eye of the subject of at least 2 mm Hg is measured by applanation tonometry. For example, a reduction in the intraocular pressure in the eye of the subject of at least 10 mm Hg is measured by applanation tonometry.

In some embodiments, the reduction in eye pain is assessed or measured by the Ocular Surface Disease Index (OSDI). In some embodiments, the reduction in eye pain is assessed by a reduction in the index. In some embodiments, a reduction in the index is assessed after administration of the dual N-type and L-type calcium channel blocker selective for the N-type calcium channel relative to before administration of the dual N-type and L-type calcium channel blocker selective for the N-type calcium channel In some embodiments, the reduction in eye pain comprises an at least 1 point (e.g., at least 2 point, at least 5 point, at least 10 point, at least 15 point, at least 20 point, at least 25 point, at least 30 point, at least 40 point, at least 50 point, at least 60 point, at least 70 point, at least 80 point, or at least 90 point) reduction in the index. In some embodiments, the reduction in eye pain is assessed by a reduction in the severity scale. In some embodiments, the reduction in eye pain is assessed by a reduction of the severity score from severe disease to moderate disease, moderate disease to mild disease, mild disease to none, severe disease to mild disease, severe disease to none, or moderate disease to none.

In some embodiments, the subject has an allergy, blepharitis, chalazion, contact lens problem, corneal abrasion, a herpetic infection (e.g., herpes), dry eyes, dry eye disease, ectropion, entropion, eyelid infection, foreign object in the eye, glaucoma, injury, iritis, keratitis, optic neuritis, pink eye (conjunctivitis), scleritis, stye, uveitis, eye trauma, or any combination thereof. In some embodiments, surgery was performed on the eye of the subject.

In some embodiments, the eye pain is associated with an allergy, blepharitis, chalazion, eye surgery, contact lens problem, corneal abrasion, a herpetic infection (e.g., herpes), dry eyes, dry eye disease, ectropion, entropion, eyelid infection, foreign object in the eye, glaucoma, injury, iritis, keratitis, optic neuritis, pink eye (conjunctivitis), scleritis, stye, uveitis, eye trauma, or any combination thereof.

In some embodiments, an at least 1% (e.g., at least 2%, at least 5%, at least 10%, at least 15%, at least 20%, at least 30%, at least 40%, at least 50%, at least 60%, at least 75%, at least 90%) reduction of intraocular pressure in the eye of the subject is measured by applanation tonometry after administering the dual N-type and L-type calcium channel blocker selective for the N-type calcium channel. For example, an at least 2% reduction of intraocular pressure in the eye of the subject is measured by applanation tonometry after administering the dual N-type and L-type calcium channel blocker selective for the N-type calcium channel. For example, an at least 10% reduction of intraocular pressure in the eye of the subject is measured by applanation tonometry after administering the dual N-type and L-type calcium channel blocker selective for the N-type calcium channel.

In some embodiments, after administration of the dual N-type and L-type calcium channel blocker selective for the N-type calcium channel to the subject, substantially no anesthesia of the eye (e.g., the cornea) is detected in the subject according to the blink reflex test. Anesthesia of the eye is assessed using the blink reflex test. See, for example, Jones et al. The Journal of Headache and Pain (2016) 17:96, and https://www.physio-pedia.com/Blink_Reflex, each of which is incorporated by reference herein in its entirety.

In some embodiments, after administration of the dual N-type and L-type calcium channel blocker selective for the N-type calcium channel to the subject, the visual acuity of the subject does not decrease. In some embodiments, the visual acuity of the subject not decreasing is assessed by a lack of change in or an improvement in a ETDRS letter chart assessment. In some embodiments, after administration of the dual N-type and L-type calcium channel blocker selective for the N-type calcium channel to the subject, the visual acuity of the subject improves. In some embodiments, improvement in the visual acuity of the subject is assessed by an improvement in a ETDRS letter chart assessment. In some embodiments, an improvement in the ETDRS letter chart assessment comprises the correct identification of at least one (e.g., at least 2, at least 3, at least 4, at least 5, at least 7, at least 10, at least 12, 1, 2, 3, 4, 5, 6, 7, 8, 9, 10, 11, or 12) additional letter in comparison to the number of letters correctly identified before administration of the dual N-type and L-type calcium channel blocker selective for the N-type calcium channel to the subject. In some embodiments, the subject is identified or diagnosed as having an abnormally high intraocular pressure. In some embodiments, the subject is identified or diagnosed as having glaucoma.

In some embodiments, after administration of the dual N-type and L-type calcium channel blocker selective for the N-type calcium channel to the subject, an ocular symptom of the subject improves. In some embodiments, the improvement is assessed by a Physician's Assessment of Disease. In some embodiments, the improvement is assessed by an improvement in a 1-10 scale. In some embodiments, the ocular symptoms comprise eye pain, dry eye disease, a symptom associated with an eyelid, a conjunctival symptom, or any combination thereof.

In some embodiments, after administering the dual N-type and L-type calcium channel blocker selective for the N-type calcium channel to the subject, a decrease in tear fluid osmolarity occurs in an eye of the subject.

In some embodiments, after administering the dual N-type and L-type calcium channel blocker selective for the N-type calcium channel to the subject, an increase in ocular surface moistness occurs in an eye of the subject. In some embodiments, the increase in ocular surface moistness comprises an improvement of meibomian gland dysfunction. In some embodiments, an improvement of meibomian gland dysfunction occurs through improvement of autonomic functioning.

In some embodiments, after administering the dual N-type and L-type calcium channel blocker selective for the N-type calcium channel to the subject, a reduction in thermal hyperalgesia, cool hyperalgesia, or both occurs in an eye of the subject.

In some embodiments, after administering the dual N-type and L-type calcium channel blocker selective for the N-type calcium channel to the subject, an increase in blood flow occurs in an eye of the subject. In some embodiments, after administering the the dual N-type and L-type calcium channel blocker selective for the N-type calcium channel to the subject, vasodilation occurs in an eye of the subject.

In some embodiments, after administering the dual N-type and L-type calcium channel blocker selective for the N-type calcium channel to the subject, an improvement in the functioning and/or the population of amacrine cells occurs in the subject.

In some embodiments, the subject is a female. In some embodiments, the subject is a male. In some embodiments, the age of the subject is from 20 to 60 (e.g., from 25 to 55, from 25 to 50, from 25 to 45, from 25 to 40, from 25 to 35, from 35 to 60, from 35 to 55, from 35 to 50, from 35 to 45, from 37 to 43, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60).

In some embodiments, the treating comprises alleviating one or more symptoms associated with eye pain, an eye disease, or an eye disorder in the subject. In this context, alleviating one or more symptoms associated with eye pain, an eye disease, or an eye disorder can, for example, comprise reducing the severity, duration, and/or frequency of the symptoms when compared to (1) the severity, duration, and/or frequency of the one or more symptoms in the subject before start of the treatment (e.g., before administration of the dual N-type and L-type calcium channel blocker selective for the N-type calcium channel, and wherein the severity, duration, and/or frequency of the one or more symptoms before administration of the dual N-type and L-type calcium channel blocker selective for the N-type calcium channel can, for example, be evaluated by a single measurement or assessment, or an average of a plurality of measurements or assessments taken, e.g., over the course of a 2 week period, a 7 day period, a 6 day period, a 5 day period, a 4 day period, a 3 day period, a 2 day period, or a 1 day period (e.g., a 7 day period)), wherein, for example, the reduction in severity, duration, and/or frequency of the symptoms is measured about 1 hour after treatment (e.g., after about 2 hours, 4 hours, 6 hours, 8 hours, 16 hours, 1 day, 2 days, 3 days, 4 days, 5 days, 6 days, 1 week, 1.5 weeks, 2 weeks, 3 weeks, 4 weeks, 6 weeks, 2 months, 3 months, or 1 year of treatment); (2) the severity, duration, and/or frequency of the one or more symptoms experienced by a subject after the subject was administered a placebo; and/or (3) the severity, duration, and/or frequency of the one or more symptoms experienced by a subject after the subject was administered an alternative treatment such as a non-N selective calcium channel blocker. In some embodiments, the reduction in severity, duration, and/or frequency of the symptoms is greatest within 2 days (e.g., within 1.5 days, within 1 day, within 20 hours, within 16 hours, within 12 hours, within 8 hours, within 6 hours, within 4 hours, within 2 hours, or within 1 hour after administration of the dual N-type and L-type calcium channel blocker selective for the N-type calcium channel. In some embodiments, the reduction in severity, duration, and/or frequency of the symptoms is greatest within 8 hours after administration of the dual N-type and L-type calcium channel blocker selective for the N-type calcium channel.

In some embodiments, after administration of the dual N-type and L-type calcium channel blocker selective for the N-type calcium channel in the subject, intracellular calcium influx in tissue and/or macrophages is reduced. It is understood that the reduction in intracellular calcium levels is a direct result of blockade of calcium channels and furthermore can be attributed to inhibition of the TRPV-1 peripheral nociceptor ending.

In some embodiments, the non-N-selective calcium channel blocker is a dihydropyridine. In some other embodiments, the non-N-selective calcium channel blocker is a non-dihydropyridine. Non-limiting examples of non-N-selective calcium channel blockers include, but are not limited to: nifedipine, nicardipine, amlodipine, Z-944, nimodipine, verapamil, diltiazem, felodipine, isradipine, nisoldipine, mibafredil, nilvidipine barnidipine, benidipine lacidipine, lercanidipine, manidipine and nitrendipine, and pharmaceutically acceptable salts thereof. In some embodiments, the non-N-selective calcium channel blocker is amlodipine, nifedipine, nicardipine, nimodipine, diltiazem or verapamil.

In some embodiments, after administration of the dual N-type and L-type calcium channel blocker selective for the N-type calcium channel autonomic function of the subject is improved; wherein the improvement of autonomic functioning in the subject is characterized by a lower reduction in systolic blood pressure in the upper arm (e.g., the portion of the arm between the elbow and shoulder, inclusive of the elbow and shoulder) of the subject when the subject is subjected to a tilt table test. For information on the tilt table test, see, for example, Clin. Auton. Res. 2019, 29(2), 215-230, which is incorporated herein in its entirety.

In some embodiments, after administration of the dual N-type and L-type calcium channel blocker selective for the N-type calcium channel in the subject, one or both of sympathetic tone diminution and direct smooth muscle relaxation, occur in the subject.

In some embodiments, vasoconstriction in the subject is reduced after administering the dual N-type and L-type calcium channel blocker selective for the N-type calcium channel to the subject. In some embodiments, the subject is identified or diagnosed as having vasoconstriction, and the vasoconstriction in the subject is reduced after administering the dual N-type and L-type calcium channel blocker selective for the N-type calcium channel to the subject. In some embodiments, the vasoconstriction comprises vasoconstriction of a body part. In some embodiments, the temperature of the vasoconstricted body part is lower than the subject's body temperature.

In some embodiments, the subject has or is being treated for Raynaud's syndrome, lupus, scleroderma, scleroderma with interstitial lung disease, rheumatoid arthritis, atherosclerosis, cryoglobulinemia, polycythemia, dermatomyositis, polymyositis, Sjögren's syndrome, or any combination thereof. In some embodiments, the treatment for lupus, scleroderma, scleroderma with interstitial lung disease, rheumatoid arthritis, atherosclerosis, cryoglobulinemia, polycythemia, dermatomyositis, polymyositis, Sjögren's syndrome, or any combination thereof comprises administering a therapeutic agent. Therapeutic agents known in the art for treating lupus, scleroderma, scleroderma with interstitial lung disease, rheumatoid arthritis, atherosclerosis, cryoglobulinemia, polycythemia, dermatomyositis, polymyositis, and Sjögren's syndrome can be found in, e.g., the Physicians' desk reference. (71st ed.). (2017). Montvale, NJ: PDR Network.

In some embodiments, the subject has scleroderma. In some embodiments, the scleroderma is limited scleroderma. In some embodiments, the scleroderma is diffuse scleroderma. In some embodiments, after administering the dual N-type and L-type calcium channel blocker selective for the N-type calcium channel one or more symptoms of the scleroderma are improved. In some embodiments, the one or more symptoms of the scleroderma are selected from hardening and tightening of skin, shiny skin, restricted movement of the affected part due to hardness in skin, hair loss, white lumps under the skin (due to, e.g., calcium deposition), exaggerated responses to cold temperatures and emotional stress, numbness in a finger or toe, pain in a finger or toe, changes in skin color of a finger or toe, acid reflux, restricted movement of food through the digestive tract, and malnutrition.

In some embodiments, the subject has (e.g., is identified or diagnosed as having) Raynaud's syndrome. In some embodiments, the Raynaud's syndrome is selected from the group consisting of: primary Raynaud's syndrome; secondary Raynaud's syndrome; Raynaud's syndrome of the nipple, nose, ear, penis, tongue, and/or any alar circulatory region. In some embodiments, the Raynaud's syndrome is primary Raynaud's syndrome. In some embodiments, the Raynaud's syndrome is secondary Raynaud's syndrome.

In some embodiments, after administration of the dual N-type and L-type calcium channel blocker selective for the N-type calcium channel one or more symptoms of the Raynaud's syndrome are improved.

In some embodiments, the symptoms are selected from the group consisting of: pain, anemia, fatigue, change in coloration of the skin, cyanosis, reperfusion, deoxygenation of the blood, digital ulcerations, reduced temperature in one or more parts of the body, changes in the endothelium of a blood vessel, swelling, impaired vision, or any combination thereof. In some embodiments, the symptom is pain.

In some embodiments, after administration of the dual N-type and L-type calcium channel blocker selective for the N-type calcium channel to the subject, the cardiac function of the subject is improved. In some embodiments, improving the cardiac function in the subject comprises improving the left ventricular function of the subject. In some embodiments, the subject has hypertension. In some embodiments, the subject does not have hypertension.

In some embodiments, the subject has hypertension; and after administration of the dual N-type and L-type calcium channel blocker selective for the N-type calcium channel to the subject, the blood pressure of the subject is decreased and the cardiac function (e.g., left ventricular function) of the subject is improved.

In some embodiments, the subject does not have hypertension; and after administration of the dual N-type and L-type calcium channel blocker selective for the N-type calcium channel to the subject, the blood pressure of the subject is not decreased and the cardiac function (e.g., left ventricular function) of the subject is improved.

In some embodiments, the subject has hypertension and osteoporosis; and after administration of the dual N-type and L-type calcium channel blocker selective for the N-type calcium channel to the subject, the blood pressure of the subject is decreased and the bone density in the subject is increased.

In some embodiments, the subject does not have hypertension; the subject has osteoporosis; and after administration of the dual N-type and L-type calcium channel blocker selective for the N-type calcium channel to the subject, the blood pressure of the subject is not decreased and the bone density in the subject is increased.

In some embodiments, the subject has hypertension; and after administration of the dual N-type and L-type calcium channel blocker selective for the N-type calcium channel to the subject, the blood pressure of the subject is decreased and the bone density in the subject is increased.

In some embodiments, the subject does not have hypertension; and after administration of the dual N-type and L-type calcium channel blocker selective for the N-type calcium channel to the subject, the blood pressure of the subject is not decreased and the bone density in the subject is increased.

In some embodiments, the subject has hypertension and atherosclerosis; and after administration of the dual N-type and L-type calcium channel blocker selective for the N-type calcium channel to the subject, the blood pressure of the subject is decreased and the atherosclerosis in the subject is improved. In some embodiments, the subject exhibits a reduced amount of plaque deposition in a carotid artery. In some embodiments, the reduced plaque deposition is measured by ultrasound or magnetic resonance imaging.

In some embodiments, the subject does not have hypertension; the subject has atherosclerosis; and after administration of the dual N-type and L-type calcium channel blocker selective for the N-type calcium channel to the subject, the blood pressure of the subject is not decreased and the atherosclerosis in the subject is improved. In some embodiments, the subject exhibits a reduced amount of plaque deposition in a carotid artery. In some embodiments, the reduced plaque deposition is measured by ultrasound or magnetic resonance imaging.

In some embodiments, the subject has hypertension; and after administration of the dual N-type and L-type calcium channel blocker selective for the N-type calcium channel to the subject, the blood pressure of the subject is decreased and renal function in the subject is improved.

In some embodiments, the subject does not have hypertension; and after administration of the dual N-type and L-type calcium channel blocker selective for the N-type calcium channel to the subject, the blood pressure of the subject is not decreased and renal function in the subject is improved.

In some embodiments, the subject has hypertension; and after administration of the dual N-type and L-type calcium channel blocker selective for the N-type calcium channel to the subject, the blood pressure of the subject is decreased and renal function in the subject is improved. In some embodiments, the subject does not have hypertension; and after administration of the dual N-type and L-type calcium channel blocker selective for the N-type calcium channel to the subject, the blood pressure of the subject is not decreased and renal function in the subject is improved.

In some embodiments, the subject has hypertension; the subject was previously treated with antihypertensive agents before administration of the dual N-type and L-type calcium channel blocker selective for the N-type calcium channel; and after administration of the dual N-type and L-type calcium channel blocker selective for the N-type calcium channel to the subject, the blood pressure of the subject is decreased.

In some embodiments, the subject does not have hypertension; the subject was previously treated with antihypertensive agents before administration of the dual N-type and L-type calcium channel blocker selective for the N-type calcium channel; and after administration of the dual N-type and L-type calcium channel blocker selective for the N-type calcium channel to the subject, the blood pressure of the subject is is not decreased.

In some embodiments, the subject has hypertension; the subject has scleroderma; the subject has a digital ulcer; and after administration of the dual N-type and L-type calcium channel blocker selective for the N-type calcium channel to the subject, the blood pressure of the subject is decreased, and the digital ulcer is treated. In some embodiments, treating the digital ulcer comprises healing or improving the condition of the digital ulcer.

In some embodiments, the subject does not have hypertension; the subject has scleroderma; the subject has a digital ulcer; and after administration of the dual N-type and L-type calcium channel blocker selective for the N-type calcium channel to the subject, the blood pressure of the subject is not reduced, and the digital ulcer is treated. In some embodiments, treating the digital ulcer comprises healing or improving the condition of the digital ulcer.

In some embodiments, after administration of the dual N-type and L-type calcium channel blocker selective for the N-type calcium channel in the subject, norepinephrine and metabolites thereof (e.g., norepinephrine) are reduced in the subject. In some embodiments, after administration of the dual N-type and L-type calcium channel blocker selective for the N-type calcium channel in the subject, norepinephrine is reduced in the subject. In some embodiments, after administration of the dual N-type and L-type calcium channel blocker selective for the N-type calcium channel in the subject, circulating plasma concentration of norepinephrine and metabolites thereof are reduced in the subject.

In some embodiments, the dual N-type and L-type calcium channel blocker selective for the N-type calcium channel exhibits a greater selectivity for the N-type calcium channel over the L-type calcium channel than a non-N-selective calcium channel blocker.

In some embodiments, the therapeutically effective amount of the dual N-type and L-type calcium channel blocker selective for the N-type calcium channel is at least 10% lower than the therapeutically effective amount of the non-N-selective calcium channel blocker. For example, the therapeutically effective amount of the dual N-type and L-type calcium channel blocker selective for the N-type calcium channel is at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 90% lower, or at least 95% lower than the therapeutically effective amount of the non-N-selective calcium channel blocker.

In some embodiments, the therapeutically effective amount of the dual N-type and L-type calcium channel blocker selective for the N-type calcium channel decreases the blood pressure (e.g., the systolic blood pressure) of the subject to a lesser degree than the therapeutically effective amount of the non-N-selective calcium channel blocker. In some embodiments, the dual N-type and L-type calcium channel blocker selective for the N-type calcium channel decreases the blood pressure (e.g., the systolic blood pressure) of the subject at least 5% less than the non-N-selective calcium channel blocker. For example, the dual N-type and L-type calcium channel blocker selective for the N-type calcium channel decreases the blood pressure (e.g., the systolic blood pressure) of the subject at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, or at least 95% less, than the non-N-selective calcium channel blocker.

In some embodiments, following the administration of the therapeutically effective amount of the dual N-type and L-type calcium channel blocker selective for the N-type calcium channel, the subject experiences reduced hypotension, lower extremity edema, and/or headache than a subject administered a therapeutically effective amount of a non-N selective calcium channel blocker. In some embodiments, the hypotension is arterial hypotension.

In some embodiments, the dual N-type and L-type calcium channel blocker selective for the N-type calcium channel is more effective than the non-N-selective calcium channel blocker in treating an adverse effect of the eye disease or disorder. In some embodiments, the adverse effect is a symptom or a clinical manifestation of the complex regional pain syndrome. In some embodiments, the subject experiences less frequent, less severe, and/or shorter episodes of symptoms and/or adverse events than when administered a therapeutically effective amount of a non-N-selective calcium channel blocker useful to treat the complex regional pain syndrome. In some embodiments, the adverse events are one or more events selected from the group consisting of: pain (e.g., neuropathic pain), dry eye, vision loss (e.g., loss of peripheral vision), seeing halos around lights, seeing glare in bright light, eye redness, haziness in eye, tunnel vision, burning pain in eye, foreign body sensation in eye, photophobia, swelling in an eye, sensitivity to light, floaters in field of vision, night blindness, blurred vision, alteration in color of iris, abnormally small pupil size, or excessive tearing. In some embodiments, the subject experiences less frequent, less severe, and/or shorter episodes of pain (e.g., neuropathic pain) than when administered a therapeutically effective amount of a non-N-selective calcium channel blocker useful to treat the complex regional pain syndrome.

In some embodiments, the therapeutically effective amount of the dual N-type and L-type calcium channel blocker selective for the N-type calcium channel is reduced compared to the therapeutically effective amount of a non-N-selective calcium channel blocker useful to treat the complex regional pain syndrome.

In some embodiments, one or more side effects experienced by the subject after administration of the dual N-type and L-type calcium channel blocker selective for the N-type calcium channel are less severe or less frequent than as compared to the side effects experienced by a subject after administration of a therapeutically effective amount of a non-N-selective calcium channel blocker useful to treat the disease or disorder. Without wishing to be bound by theory, this may allow a higher dose of the dual N-type and L-type calcium channel blocker selective for the N-type calcium channel to be administered to the subject, which can, e.g., result in a higher treatment efficacy than the non-N-selective calcium channel blocker. In some embodiments, the side effects are selected from dizziness, peripheral edema, lower extremity edema, flushing, flushing sensation, acute myocardial infarction, muscle cramps, tremor, cough, dyspnea, hypotension, wheezing, and increased gastroesophageal reflux.

It has been shown that dual N-type and L-type calcium channel blockers selective for the N-type calcium channels have fewer and less severe side effects, better tolerability, and are safer than non-N-selective calcium channel blockers. It is believed that this is due to the increased inhibition of the N channel relative to the L channel. By decreasing sympathetic activity, as well as by dilating not only arterioles but the venous system, dual N-type and L-type calcium channel blockers selective for the N-type calcium channel appear to be associated with less adverse events in patients treated for hypertension than patients treated with dual L and N-calcium channel antagonists with lower levels of N-selectivity.

It is understood that dual N-type and L-type calcium channel blocker selective for the N-type calcium channel may decrease the blood pressure of subjects that are hypertensive. As such, it may be beneficial to administer an agent that increases blood pressure in combination with the dual N-type and L-type calcium channel blocker selective for the N-type calcium channel. In some embodiments of the methods disclosed herein, the method further comprises administering to the subject a therapeutically effective amount of an agent that increases blood pressure. In some embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines. In some embodiments, the blood pressure of the subject before and after administration of the dual N-type and L-type selective calcium blocker and the agent that increases blood pressure is substantially the same. In some other embodiments, the blood pressure of the subject after administration of the dual N-type and L-type selective calcium blocker and the agent that increases blood pressure is less than 20% (e.g., less than 15%, less than 10%, less than 5%, less than 3% or less than 1%) higher or lower than the blood pressure of the subject before administration of the dual N-type and L-type selective calcium blocker and the agent that increases blood pressure.

In some embodiments, the treating comprises reducing pulmonary hypertension in the subject.

In some embodiments, the subject is also diagnosed with hypertension; and wherein after administration of the dual N-type and L-type calcium channel blocker selective for the N-type calcium channel to the subject, the blood pressure (e.g., systolic blood pressure) of the subject is reduced. In some embodiments, the subject was not diagnosed with hypertension; and wherein after administration of the dual N-type and L-type calcium channel blocker selective for the N-type calcium channel to the subject, the blood pressure (e.g., the systolic blood pressure) of the subject is not reduced. Without wishing to be bound by theory, it is believed that when the subject has hypertension, the dual N-type and L-type calcium channel blocker selective for the N-type calcium channel reduces the blood pressure of the subject; however, when the subject does not have hypertension (i.e., the subject is normotensive), the dual N-type and L-type calcium channel blocker selective for the N-type calcium channel does not reduce the blood pressure of the subject.

In some embodiments, the systolic blood pressure of the subject is reduced by greater than about 1 mm Hg (e.g., greater than about 2 mm Hg, greater than about 5 mm Hg, greater than about 10 mm Hg, greater than about 15 mm Hg, greater than about 20 mm Hg, greater than about 30 mm Hg, or greater than about 40 mm Hg). In some embodiments, the systolic blood pressure of the subject is reduced by greater than 10 mm Hg.

In some embodiments, the subject is identified as normotensive; and wherein after administration of the dual N-type and L-type calcium channel blocker selective for the N-type calcium channel to the subject, the systolic blood pressure of the subject changes by less than 20% (e.g., less than 18%, less than 16%, less than 14%, less than 12%, less than 10%, less than 8%, less than 6%, less than 5%, less than 4%, less than 3%, less than 2%, or less than 1%). In some embodiments, the subject is identified as normotensive; and wherein after administration of the dual N-type and L-type calcium channel blocker selective for the N-type calcium channel to the subject, the systolic blood pressure of the subject changes by less than 5%. In some embodiments, the subject is identified as normotensive; and wherein after administration of the dual N-type and L-type calcium channel blocker selective for the N-type calcium channel to the subject, the systolic blood pressure of the subject remains substantially the same. In some embodiments, the subject is identified as normotensive; and wherein after administration of the dual N-type and L-type calcium channel blocker selective for the N-type calcium channel to the subject, the heart rate and systolic blood pressure of the subject remains substantially the same.

In some embodiments, after administration of the dual N-type and L-type calcium channel blocker selective for the N-type calcium channel, the bone density of the subject does not decrease. In some of these embodiments, the bone density of the subject increases. This may occur through a reduction in the number of osteoclasts in the subject and/or an increase in the ratio of alkaline phosphate to tartrate resistant acid phosphatase (TRAP).

In some embodiments, the method further comprises selecting a subject identified or diagnosed as having reduced bone density for the treatment. In some embodiments, the subject identified or diagnosed as having reduced bone density has osteoporosis. In some embodiments, the subject is female.

In some embodiments, the method further comprises selecting a subject identified or diagnosed as having reduced renal function for the treatment. In some embodiments, the renal function of the subject is not reduced after treatment. In some embodiments, the renal function of the subject is improved after treatment.

In some embodiments, improving renal function comprises determining one or more of: a reduction in intrarenal arterial stiffness, improved blood flow to the kidneys, increased expression levels of podocyte proteins, reduction in urinary protein excretion, improvement in glomerular filtration rate, reduction in plasma creatinine, decrease in brachial-ankle pulse wave velocity, improvement in plasma inulin clearance, or any combination thereof. In some embodiments, improved renal function comprises a reduction in intrarenal arterial stiffness, improved blood flow to the kidneys, increased expression levels of podocyte proteins, or any combination thereof.

In some embodiments, after administration of the dual N-type and L-type calcium channel blocker selective for the N-type calcium channel, sympathetic tone diminution, direct smooth muscle relaxation, dysesthetic pain, burning pain, body temperature changes of the subject, hyperesthesia, changes in skin or tissue color, edema, changes in skin turgor, ruble, pallor, cyanosis, vasospasm, or any combination thereof are treated in the subject. In this context, “treated” can, for example, refer to ameliorated or improved. In some embodiments, after administration of the dual N-type and L-type calcium channel blocker selective for the N-type calcium channel, sympathetic tone diminution, direct smooth muscle relaxation, dysesthetic pain, burning pain, body temperature changes of the subject, changes in skin or tissue color, edema, changes in skin turgor, ruble, pallor, cyanosis, vasospasm, or any combination thereof are treated in the subject. In some embodiments, after administration of the dual N-type and L-type calcium channel blocker selective for the N-type calcium channel, a reduction in sympathetic tone diminution, direct smooth muscle relaxation, dysesthetic pain, burning pain, body temperature changes of the subject, hyperesthesia, changes in skin or tissue color, edema, changes in skin turgor, ruble, pallor, cyanosis, vasospasm, or any combination thereof is observed in the subject. In some embodiments, after administration of the dual N-type and L-type calcium channel blocker selective for the N-type calcium channel, a reduction in sympathetic tone diminution, direct smooth muscle relaxation, dysesthetic pain, burning pain, body temperature changes of the subject, changes in skin or tissue color, edema, changes in skin turgor, ruble, pallor, cyanosis, vasospasm, or any combination thereof are treated in the subject.

In some embodiments, after administration of the dual N-type and L-type calcium channel blocker selective for the N-type calcium channel to the subject, nitric oxide is increased in the subject.

In some embodiments, after administration of the dual N-type and L-type calcium channel blocker selective for the N-type calcium channel to the subject, nitric oxide is not increased in the subject.

In some embodiments, after administration of the dual N-type and L-type calcium channel blocker selective for the N-type calcium channel endothelial dysfunction in the subject is improved.

In some embodiments, after administration of the dual N-type and L-type calcium channel blocker selective for the N-type calcium channel oxidative stress in the subject is decreased. In some embodiments, decreasing oxidative stress in the subject comprises decreasing oxidative stress in the subject after administration of the dual N-type and L-type calcium channel blocker selective for the N-type calcium channel relative to the oxidative stress in the subject before administration of the dual N-type and L-type selective calcium blocker.

In some embodiments, an antioxidant selected from the group consisting of a hydralazine compound, a glutathione, vitamin C, cysteine, I3-carotene, a ubiquinone, a ubiquinol-10, a tocopherol, coenzyme Q, or a mixture thereof is not administered to the subject. In some embodiments, an anti-oxidant is not administered to the subject.

In some embodiments, the subject is a mammal. In some embodiments, the subject is human. In some embodiments, the subject is female. In some embodiments, the subject is male.

In some embodiments, the dual N-type and L-type calcium channel blocker selective for the N-type calcium channel exhibits at least a 2-fold selectivity (e.g., at least a 4-fold, 10-fold, 20-fold, 30-fold, 40-fold, 50-fold, 60-fold, 80-fold, 100-fold, 130-fold, 150-fold, or 200-fold selectivity) for the N-type calcium channel over an L-type calcium channel. In some embodiments, the dual N-type and L-type calcium channel blocker selective for the N-type calcium channel exhibits at least a 50-fold selectivity for the N-type calcium channel over an L-type calcium channel. In some embodiments, the dual N-type and L-type calcium channel blocker selective for the N-type calcium channel exhibits a 50-fold to 100-fold selectivity for the N-type calcium channel over an L-type calcium channel.

In some embodiments, the dual N-type and L-type calcium channel blocker selective for the N-type calcium channel exhibits a greater selectivity for the N-type calcium channel over the L-type calcium channel than a non-N-selective calcium channel blocker. In some embodiments, the dual N-type and L-type calcium channel blocker selective for the N-type calcium channel exhibits at least a 5% (e.g., at least a 10%, 20%, 30%, 40%, 50%, 60%, 70%, 80%, 100%, 150%, 200%, 300%) greater selectivity for the N-type calcium channel over the L-type calcium channel than a non-N-selective calcium channel blocker.

In some embodiments, the dual N-type and L-type calcium channel blocker selective for the N-type calcium channel is selected from the group consisting of:

cilnidipine, Z-160, CNV2197944, or pharmaceutically acceptable salts thereof. In some embodiments, the dual N-type and L-type calcium channel blocker selective for the N-type calcium channel is cilnidipine or a pharmaceutically acceptable salt thereof.

In some embodiments, the method comprises administering at least one additional therapeutic agent to the subject. The at least one additional therapeutic agent can be administered simultaneously, separately, sequentially, or in combination (e.g., for more than two agents) with the dual N-type and L-type calcium channel blocker selective for the N-type calcium channel. Non-limiting examples of additional therapeutic agents include calcium channel blockers, sodium channel blockers (e.g., Nav 1.7 sodium channel blocker), TRP-v1 inhibitors, and therapeutic agents that relieve pain.

In some embodiments, the at least one additional therapeutic agent is a phosphodiesterase type 5 inhibitor. In some embodiments, the phosphodiesterase type 5 inhibitor is sildenafil or tadalafil. In some embodiments, the phosphodiesterase type 5 inhibitor is tadalafil. In some embodiments, the method comprises administering about 2 mg to about 50 mg (e.g., about 2 mg to about 30 mg, about 2 mg to about 20 mg, about 5 mg to about 10 mg, about 2 mg, about 5 mg, about 10 mg, about 15 mg, about 20 mg, about 25 mg, about 30 mg, about 35 mg, about 40 mg, about 45 mg, or about 50 mg) tadalafil to the subject. In some embodiments, the method comprises administering about 5 mg tadalafil to the subject.

In some embodiments, the at least one (e.g., one) additional therapeutic agent is used to treat nociceptive mechanical pain. In some embodiments, the at least one (e.g., one) additional therapeutic agent is used to treat ocular neuropathic pain.

In some embodiments, the at least one (e.g., one) additional therapeutic agent is selected from the group consisting of: a beta blocker, an alpha agonist, a carbonic anhydrase inhibitor, a cholinergic, a prostaglandin, a prostamide, and combinations thereof. In some embodiments, the beta blocker is brimonidine or timolol. In some embodiments, the beta blocker is timolol. In some embodiments, the carbonic anhydrase inhibitor is Diamox or dorzolamide. In some embodiments, the cholinergic is pilocarpine. In some embodiments, the prostaglandin is latanoprost. In some embodiments, the prostamide is bimatoprost. In some embodiments, the at least one additional therapeutic agent is selected from the group consisting of: brimonidine, timolol, Diamox, dorzolamide, pilocarpine, latanoprost, and bimatoprost. In some embodiments, the at least one additional therapeutic agent is a beta blocker. In some embodiments, the at least one additional therapeutic agent is timolol.

In some embodiments, the at least one additional therapeutic agent facilitates aqueous humor drainage from the eye through the trabecular network. In some embodiments, the at least one additional therapeutic agent is selected from the group consisting of: Prostaglandins e.g., Xalatan (latanoprost), Travatan Z (travoprost), Zioptan (tafluprost), and Lumigan (bimatoprost)); Rho kinase inhibitors, (e.g., Rhopressa (netarsudil); Nitric oxides (e.g., Vyzulta (latanoprostene bunod)); Miotic or cholinergic agents (e.g., Isopto Carpine (pilocarpine)); and combinations thereof. In some embodiments, the at least one additional therapeutic agent reduces the rate at which fluid (e.g., tears) is generated by the eye. In some embodiments, the at least one additional therapeutic agent is selected from the group consisting of: Alpha-adrenergic agonists (e.g., Iopidine (apraclonidine) and Alphagan P or Qoliana (brimonidine); Beta blockers (e.g., Betoptic (betaxolol) and Betimol, Istalol, or Timoptic (timolol)); Carbonic anhydrase inhibitors (e.g., Trusopt (dorzolamide) and Azopt (brinzolamide)); and any combination thereof.

In some embodiments, the at least one additional therapeutic agent comprises an analgesic. In some embodiments, the analgesic comprises fentanyl or bupivacaine. In some embodiments, the at least one additional therapeutic agent comprises an anti-inflammatory agent. In some embodiments, the anti-inflammatory agent is a steroid (e.g., fluorometholone or loteprednol) or a non-steroidal anti-inflammatory drug (NSAID) (e.g., cyclosporine or lifitegrast). Without wishing to be bound by theory, it is believed that short courses of topical steroids have been shown to be useful to decrease surface inflammation, and, in some embodiments, stops a cycle of inflammation and nerve damage and provides symptomatic relief. Generally these agents are tapered and discontinued once the inflammation is controlled to prevent formation of cataracts and increased intraocular pressure. Non-steroidal anti-inflammatory therapies are understood to decrease surface inflammation and modulate T cell mediated inflammation.

In some embodiments, the at least one additional therapeutic agent comprises a tri-cyclic antidepressant (e.g., amytriptyline, nortriptyline), an anti-convulsants (e.g. carbamazepine), dronabinol, tramadol, gabapentin, pregabalin, or any combination thereof.

In some embodiments, the at least one additional therapeutic agent comprises Vitamin B (e.g., vitamin B12) or Vitamin D.

In some embodiments, the at least one additional therapeutic agent comprises Botulinum toxin A.

In some embodiments, the subject is treated with or subjected to at least one additional therapy. In some embodiments, the at least one additional therapy comprises an ocular surface treatment. In some embodiments, the ocular surface treatment comprises applying eye lubrication, artificial tears, fish oil, flax seed oil, or a punctal plug to the eye of the subject. Without wishing to be bound by theory, eye lubrication or artificial tears are believed to decrease the hyperosmolarity of tears and reduce overstimulation of the nociceptors. In some embodiments (for example, when the subject is diagnosed with blepharitis), topical and/or systemic antibiotics are administered to the subject. In some embodiments, scleral lenses, (e.g., prosthetic replacement of the ocular surface ecosystem (PROSE)) are applied to the eye of the subject. Without wishing to be bound by theory, it is believed that PROSE ameliorates post LASIK corneal neuralgia.

In some embodiments, the at least one additional therapy comprises a neuroregenerative therapy. Without wishing to be bound by theory, neuroregenerative therapies are believed to target the underlying pathophysiology of aberrant nerve regeneration subsequent to repeated injury in neuropathic pain. In some embodiments, the neuroregenerative therapy comprises contacting the eye of the subject with serum tears.

In some embodiments, the at least one additional therapy comprises electrical neurostimulation or trans-magnetic stimulation. In some embodiments, electrical neurostimulation comprises direct neuro-stimulation of the large diameter afferent fibers along the corneal pain pathway using percutaneously placed monopolar electrodes.

In some aspects, disclosed herein is a method of treating eye pain in a subject in need thereof, the method comprising administering to the subject (a) a therapeutically effective amount of a dual N-type and L-type calcium channel blocker selective for the N-type calcium channel, and (b) at least one additional therapeutic agent.

In some embodiments, the dual N-type and L-type calcium channel blocker selective for the N-type calcium channel does not include a tetravalent (i.e., quaternized) nitrogen wherein the four substituents bonded to the nitrogen are non-hydrogen substituents.

In some embodiments, the dual N-type and L-type calcium channel blocker selective for the N-type calcium channel does not include a sulfide (i.e., —S—) moiety.

In some embodiments of any method disclosed herein, the subject is not infected with SARS-CoV-2. In some embodiments of any method disclosed herein, the subject is not afflicte with (i.e., not suffering from) SARS-CoV-2 infection. In some embodiments of any method disclosed herein, the subject is not being treated for SARS-CoV-2. In some embodiments of any method disclosed herein, the subject is not diagnosed with SARS-CoV-2 (e.g., after the subject is subjected to a test that diagnoses whether the subject is infected with SARS-CoV-2 (e.g., a PCR test, an antigen test, or an antibody test)).

In some embodiments of any method disclosed herein, the subject is infected with SARS-CoV-2. In some embodiments of any method disclosed herein, the subject is afflicted with (i.e., suffering from) SARS-CoV-2. In some embodiments of any method disclosed herein, the subject is being treated for SARS-CoV-2 infection. In some embodiments of any method disclosed herein, the subject is diagnosed with SARS-CoV-2 infection (e.g., after the subject is subjected to a test that diagnoses whether the subject is infected with SARS-CoV-2 (e.g., a PCR test, an antigen test, or an antibody test)).

In some embodiments, greater than about 2% (e.g., greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, or greater than about 65%, greater than about 70%, greater than about 75%, greater than about 80%, greater than about 85%, greater than about 90%, greater than about 95%, greater than about 97%, or greater than about 99%) of the dual N-type and L-type calcium channel blocker selective for the N-type calcium channel is anionic at physiological pH (e.g., a pH of from from about 7.2 to about 7.6 (e.g., about 7.4)).

In some embodiments, the method further comprises determining that the subject has sympathetic overactivity or elevated sympathetic outflow (e.g., before administration of the dual N-type and L-type calcium channel blocker selective for the N-type calcium channel to the subject). In some embodiments, the method further comprises identifying a subject having sympathetic overactivity or elevated sympathetic outflow (e.g., before administration of the dual N-type and L-type calcium channel blocker selective for the N-type calcium channel to the subject). In some embodiments, the method further comprises determining whether the subject has a clinical record indicating sympathetic overactivity or elevated sympathetic outflow (e.g., before administration of the dual N-type and L-type calcium channel blocker selective for the N-type calcium channel to the subject). In some embodiments, the method further comprises monitoring sympathetic activity or sympathetic outflow in the subject during treatment. In some embodiments, the method further comprises determining a decrease in sympathetic activity or sympathetic outflow in the subject after treatment. In some embodiments, determining, identifying, or monitoring sympathetic activity or sympathetic outflow in the subject is performed using galvanic skin testing and/or plethysmography

In some embodiments, the dual N-type and L-type calcium channel blocker selective for the N-type calcium channel further inhibits a Nav 1.7 sodium channel.

In some embodiments, following the administration of the therapeutically effective amount of the dual N-type and L-type calcium channel blocker selective for the N-type calcium channel, the subject has an increase in Nav 1.7 inhibition. In some embodiments, the Nav 1.7 inhibition comprises inhibition of the closed state of the Nav 1.7 sodium channel. In some embodiments, the Nav 1.7 inhibition comprises inhibition of the inactivated state of the Nav 1.7 sodium channel. In some embodiments, the inhibition of the closed state of the Nav 1.7 sodium channel is greater than the inhibition of the inactivated state of the Nav 1.7 sodium channel. In some embodiments, the inhibition of the inactivated state of the Nav 1.7 sodium channel is greater than inhibition of the closed state of the Nav 1.7 sodium channel.

In some embodiments, following the administration of the therapeutically effective amount of the dual N-type and L-type calcium channel blocker selective for the N-type calcium channel, the subject has an at least 1% increase in Nav 1.7 inhibition. For example, the subject has an at least 5%, at least 10%, at least 20%, at least 30%, at least 40%, at least 50%, at least 60%, at least 70%, at least 80%, at least 90%, or at least 95% increase in Nav 1.7 inhibition. In some embodiments, following the administration of the therapeutically effective amount of the Nav 1.7 sodium channel blocker, the subject has an at least 10% increase in Nav 1.7 inhibition. In some embodiments, following the administration of the therapeutically effective amount of the Nav 1.7 sodium channel blocker, the subject has an at least 20% increase in Nav 1.7 inhibition. In some embodiments, following the administration of the therapeutically effective amount of the Nav 1.7 sodium channel blocker, the subject has an at least 15% increase in Nav 1.7 inhibition. In some embodiments, following the administration of the therapeutically effective amount of the Nav 1.7 sodium channel blocker, the subject has an at least 35% increase in Nav 1.7 inhibition.

In some embodiments, the dual N-type and L-type calcium channel blocker selective for the N-type calcium channel further inhibits a TRP-v1 channel.

In some embodiments, the method further comprises determining that the subject has TRP-v1 overactivation (e.g., before administration of the dual N-type and L-type calcium channel blocker selective for the N-type calcium channel to the subject). In some embodiments, the method further comprises identifying a subject having TRP-v1 overactivation (e.g., before administration of the dual N-type and L-type calcium channel blocker selective for the N-type calcium channel to the subject). In some embodiments, the method further comprises determining whether the subject has a clinical record indicating or showing TRP-v1 overactivation (e.g., before administration of the dual N-type and L-type calcium channel blocker selective for the N-type calcium channel to the subject). In some embodiments, the method further comprises monitoring TRP-v1 overactivation in the subject during treatment. In some embodiments, the method further comprises determining a decrease in TRP-v1 activation or upregulation in the subject after treatment. In some embodiments, determining that the subject has TRPv-1 overactivation comprises determining an abnormally high TRP-v1 current density and capsaicin responding rate in small-sized nociceptive dorsal root ganglion (DRG) neurons. Methods for determining TRP-v1 overactivation in a subject include methods disclosed in Nature Reviews Drug Discovery, 2022, 21, 41-59.

In some embodiments, following the administration of the therapeutically effective amount of the dual N-type and L-type calcium channel blocker selective for the N-type calcium channel, the subject has an at least 1% increase in TRP-v1 inhibition. For example, the subject has an at least 5%, at least 10%, at least 20%, at least 30%, at least 40%, at least 50%, at least 60%, at least 70%, at least 80%, at least 90%, or at least 95% increase in TRP-v1 inhibition. In some embodiments, following the administration of the therapeutically effective amount of the dual N-type and L-type calcium channel blocker selective for the N-type calcium channel, the subject has an at least 10% increase in TRP-v1 inhibition. In some embodiments, following the administration of the therapeutically effective amount of the dual N-type and L-type calcium channel blocker selective for the N-type calcium channel, the subject has an at least 20% increase in TRP-v1 inhibition. In some embodiments, following the administration of the therapeutically effective amount of the dual N-type and L-type calcium channel blocker selective for the N-type calcium channel, the subject has an at least 15% increase in TRP-v1 inhibition. In some embodiments, following the administration of the therapeutically effective amount of the dual N-type and L-type calcium channel blocker selective for the N-type calcium channel, the subject has an at least 35% increase in TRP-v1 inhibition. In some embodiments, measuring the increase in TRP-v1 inhibition comprises measuring a decrease in TRP-v1 protein expression by immunofluorescence staining. See, for example, Front Pharmacol. 2019; 10: 453, which is incorporated by reference herein in its entirety.

In some embodiments, the method further comprises determining an abnormally high inflammation in the subject before administration of the dual N-type and L-type calcium channel blocker selective for the N-type calcium channel. In some embodiments, the method further comprises determining a reduction in inflammation in the subject after administration of the dual N-type and L-type calcium channel blocker selective for the N-type calcium channel. In some embodiments, the inflammation is generalized, neurogenic, or both.

In some embodiments, the method further comprises determining a reduced concentration of NF-κB in the subject after administration of the dual N-type and L-type calcium channel blocker selective for the N-type calcium channel. In some embodiments, the concentration of NF-κB is measured in the plasma of the subject.

In some embodiments, the method further comprises determining an increase in expression of phosphatidylinositol 3-kinase, phosphorylated Akt, phosphorylated glycogen synthase kinase-3 (pGSK-3b), heat shock transcription factor (HSTF-1), or any combination thereof in the subject after administration of the dual N-type and L-type calcium channel blocker selective for the N-type calcium channel.

In some embodiments, the method further comprises determining a decrease in expression of cytosolic cytochrome c, activated caspase 3, cleaved poly(ADP-ribose) polymerase (PARP), or any combination thereof in the subject after administration of the dual N-type and L-type calcium channel blocker selective for the N-type calcium channel.

In some embodiments, the method further comprises determining a reduced concentration of catecholamine, aldosterone, brain natriuretic peptide, urine liver-type fatty acid binding protein, albumin excretion ratio, or any combination thereof in the subject after administration of the dual N-type and L-type calcium channel blocker selective for the N-type calcium channel. In some embodiments, the concentration of concentration of catecholamine, aldosterone, brain natriuretic peptide, urine liver-type fatty acid binding protein, and albumin excretion ratio are measured in the the plasma of the subject.

In some embodiment, the method further comprises determining that the subject has dysregulation of ERK1 and/or ERK2 before administration of the dual N-type and L-type calcium channel blocker selective for the N-type calcium channel. In some embodiments, the method further comprises determining inhibition or downregulation of ERK1 and/or ERK2 in the subject after administration of the dual N-type and L-type calcium channel blocker selective for the N-type calcium channel.

In some embodiments, the method further comprises determining that the subject has upregulation of or abnormally elevated concentration (e.g., abnormally elevated concentration) of TNF-α, IL-1, IL-1B. IL2, IL-6, ET-I, cGRP, bradykinin, substance P, MMP9, CXCL13, histamine, nerve growth factor, prostaglandin E2, 5-hydroxytryptamine (5-HT), ATP, nitric oxide, or any combination thereof before administration of the dual N-type and L-type calcium channel blocker selective for the N-type calcium channel. In some embodiments, the method further comprises determining a lower concentration of TNF-α, IL-1, IL-1B, IL2, IL-6, ET-I, cGRP, bradykinin, substance P, MMP9, CXCL13, histamine, nerve growth factor, prostaglandin E2, 5-hydroxytryptamine (5-HT), ATP, nitric oxide, or any combination thereof in the subject after administration of the dual N-type and L-type calcium channel blocker selective for the N-type calcium channel. In some embodiments, the concentration of TNF-α, IL-1, IL-1B. IL2, IL-6, ET-I, cGRP, bradykinin, substance P, MMP9, CXCL13, histamine, nerve growth factor, prostaglandin E2, 5-hydroxytryptamine (5-HT), ATP, nitric oxide, are measured in the cerebrospinal fluid or the plasma (e.g., the plasma) of the subject.

In some embodiments, p38 activity is reduced in the subject after administration of the dual N-type and L-type calcium channel blocker selective for the N-type calcium channel.

In some embodiments, P2Y2 receptor activity is reduced in the subject after administration of the dual N-type and L-type calcium channel blocker selective for the N-type calcium channel.

In some embodiments, Microglial P2X7 receptor activity is reduced in the subject after administration of the dual N-type and L-type calcium channel blocker selective for the N-type calcium channel.

In some embodiments, PI3K and ERK pathways are upregulated in the subject after administration of the dual N-type and L-type calcium channel blocker selective for the N-type calcium channel.

In some embodiments, cytokine-induced activation of Human Neutrophils is reduced in the subject after administration of the dual N-type and L-type calcium channel blocker selective for the N-type calcium channel.

In some embodiments, the method further comprises determining that the subject has abnormally low concentration of IL-8 mRNA, IL-4, IL-10, or any combination thereof before administration of the dual N-type and L-type calcium channel blocker selective for the N-type calcium channel. In some embodiments, the method further comprises determining that the concentration of IL-8 mRNA, IL-4, IL-10, or any combination thereof increased in the subject after administration of the dual N-type and L-type calcium channel blocker selective for the N-type calcium channel. In some embodiments, the concentration of IL-8 mRNA, IL-4, IL-10, or any combination thereof are measured in the cerebrospinal fluid or the plasma (e.g., the plasma) of the subject.

In some embodiments, the method further comprises determining that the subject has an abnormally high immunologic response, an abnormally high number or concentration of non-specific autoantibodies to sympathetic receptors, or both before administration of the dual N-type and L-type calcium channel blocker selective for the N-type calcium channel. In some embodiments, the method further comprises determining a reduction in immunologic response, a reduction in number or concentration of non-specific autoantibodies to sympathetic receptors, or both after administration of the dual N-type and L-type calcium channel blocker selective for the N-type calcium channel.

In some embodiments, the method further comprises determining autonomic dysregulation; an abnormally high number of sympathetic receptors; an abnormally high concentration of circulating catecholamines; sympathetic hyperactivity; or any combination thereof in the subject before administration of the dual N-type and L-type calcium channel blocker selective for the N-type calcium channel. In some embodiments, the method further comprises determining an improvement in autonomic functioning; a reduction in sympathetic receptors; a reduction in the concentration of circulating catecholamines; a reduction in sympathetic activity; or any combination thereof in the subject after administration of the dual N-type and L-type calcium channel blocker selective for the N-type calcium channel.

In some embodiments, the method further comprises determining an abnormally high central sensitization and neuroplasticity with increased substance P, bradykinin, and/or glutamate; an overexpression of spinal NMDA receptors; gabaminergic overactivity; or any combination thereof in the subject before administration of the dual N-type and L-type calcium channel blocker selective for the N-type calcium channel. In some embodiments, the method further comprises determining a reduction in central sensitization and neuroplasticity with increased substance P, bradykinin, and/or glutamate; an overexpression of spinal NMDA receptors; gabaminergic overactivity; or any combination thereof in the subject after administration of the dual N-type and L-type calcium channel blocker selective for the N-type calcium channel.

In some embodiments, the method further comprises determining peripheral oversensitization; an abnormally high concentration of spinal N-methyl-D-aspartate; an abnormally high concentration of spinal glutamate; upregulation in TNF-α and/or TRP-V1 activation; or any combination thereof in the subject before administration of the dual N-type and L-type calcium channel blocker selective for the N-type calcium channel. In some embodiments, the method further comprises determining a reduction in peripheral sensitization; a reduction in spinal N-methyl-D-aspartate; a reduction in spinal glutamate; a reduction in upregulation of TNF-α and/or TRP-V1 activation; or any combination thereof in the subject after administration of the dual N-type and L-type calcium channel blocker selective for the N-type calcium channel.

In some embodiments, the method further comprises determining abnormally high oxidative stress, a reduction in one or more oxidative stress markers, an abnormally high concentration of reactive oxygen species, immune system overactivation, or any combination thereof in the subject before administration of the dual N-type and L-type calcium channel blocker selective for the N-type calcium channel. In some embodiments, the method further comprises determining a reduction in oxidative stress, a reduction in one or more oxidative stress markers, a reduction in reactive oxygen species, a reduction in immune system activation, or any combination thereof in the subject after administration of the dual N-type and L-type calcium channel blocker selective for the N-type calcium channel. In some embodiments, the one or more oxidative stress markers are selected from nitrotyrosine, isoprostane, lactic dehydrogenase, uric acid, malondialdehyde, myeloperoxidase, oxidized low density lipoproteins, and S-glutathionylation of haemoglobin.

In some embodiments, the method further comprises determining that the subject has microvascular pathology and/or ischemia reperfusion injury before administration of the dual N-type and L-type calcium channel blocker selective for the N-type calcium channel. In some embodiments, the method further comprises determining that the microvascular pathology and/or ischemia reperfusion injury in the subject is treated, improved, or ameliorated after administration of the dual N-type and L-type calcium channel blocker selective for the N-type calcium channel.

In some embodiments, the method further comprises determining endothelial dysfunction in the subject before administration of the dual N-type and L-type calcium channel blocker selective for the N-type calcium channel. In some embodiments, endothelial function in the subject is improved after administration of the dual N-type and L-type calcium channel blocker selective for the N-type calcium channel.

In another aspect, disclosed herein is a method of treating eye pain or an eye disease or disorder in a subject in need thereof, comprising: (1) determining that the subject has TRP-v1 overactivation; and (2) administering a therapeutically effective amount of a dual N-type and L-type calcium channel blocker selective for the N-type calcium channel to the subject.

In some aspects, disclosed herein is a method of treating eye pain or an eye disease or disorder in a subject showing a clinical record of TRP-v1 overactivation in need thereof, comprising administering a therapeutically effective amount of a dual N-type and L-type calcium channel blocker selective for the N-type calcium channel to the subject.

In some aspects, disclosed herein is a method of treating eye pain or an eye disease or disorder in a subject in need thereof, comprising (1) administering a therapeutically effective amount of a dual N-type and L-type calcium channel blocker selective for the N-type calcium channel to the subject; and (2) determining a reduction of TRP-v1 activity in the subject.

In some aspects, disclosed herein is a method of treating eye pain or an eye disease or disorder in a subject in need thereof, the method comprising (a) determining that the eye pain or eye disease or disorder is associated with vasoconstriction or neuropathic pain; (b) determining that the subject has TRP-v1 overactivity; and (c) administering to the subject a therapeutically effective amount of a dual N-type and L-type calcium channel blocker selective for the N-type calcium channel.

In some aspects, disclosed herein is a method of treating eye pain or an eye disease or disorder in a subject showing a clinical record of TRP-v1 overactivation in need thereof, the method comprising (a) determining that the eye pain or eye disease or disorder is associated with vasoconstriction or neuropathic pain; and (b) administering to the subject a therapeutically effective amount of a dual N-type and L-type calcium channel blocker selective for the N-type calcium channel.

In some aspects, disclosed herein is a method of treating eye pain or an eye disease or disorder in a subject in need thereof, the method comprising (a) determining that the eye pain or eye disease or disorder is associated with vasoconstriction or neuropathic pain; (b) administering to the subject a therapeutically effective amount of a dual N-type and L-type calcium channel blocker selective for the N-type calcium channel; and (c) determining a reduction of TRP-v1 activity in the subject.

Additional methods and compositions for treating various diseases and disorders with a dual N-type and L-type calcium channel blocker selective for the N-type calcium channel can be found in WO 2021/178903 and WO 2022/115576, each of which is incorporated by reference herein in its entirety.

### Pharmaceutical Compositions and Formulations

In some aspects, disclosed herein is a pharmaceutical composition comprising a dual N-type and L-type calcium channel blocker selective for the N-type calcium channel, an agent that increases blood pressure, and optionally a pharmaceutically acceptable excipient.

In some aspects, disclosed herein is a pharmaceutical composition comprising a dual N-type and L-type calcium channel blocker selective for the N-type calcium channel; a therapeutic agent selected from the group consisting of: a beta blocker, an alpha agonist, a carbonic anhydrase inhibitor, a cholinergic, a prostaglandin, a prostamide, or a combination thereof; and optionally a pharmaceutically acceptable excipient. In some embodiments, the beta blocker is brimonidine or timolol. In some embodiments, the beta blocker is timolol. In some embodiments, the carbonic anhydrase inhibitor is Diamox or dorzolamide. In some embodiments, the cholinergic is pilocarpine. In some embodiments, the prostaglandin is latanoprost. In some embodiments, the prostamide is bimatoprost. In some embodiments, the therapeutic agent is selected from the group consisting of: brimonidine, timolol, Diamox, dorzolamide, pilocarpine, latanoprost, and bimatoprost. In some embodiments, the therapeutic agent is a beta blocker. In some embodiments, the therapeutic agent is timolol.

In some embodiments, the dual N-type and L-type calcium channel blocker selective for the N-type calcium channel is selected from the group consisting of: cilnidipine, Z-160, zicinotide, ralfinamide, CNV2197944, or pharmaceutically acceptable salts thereof.

In some embodiments, the dual N-type and L-type calcium channel blocker selective for the N-type calcium channel is selected from the group consisting of: cilnidipine, Z-160, ralfinamide, CNV2197944, or pharmaceutically acceptable salts thereof.

In some embodiments, the dual N-type and L-type calcium channel blocker selective for the N-type calcium channel is selected from the group consisting of: cilnidipine, Z-160, CNV2197944, or pharmaceutically acceptable salts thereof.

In some embodiments, the dual N-type and L-type calcium channel blocker selective for the N-type calcium channel is selected from the group consisting of: cilnidipine, Z-160, zicinotide, or pharmaceutically acceptable salts thereof.

In some embodiments, the dual N-type and L-type calcium channel blocker selective for the N-type calcium channel is cilnidipine or a pharmaceutically acceptable salt thereof.

In some embodiments, the dual N-type and L-type calcium channel blocker selective for the N-type calcium channel is CNV2197944 or a pharmaceutically acceptable salt thereof.

In some embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines.

In some embodiments, the pharmaceutical composition further comprises a non-steroidal anti-inflammatory drug. In some embodiments, the non-steroidal anti-inflammatory drug is aspirin.

In some embodiments, the dual N-type and L-type calcium channel blocker selective for the N-type calcium channel and/or sodium channel blocker can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in some embodiments 0.1-95%, in some embodiments 75-85%, and in some embodiments 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, ocular, parenteral, transdermal, intranasal, sublingual, neuraxial, or oral. In some embodiments, the dual N-type and L-type calcium channel blocker selective for the N-type calcium channel is administered intraocularly.

The pharmaceutically acceptable carriers, solvents, diluents, excipients, adjuvants and vehicles generally refer to inert, non-toxic solid or liquid fillers, diluents, or encapsulating material not reacting with the active ingredients of the invention.

A review of the considerations to be taken into account in the preparation of a pharmaceutical composition for topical ocular delivery, can be found in Bar-Ilan and Neumann, in Textbook of Ocular Pharmacology, Zimmerman et al eds., Lippencott-Raven 1997, which is incorporated by reference herein in its entirety.

In some embodiments, the dual N-type and L-type calcium channel blocker selective for the N-type calcium channel is administered topically. Topical routes of administration are preferably employed for providing the subject with an effective dosage of the dual N-type and L-type calcium channel blocker selective for the N-type calcium channel. In some embodiments, the dual N-type and L-type calcium channel blocker selective for the N-type calcium channel is administered as a solution or suspension. Dosage forms may include dispersions, suspensions, solutions, and the like. In some embodiments the pharmaceutical composition is delivered as eye drops. In some embodiments the pharmaceutical composition is delivered as a spray or mist. Ophthalmic spray applicators that can be used in this method of delivery are disclosed in, e.g., U.S. Pat. No. 4,052,985, which is incorporated by reference herein in its entirety.

In some embodiments, the composition is in the form of eye drops. In some embodiments, the eye drops comprises any combination of the following components: polyethylene glycol, light mineral oil, mineral oil, ascorbic acid, benzalkomium chloride, boric acid, dextrose, disodium phosphate, glycerin, glycine, hypromellose, magnesium chloride, potassium chloride, purified water, sodium borate, sodium chloride, sodium citrate, sodium lactate, edetate disodium, octoxynol-40, polyquaternium-1, polysorbate 80, sodium borate decahydrate, propylene glycol, hydroxypropyl guar, dimyristoyl phosphatidylglycerol, polyoxyl 40 stearate, sorbitan tristearate, sorbitol, polidronium chloride, carboxymethylcellulose, hyaluronic acid, Sodium Hydrogen Carbonate, L-Carnosine, N-Acetyl-L-Carnosine, Cysteine Ascorbate, Glutathione, Riboflavin Monophosphate, L-Taurine, pheniramine maleate, and naphazoline hydrochloride.

In some embodiments, the dual N-type and L-type calcium channel blocker selective for the N-type calcium channel reaches its target cells locally, for example, by direct contact or by diffusion through cells, tissue, or intracellular fluid. The dual N-type and L-type calcium channel blocker selective for the N-type calcium channel are administered and dosed in accordance with good medical practice, taking into account the clinical condition of the individual patient, the disease to be treated, scheduling of administration, patient age, sex, body weight, and other factors known to medical practitioners.

In some embodiments, the pH of the formulation is about pH 5 to about pH 8. For example, about pH 5 to about pH 7 or about pH 5 to about pH 6. In some embodiments the pH is about pH 5.9, about pH 6.15, about pH 6.25, about pH 6.3, about pH 6.5, or about pH 7.25. The compounds of the present invention can be administered topically to the surface of the eye. It should be noted that the compound is preferably applied as the compound or as pharmaceutically acceptable salt active ingredient in combination with pharmaceutically acceptable carriers, solvents, diluents, excipients, adjuvants and or vehicles. As disclosed herein the preferred method of delivery is topical application of an ophthalmic composition to the eye.

Liquid forms may be prepared for delivery in drops or a spray. The liquid compositions include aqueous solutions, with and without organic co-solvents, aqueous or oil suspensions, emulsions with edible oils, and similar pharmaceutical vehicles.

Compositions can also be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In some embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In some embodiments, the compositions take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In some embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In some embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosing and Regimens

In some embodiments, the dosage of the dual N-type and L-type selective calcium blocker (e.g., cilnidipine) is from about 1 mg to about 100 mg (e.g., from about 1 mg to about 50 mg, from about 1 mg to about 25 mg, from about 1 mg to about 15 mg, from about 1 mg to about 10 mg, from about 1 mg to about 5 mg, from about 1 mg to about 3 mg, from about 3 mg to about 35 mg, from about 5 mg to about 25 mg, from about 8 mg to about 28 mg, from about 12 mg to about 28 mg, from about 9 mg to about 21 mg, from about 15 mg to about 25 mg, from about 20 mg to about 30 mg, from about 22 mg to about 28 mg, from about 17 mg to about 23 mg, from about 8 mg to about 12 mg, about 8 mg, about 2 mg, about 4 mg, about 6 mg, about 8 mg, about 10 mg, about 12 mg, about 15 mg, about 18 mg, about 20 mg, about 22 mg, about 25 mg, about 30 mg, about 35 mg, about 40 mg, or about 50 mg). In some embodiments, the dosage of the dual N-type and L-type selective calcium blocker is from about 5 mg to about 25 mg. In some embodiments, the dosage of the dual N-type and L-type selective calcium blocker is from about 9 mg to about 21 mg. In some embodiments, the dosage of the cilnidipine is about 10 mg. In some embodiments, the dosage of the cilnidipine is about 20 mg. In some embodiments, the dosage of the cilnidipine is about 25 mg. In some embodiments, the dosage of the cilnidipine is about 30 mg. In some embodiments, the cilnidipine is administered topically. In some embodiments, the cilnidipine is administered topically to the eye. In some embodiments, the cilnidipine is administered orally.

In some embodiments, the dosage form of the cilnidipine comprises a concentration of from about 0.01% to about 50% (e.g., from about 0.01% to about 50%, about 0.01% to about 40%, about 0.01% to about 30%, about 0.01% to about 25%, about 0.01% to about 20%, about 0.01% to about 15%, about 0.01% to about 10%, about 0.01% to about 7%, about 0.01% to about 5%, about 0.01% to about 3%, about 0.01% to about 2%, about 0.01% to about 1%, about 0.25% to about 10%, about 0.25% to about 7%, about 0.25% to about 5%, about 0.25% to about 3%, about 0.25% to about 2%, about 0.25% to about 1%, about 10%, about 7%, about 5%, about 4%, about 3%, about 2%, about 1%, about 0.5%, about 0.25%, about 0.1%, or about 0.01%) by weight of cilnidipine. For example, the dosage form of the cilnidipine comprises a concentration of from about 0.01% to about 10% by weight of cilnidipine. For example, the dosage form of the cilnidipine comprises a concentration of from about 0.25% to about 5% by weight of cilnidipine. For example, the dosage form of the cilnidipine comprises a concentration of from about 0.01% to about 10% by weight of cilnidipine.

The foregoing dosages can be administered on a daily basis (e.g., as a single dose or as two or more (e.g., 2, 3, 4, or 5) divided doses) or non-daily basis (e.g., every other day, every two days, every three days, once weekly, twice weeks, once every two weeks, once a month).

In some embodiments of the methods disclosed herein, the dual N-type and L-type calcium channel blocker selective for the N-type calcium channel is administered orally, parenterally, transdermally, intranasally, sublingually, neuraxially, or ocularly. In some embodiments, the dual N-type and L-type calcium channel blocker selective for the N-type calcium channel is administered orally.

In some embodiments, the dual N-type and L-type calcium channel blocker selective for the N-type calcium channel and the at least one additional therapeutic agent are administered separately, sequentially, or simultaneously. In some embodiments, the dual N-type and L-type calcium channel blocker selective for the N-type calcium channel and the at least one additional therapeutic agent are administered separately. In some embodiments, the dual N-type and L-type calcium channel blocker selective for the N-type calcium channel and the at least one additional therapeutic agent are administered sequentially. In some embodiments, the dual N-type and L-type calcium channel blocker selective for the N-type calcium channel and the at least one additional therapeutic agent are administered simultaneously. In some embodiments, the du dual N-type and L-type calcium channel blocker selective for the N-type calcium channel and the at least one additional therapeutic agent are administered simultaneously as a fixed dosage form.

In some embodiments, each administration of the dual N-type and L-type selective calcium blocker and the at least one additional therapeutic agent is less frequent than the frequency of administering the dual N-type and L-type calcium channel blocker selective for the N-type calcium channel alone useful to treat the eye pain or eye disease or disorder.

In some embodiments, each administration of the dual N-type and L-type calcium channel blocker selective for the N-type calcium channel is separated by at least about 1 hour (e.g., about 1 to about 48 hours, about 1 to about 12 hours, about 12 to about 24 hours, about 2 to about 10 hours, about 4 to about 12 hours, about 6 to about 10 hours, about 10 to about 16 hours, about 14 to about 22 hours, about 16 to about 24 hours, about 24 to about 30 hours, about 30 to about 36 hours, about 36 to about 42 hours, about 40 to about 48 hours, at least about 2 hours, at least about 4 hours, at least about 6 hours, at least about 8 hours, at least about 12 hours, at least about 15 hours, at least about 20 hours, at least about 24 hours, at least about 36 hours, at least about 48 hours, at least about 72 hours, at least about 4 days, at least about 5 days, at least about 3 days, at least about 5 days, at least about 1 week, about 2 hours, about 4 hours, about 8 hours, about 12 hours, about 16 hours, about 20 hours, about 24 hours). In some embodiments, each administration of the dual N-type and L-type calcium channel blocker selective for the N-type calcium channel is separated by at least about 2 hours. In some embodiments, each administration of the dual N-type and L-type calcium channel blocker selective for the N-type calcium channel is separated by at least about 4 hours. In some embodiments, each administration of the dual N-type and L-type calcium channel blocker selective for the N-type calcium channel is separated by at least about 8 hours. In some embodiments, each administration of the dual N-type and L-type calcium channel blocker selective for the N-type calcium channel is separated by at least about 12 hours. In some embodiments, each administration of the dual N-type and L-type calcium channel blocker selective for the N-type calcium channel is separated by at least about 24 hours. In some embodiments, each administration of the dual N-type and L-type calcium channel blocker selective for the N-type calcium channel is separated by at least about 48 hours. In some embodiments, each administration of the dual N-type and L-type calcium channel blocker selective for the N-type calcium channel is separated by at least about 72 hours. In some embodiments, each administration of the dual N-type and L-type calcium channel blocker selective for the N-type calcium channel is separated by at least about 1 week. In some embodiments, each administration of the dual N-type and L-type calcium channel blocker selective for the N-type calcium channel is separated by about 4 hours. In some embodiments, each administration of the dual N-type and L-type calcium channel blocker selective for the N-type calcium channel is separated by about 4 to about 12 hours. In some embodiments, each administration of the dual N-type and L-type calcium channel blocker selective for the N-type calcium channel is separated by at least about 1 week.

In some embodiments, after administration (for example, after oral administration) of the dual N-type and L-type calcium channel blocker selective for the N-type calcium channel, the subject experiences gastrointestinal symptoms that are ameliorated by the consumption of food prior to administration of the dual N-type and L-type calcium channel blocker selective for the N-type calcium channel. In some embodiments, the subject consumes food up to about 6 hours before administering the dual N-type and L-type calcium channel blocker selective for the N-type calcium channel. For example, the subject consumes food up to about 5 hours, about 4 hours, about 3 hours, about 2 hours, about 1 hour, about 30 minutes, about 20 minutes, about 15 minutes, about 10 minutes, about 5 minutes, about 1 minute, about 30 seconds, or about 5 seconds before administering the dual N-type and L-type calcium channel blocker selective for the N-type calcium channel. For example, the subject consumes food concurrently with administering the dual N-type and L-type calcium channel blocker selective for the N-type calcium channel.

In some embodiments, the period of administration of the dual N-type and L-type calcium channel blocker selective for the N-type calcium channel is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, about 5 days to about 14 days, about 1 day to about 1 month, about 1 day, to about two weeks, at least about 1 month, 4 months, 5 months, 6 months, 7 months, 8 months, 9 months, 10 months, 11 months, 12 months, at least about 1 year, at least about 2 years, at least about 5 years, at least about 10 years, at least about 15 years, at least about 20 years, or longer. In some embodiments, the period of administration of the dual N-type and L-type calcium channel blocker selective for the N-type calcium channel is from about 1 day to about 1 month. In some embodiments, the period of administration of the dual N-type and L-type calcium channel blocker selective for the N-type calcium channel is from about 1 day to about two weeks. In some embodiments, the period of administration of the dual N-type and L-type calcium channel blocker selective for the N-type calcium channel is about two weeks. In some embodiments, the period of administration of the dual N-type and L-type calcium channel blocker selective for the N-type calcium channel is about 12 days. In some embodiments, the period of administration of the dual N-type and L-type calcium channel blocker selective for the N-type calcium channel is about one week. In some embodiments, the period of administration of the dual N-type and L-type calcium channel blocker selective for the N-type calcium channel is for at least about one month. In some embodiments, the period of administration of the dual N-type and L-type calcium channel blocker selective for the N-type calcium channel is for at least about one year. In some embodiments, a period during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or greater than 12 months. In some embodiments, a period during which administration is stopped is for up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 3 weeks, up to 4 weeks, up to 5 weeks, up to 6 weeks, up to 7 weeks, up to 8 weeks, up to 9 weeks, up to 10 weeks, up to 11 weeks, up to 12 weeks, up to 4 months, up to 5 months, up to 6 months, up to 7 months, up to 8 months, up to 9 months, up to 10 months, up to 11 months, or up to 12 months. In an embodiment, the dual N-type and L-type calcium channel blocker selective for the N-type calcium channel is administered to an individual for a period of time followed by a separate period of time. In another embodiment, the dual N-type and L-type calcium channel blocker selective for the N-type calcium channel is administered for a first period and a second period following the first period, with administration stopped during the second period, followed by a third period where administration of the dual N-type and L-type calcium channel blocker selective for the N-type calcium channel is started and then a fourth period following the third period where administration is stopped. In an aspect of this embodiment, the period of administration of the dual N-type and L-type calcium channel blocker selective for the N-type calcium channel followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or greater. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, the dual N-type and L-type selective calcium blocker is formulated to maintain the plasma level of the dual N-type and L-type selective calcium blocker in the subject at 10% or greater (e.g., 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, or 95% or greater) of the peak dual N-type and L-type selective calcium blocker plasma level for at least 6 hours (e.g., at least 8 hours, at least 12 hours, at least 16 hours, at least 20 hours, at least 24 hours, at least 36 hours, or at least 48 hours) after administration of the dual N-type and L-type selective calcium blocker. It is understood that the peak dual N-type and L-type selective calcium blocker plasma level is the highest plasma concentration of the dual N-type and L-type selective calcium blocker observed in the subject after administration of the dual N-type and L-type selective calcium blocker.

The invention will be further described in the following examples, which do not limit the scope of the invention described in the claims.

### EXAMPLES

### Example 1: Evaluation of Cilnidipine in the Treatment of Ocular Disease and Disorder

### Overview:

Study 1: A test agent to corneal sensitivity C57BI/6 mice will be evaluated.
C57Bl/6 mice, male/female, 6-10 weeks old
   n=20 mice total (n=5 mice/arm)
Experimental Design
   Baseline: Quantification of corneal sensitivity by aesthesiometer.
   Hour 0: Bilateral topical instillation of test agent.
   Hour 0 at 10 minutes post-instillation: Quantification of corneal sensitivity by aesthesiometer
   Hour 0 at 30 minutes post-instillation: Quantification of corneal sensitivity by aesthesiometer; Collection of eyes with tissue snap frozen and tube and tissue weights recorded for future bioanalytical analyses.
Study Arms
   Arm 1: Vehicle
   Arm 2: 0.1% Test Agent
   Arm 3: 0.25% Test Agent
   Arm 4: 1.0% Test Agent
Deliverables
   Compiled data of quantified corneal sensitivity
   40 mouse eyes individually snap-frozen and prepared for shipment on dry ice with tube and tissue weights recorded
      Study 2: Various test agents to reduce corneal permeability and maintain conjunctival goblet cell density in an acute mouse model of dry eye disease will be evaluated.
Dry Eye Disease, C57Bl/6 mice
   n=45 mice total
Experimental Design
   Days 1-5: Exposure to desiccating stress (Arms 2-5)
   Days 1-4: Topical instillation of vehicle, test agent, or positive control (3 IA volume), BID (Arms 3-5)
   Day 5: Quantification of corneal sensitivity by aesthesiometer; Quantification of corneal permeability; Enucleation of eyes for future histological study
Study Arms
   Arm 1: No desiccating stress (n=5)
   Arm 2: Untreated (n=10)
   Arm 3: 0.1% CsA-MiDROPS™ (positive control) (n=10)
   Arm 4: Vehicle (n=10)
   Arm 5: XXX % Test Agent (n=10)
Deliverables
   Quantification of corneal sensitivity on Day 5
   Quantification of corneal permeability in all eyes at Day 5
   Enucleation and collection of eyes for histological examination
      Study 3: Evaluation of A test agent to prevent retinal ganglion cell loss in a mouse model of optic nerve crush will be evaluated.
C57Bl/6 mice, female
   N=5 mice/arm (10 mice total)
Experimental Design
   Days 1-15: Bilateral topical instillation of vehicle or test agent, BID
   Day 3: Bilateral optic nerve crush
   Day 16: Quantification of corneal sensitivity by aesthesiometer; Pattern ERG recording; Enucleation of eyes for flat mount immunohistochemistry to label RGC cells with RBPMS and Tuj 1; optic nerves collected and fixed for future histological analysis
Study Arms
   Arm 1: Vehicle
   Arm 2: Test Agent #1
Deliverables:
   Quantification of corneal sensitivity on Day 5
   Quantification of RBPMS-positive cells in retinal flat mounts
   Quantification of n1 and p2 amplitudes from pattern ERG recordings on Day 14
   Representative images of Tuj 1 staining in retinal flat mounts
   20 mouse optic nerves fixed for future histological analysis

Research Design.

Phase I will involve developing an optimal formulation of cilnidipine ophthalmic drug product for exploring its preliminary safety and efficacy in the treatment of ocular pain. The solubility and stability of cilnidipine in several formulations will be evaluated using different commercial excipients and delivery vehicles. Following these preliminary physicochemical explorations, a decision will be made regarding the optimal formulation for the preparation of the sterile drug product. Preparation standards will be at Good Laboratory Practices (GLP) level. A preservative will be added to the cilnidipine ophthalmic product to be evaluated. The formulations will be evaluated for desirable solubility and stability. Topical solutions of cilnidipine will be prepared in a range of concentrations. Tolerability/toxicity testing will be performed at different concentrations. The different concentration of cilnidipine solution will be tested using aesthesiometry to quantify corneal sensitivity (topical anesthesia) in a murine model at baseline and at selected intervals post-instillation of the test agent. Ocular tissue distribution and penetrance will be analyzed by samples samples of ocular tissue (cornea, retina, meibomian gland, trabecular network) by tissue pharmacokinetic (PK) analysis. A validated method for determining cilnidipine concentrations in tissues was developed using LC/MS. A range of two concentrations of cilnidipine drug product including the highest concentration that produces no topical anesthesia, will be chosen for further testing in dry eye and nerve crush injury efficacy models. Test eyes will be collected and frozen for future tissue analysis.

Specific Aim 1: Develop a New Topical Formulation of Cilnidipine. [2 Months].

The program will start by developing topical ophthalmic formulations of cilnidipine in several concentrations. Initial experiments in Phase 1 will first determine, amongst a variety of lipid excipients, which excipient provides the optimal solubilization and tissue delivery of the active agent cilnidipine. Cilnidipine active pharmaceutical ingredient (API) will be solubilized and obtained from TargetMol, evaluating the suitability of specified commercial excipients and delivery vehicles to determine which one provides most favorable solubility and stability characteristics and then prepare topical formulations of cilnidipine in this vehicle in several concentrations ranging from 0.1% to 4%. The formulations will be observed for stability and the cilnidipine will be prepared in an excipient and preservative formulation which has already been approved by the FDA in ophthalmic topical eyedrops that are commercially available.

### Task 1.1: Development of Different Formulations of Cilnidipine.

The cilnidipine drug product physical and chemical characteristics will be reviewed from published databases such as PubChem, a determination will be made as to which of several lipid solutions are most likely to be successful excipients for cilnidipine ophthalmic drug product. Multiple formulation vehicles suitable for ophthalmic delivery with targeted strengths between 0.1-4% will be assembled. Initial experiments will start with a concentration of 1% cilnidipine, and will use lipid-based excipients, used clinically in topical eye-drop formulations. Additional concentrations will then be explored. Optionally, liposomes containing cilnidipine will be made as part of the solubilization process, since this is a standard technique for the delivery of lipid based ophthalmic products.

### Task1.2: Evaluation of Different Formulations.

The above formulations will be evaluated for solubility and stability. If multiple formulations result in suitable end products that are candidates for further evaluation, the investigative team will decide which formulation to advance based on clinical experience with the lipid formulation excipients and which formulation is likely to be most suitable for clinical study and commercialization.

### Task 1.3: Determination of Most Favorable Formulation.

The most favorable formulation will be determined based on solubility and stability characteristics, as described previously.

Task 1.4: Development of Topical Formulation of Cilnidipine in Selected Formulation at Concentrations Ranging from 0.1% to 4%.

Concentrations of the cilnidipine drug product will be evaluated at the following concentrations: cilnidipine 0.1%, 0.25%, 0.5%, 0.75%, 1.0%, 1.5%, 2.0%, and 4.0%.

Innovative approach/methodology.

Existing methodologies and models to evaluate a calcium channel antagonist which has innovative properties on relevant cell pathways that have been discovered will be evaluated. Ocular Coherence Tomography (OCT) of the cornea may be used when examining for efficacy and safety of the drug product in regard to epithelial defects and tissue effects that are not picked up with conventional fluorescein staining.

An initial set of experiments to determine whether the solution causes surface anesthesia of the animal eye and to determine the maximal concentration that does not produce topical anesthesia of the eye surface will be performed.

### Task 2.1: Determine Corneal Sensitivity at Selected Timepoints and Concentrations by Aesthesiometry.

Corneal sensitivity will be quantified by aesthesiometry, the current established method for measuring the level of corneal sensitivity, in 20 C57BL/6 male and female mice (40 eyes), 6-10 weeks old, at baseline and at 10 minutes and 30 minutes post-instillation of the test agent. Four study arms (n=5 mice/arm) will be used: Arm 1: Vehicle; Arm 2: 0.1% cilnidipine solution test agent; Arm 3: 0.25% test agent; and Arm 4: 1.0% test agent. Bilateral topical instillation will be performed. Compilation of data results of quantified corneal sensitivity will be provided. Aesthesiometry will be conducted using a Cochet-Bonnet aesthesiometer. The highest concentration solution of cilnidipine that results in no decrease in corneal sensitivity will be selected for further testing. Doses bracketing this dose (one lower in concentration and the other just above in terms of concentration) will be noted and sufficient quantities of these concentrations will be produced for further possible investigation.

### Aim 2.2: Collection and Freezing of Eyes for Future Bioanalytical Analyses.

Following determination of corneal sensitivity, the mice will be sacrificed and the eye and surrounding tissue preserved. Collection of the 40 mouse eyes, with tissue individually snap frozen, will be performed and prepared for shipment on dry ice, with tube and tissue weights recorded, for future analyses. These analyses will include tissue penetrance assays to determine whether the drug is present in the posterior chamber of the eye and in what concentration and also whether the drug is present in the retinal ganglia and at what concentration and finally whether the drug is detectable at the optic nerve and in what concentrations. Eyes will also be examined for any surface changes or toxicities, epithelial defects, or other changes.

### Interpretation of Results.

If cilnidipine is found to produce topical anesthesia of the eye at all concentrations, then toxicity evaluations will be done. If cilnidipine is found to be toxic to the eye at all concentrations, this project will be deemed to be a failure and no further studies or expenditure of funds beyond paying for what has already been accomplished will be done. If a suitable concentration (one in the range of concentrations being studied) appears to not decrease corneal sensitivity, then examination of whether any demonstrable ocular toxicity results from this concentration can be detected. If there is evidence of ocular toxicity at this concentration, the highest dose of cilnidipine tested without evidence of ocular toxicity will be advanced to the efficacy studies. If a selected concentration of cilnidipine produces a decrease in corneal sensitivity but no evidence of ocular toxicity, this dose will also be studied in the efficacy studies to test for whether it appears to provide relief in ocular pain models. Should these studies demonstrate that the cilnidipine product is effective in models of ocular pain, the concentration will be further evaluated in toxicity studies with longer term dosing (e.g., 5 days) and use of optical coherence tomography in addition to visual inspection to determine whether any toxicity hallmarks can be detected.

Specific Aim 3: Perform Pharmacokinetic (Pk) and Toxicity Studies. [4 Months].

Aim 3 will be evaluated to determine whether the topical dose of the drug produces any appreciable penetration of drug levels into specified ocular tissues, and at what concentration and for how long. The selected concentration will then be dosed on a regular schedule to a small group of C57BL/6 mice and their corneas, retinas, meibomian glands and optic nerves will be harvested to determine whether the drug dosed topically develops any appreciable penetration or drug levels in these tissues. Samples will be evaluated by PK analysis. Any apparent toxicity associated with the application of cilnidipine at regular intervals at this concentration over time in the animal eyes will be evaluated.

### Task 3.1. PK STUDIES.

The selected cilnidipine concentration will be dosed on a regular schedule to a small group of C57BL/6 mice and their corneas, irises, ciliary bodies, aqueous humor, vitreous humor, retinas, optic nerves, and blood plasma will be harvested to determine whether the drug dosed topically produces any appreciable penetration of drug levels in these tissues. PK analysis will be evaluated.

### Task 3.2. Tolerability and Toxicity Study.

Tolerability or toxicity effects associated with the application of cilnidipine at regular intervals at the selected concentration over time in the animal eyes will be evaluated. The presence of any corneal surface abnormalities, general adverse health effects, or adverse effects on body weight after test drug administration will be evaluated.

The efficacy of the drug in two animal disease models will be evaluated:
(1) Scopolamine-Induced Dry Eye Disease model.
(2) Optic Nerve Crush Injury model of neuropathic pain.

### Task 4.1. Evaluate the Effect of Cilnidipine Topical Solution Compared to Controls on Corneal Permeability, Pain Response, and Conjunctival Goblet Cell Density in a Mouse Model of Dry Eye Disease.

The efficacy of cilnidipine in a scopolamine-induced mouse model of dry eye disease using 45 male and female C57BL/6 mice. Temperature and humidity will be controlled while the animals are exposed to constant air flow and receive daily systemic injections of scopolamine. Five arms will be used. Arm 1: No desiccating stress (n=5); Arm 2: Untreated (n=10; Arm 3: 0.1% CsA-MiDROPSTM (positive control) (n=10); Arm 4: Vehicle (n=10); and Arm 5: XXX % cilnidipine solution (n=10). The mice will be exposed to desiccating stress (Arms 2-5) on Days 1-5. Topical instillation of vehicle, test agent, or positive control (Arms 3-5) (311.1 volume), BID, will occur on Days 1-4. Mice will then be euthanized, and On Day 5, corneal permeability and conjunctival goblet cell density will be quantified. Ocular pain will be assessed with aesthesiometry using Von-Frey filaments and measuring response. Oregon green dextran (OGD) staining and photography under fluorescence excitation will be used to evaluate corneal epithelium integrity, and PAS staining, photography, and software will be used for conjunctival goblet cells quantification. Following the experiments animal eyes will be enucleated for later histological study.

### Task 4.2: Evaluate the Effect of Cilnidipine Topical Solution Compared to Controls for Prevention of Retinal Ganglion Cell Loss and Pain Response in a Mouse Model of Optic Nerve Crush.

The efficacy of cilnidipine to prevent retinal ganglion cell (RGC) loss in an optic nerve crush (ONC) model using 10 female C57BL/6 mice will be evaluated. ONC will be performed on anesthetized mice by exposing the optic nerve through the bulbar conjunctiva and transiently pinching the nerve just behind the globe using self-closing forceps. Two arms will be used: Arm 1: Vehicle; and Arm 2: Test Agent, with 5 mice per arm. The mice will receive bilateral topical instillation of either vehicle or test agent, BID, on Days 1-15. On Day 3, bilateral optic nerve crush will be performed. Quantification of n1 and p2 amplitudes from pattern ERG recordings will be provided on Day 14. Ocular pain will be assessed with aesthesiometry using Von-Frey filaments and measuring response. Enucleation of eyes for retinal flat mount immunohistochemistry will be performed to label RGC cell viability assessed by quantification of RBPMS-positive cells, and for labeling of representative images of TUJ1 staining to assess density and integrity of RGC axons proximal to the crush site. Twenty mouse optic nerves will be collected and fixed for future histological analysis.

### Potential Technological Challenges and Alternatives.

An initial potential challenge will be the inability to solubilize cilnidipine in a lipid-based excipient medium. Should this occur with standard excipient solutions as described, liposomes of enclosed cilnidipine may be developed as this technique has been successful in this scenario with other agents.

A second potential challenge is in the documentation of toxicity/safety issues. Our response, after verifying that these seem drug-related, will be to evaluate lower concentrations of the agent to a point at which these issues do not manifest themselves. If these are seen with the lowest concentration of drug being evaluate (0.1% cilnidipine), then the project will be terminated.

A third area of weakness is that the evaluation may show drug penetration into a target tissue through PK sampling, it may not be known whether an effective concentration of the drug to demonstrate efficacy has been reached. However, from a practical standpoint, efficacy will be evaluated at several concentrations and until efficacy is demonstrated.

A fourth area of weakness is that the program only evaluates ocular pain in two models, and there might be additional possible effects of the drug that are not being evaluated, such as the activity of cold receptors in the eye. Should the program prove positive, the study may be expanded to other models to determine the full range of beneficial effects of cilnidipine topical ophthalmic solution.

### Statistical Analysis.

Data will be recorded in a secure database and will be analyzed. Each experiment contains untreated and vehicle only treated animals with a minimum of 5 animals per testing group. Statistical analysis will be performed (student T test) for significance of effect in treated versus control animals. A final CSR will be prepared on each of the tested protocols.

### Milestones for Transitioning to Phase 2:

1) Efficacy is demonstrated in the dry eye disease model on any clinically meaningful or implicated endpoint.
2) Efficacy is demonstrated in the nerve crush injury model on any clinically meaningful or implicated endpoint.
3) Safety is demonstrated in the corneal aesthesiometry evaluations and toxicity measurements.
4) PK sampling reveals concentration of the drug in optic nerve or retina tissues.

### Example 2. Two-Phase, Phase 2 Study, Assessing the Safety of Cilnidipine in SSc-RP and Dose Effect of a Combination of Cilnidipine and Tadalafil

Introduction: A two-phase, phase 2 study, evaluation the safety of cilnidipine in SSc-RP and determination of whether a dose effect existed for cilnidipine at two doses, and whether addition of tadalafil to these doses complemented efficacy and its effect on safety.

### Summary of Methods and Protocol:

### Part A

Part A of the trial employed a dose-finding, parallel arm design. The intention was to include six patients in each of the six arms. In this part, the objective was to determine the appropriate dosage of cilnidipine and tadalafil, as well as their combination, for further evaluation.

### Part B

Part B, is designed as a prospective, double-blind, randomized, placebo-controlled, two-way crossover trial. It aims to enroll a total of 72 patients, with a statistical power of over 80%. The primary goal of Part B is to assess whether cilnidipine has a significant effect (>25% reduction) on the weekly frequency of Raynaud's episodes in patients experiencing more than one attack per day during a two-week screening period.

### Interventions

The study evaluated the following interventions: cilnidipine at doses of 10 mg and 20 mg, tadalafil at a dose of 5 mg, administered once daily, as well as placebo.

### Medication Dispensation and Diary Maintenance

Study medication and matching placebo were provided to patients in kits at the time of randomization, following the screening process. Participants were instructed to maintain a daily electronic diary using a cell-phone based case report form (CRF). If necessary, patients were allowed to supplement the electronic diary with a paper record.

### Treatment and Assessment

Patients received the assigned intervention for a duration of two weeks. Subsequently, they returned to the clinic for assessments. After treatment discontinuation, patients were followed up for safety purposes.

### Concomitant Medications

Throughout the study, patients were permitted to continue their current stable doses of medications prescribed for the management of Raynaud's and other concurrent conditions.

### Study Endpoints

The study evaluated multiple endpoints, including the weekly frequency of Raynaud's attacks (primary endpoint), Raynaud Condition Score (RCS), pain severity, attack duration, Scleroderma Health Assessment Questionnaire (SHAD), Patient-Reported Outcome (PRO), UCLA Gastrointestinal Tract (GIT) 2.0 assessment for gastrointestinal dysfunction, endothelial function assessed by Endo-PAT, thermography, and pharmacokinetics (PK).

### Detailed Protocol:

### Detailed Summary

A randomized, double-blind, placebo-controlled phase 2a study to assess the safety and efficacy of cilnidipine (10 mg and 20 mg) alone and in combination with 5 mg tadalafil in participants with diagnosis of secondary Raynaud's disease, also referred to as reconnoiter-1: randomized evaluation of the benefit of cilnidipine dose on the nature, observational indices, temperature changes, and overall effect in secondary Raynaud's disease.

### Overall Study Design

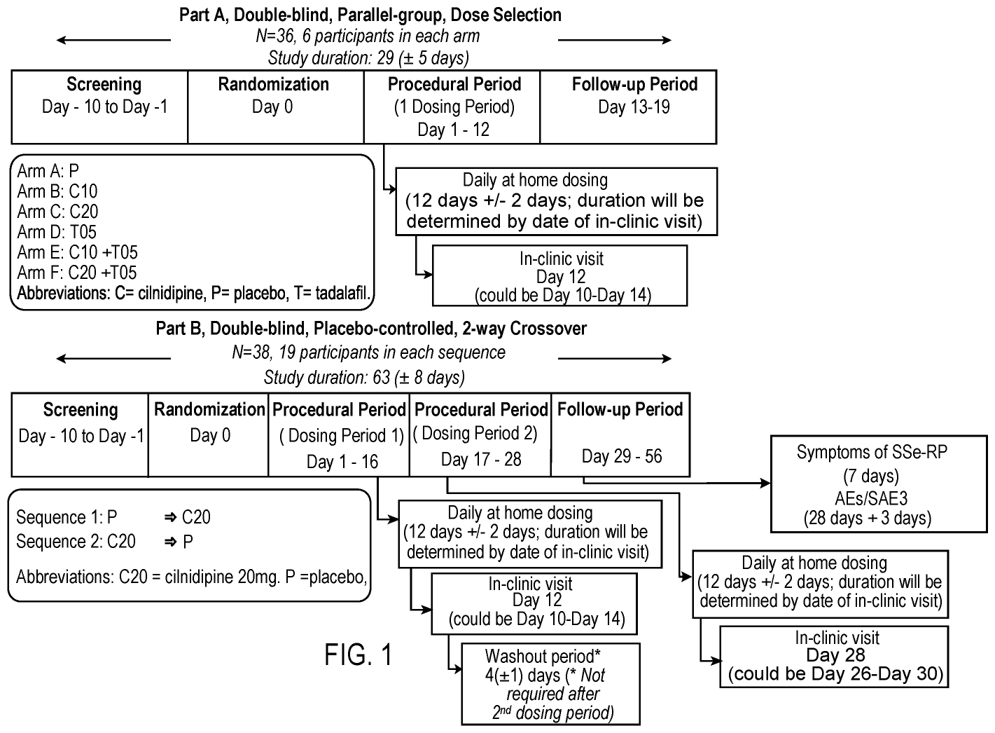

A schematic of the study design is provided in FIG. 1. This is a randomized, double-blind, placebo-controlled Phase 2a study to assess the safety and efficacy of cilnidipine alone and in combination with tadalafil, in participants who have frequent attacks of secondary RP mostly resulting from SSc. Oversight for the study will be provided by a DSMB.

Participants will undergo a screening period beginning up to 10 days prior to randomization. The initial screening and capacity will be conducted via phone at the start of the screening period with eligibility finalized prior to randomization on Day 0. Participants will be required to provide informed consent in a 2-step process at screening (commencement of diary use will be considered implied consent for the screening period) and at randomization (Day 0) before undertaking any study-specific procedures or assessments. Only participants who meet all of the inclusion and none of the exclusion criteria will be randomized.

### The Study Consists of Two Parts.

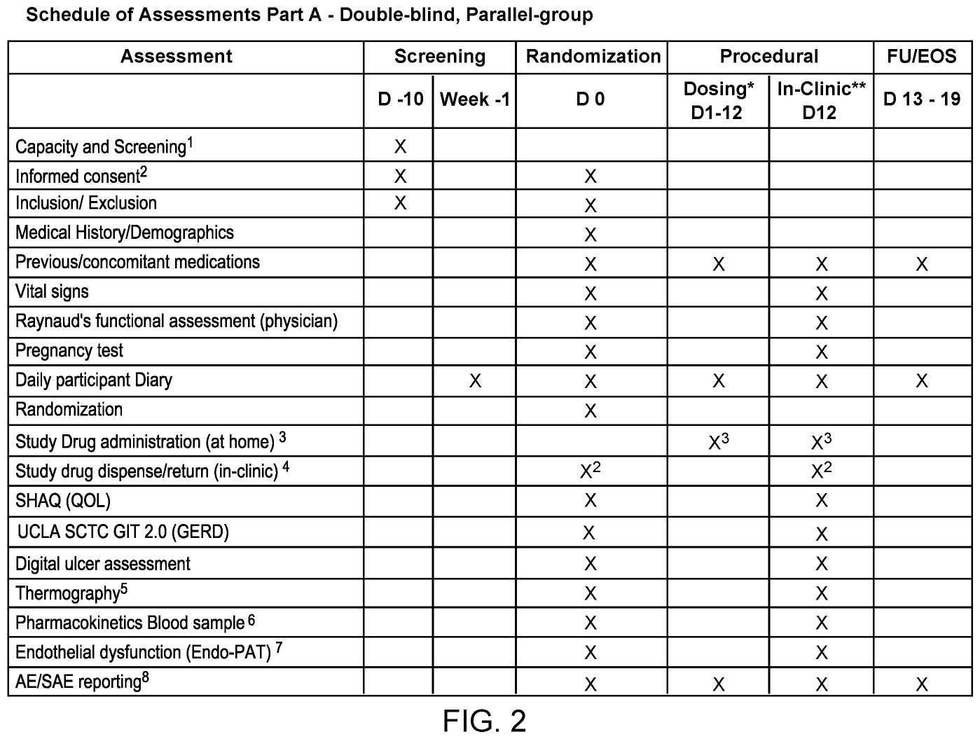

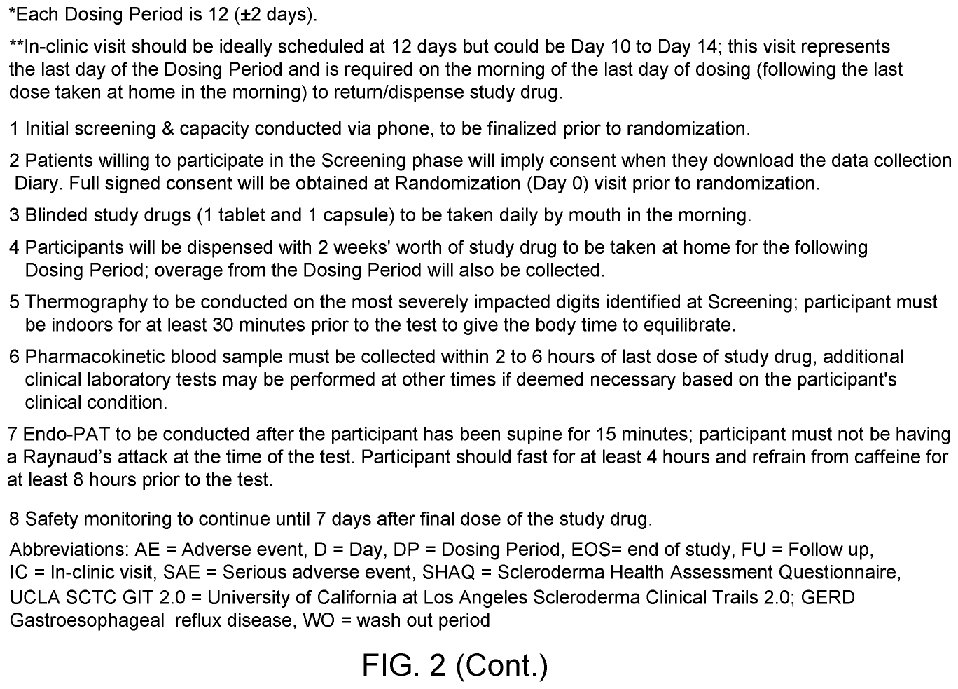

Part A—double-blind, placebo-controlled, parallel-group, dose selection, will assess the safety and efficacy of two doses of cilnidipine (10 mg and 20 mg), alone and in combination with tadalafil. A total of 36 participants will be randomized to one of six prespecified treatment arms. Refer to FIG. 1 for treatment arms in Part A of the study. Dosing last for 12 (±2) days in which participants will self-administer daily doses of assigned treatment in the morning. Each participant will take one capsule and one tablet to blind the active therapy being received. Study visits and assessments will occur as delineated in the SoA presented in FIG. 2.

The data from Part A of the study will be reviewed by an unblinded DSMB prior to selecting the cilnidipine dose and confirming the sample size estimates for the randomized double-blind phase (Part B). The first review will occur after approximately 50% of participants have completed the study.

The data obtained and reviewed from 27 participants enrolled in Part A was sufficient to 1) confirm safety, tolerability, and potential efficacy of cilnidipine in patients with SSc-RP and 2) select the cilnidipine dose (20 mg) for Part B for continued evaluation of efficacy, safety, and tolerability. The Committee also agreed that the co-administration of tadalafil with cilnidipine did not provide significant additional benefit to the higher dose of cilnidipine chosen and may add some minor adverse events; therefore, the Committee recommended modifying Part B to a 2-way crossover design (CIL 20>placebo, placebo>CIL 20).

Part B—Double-blind, placebo-controlled, 2-way crossover will assess the safety and efficacy of cilnidipine 20 mg (the dose selected in Part A). A total of 38 participants (19 in each sequence) with a diagnosis of SSc-RP will be randomized into one of two prespecified treatment sequences in a 2-way crossover design.

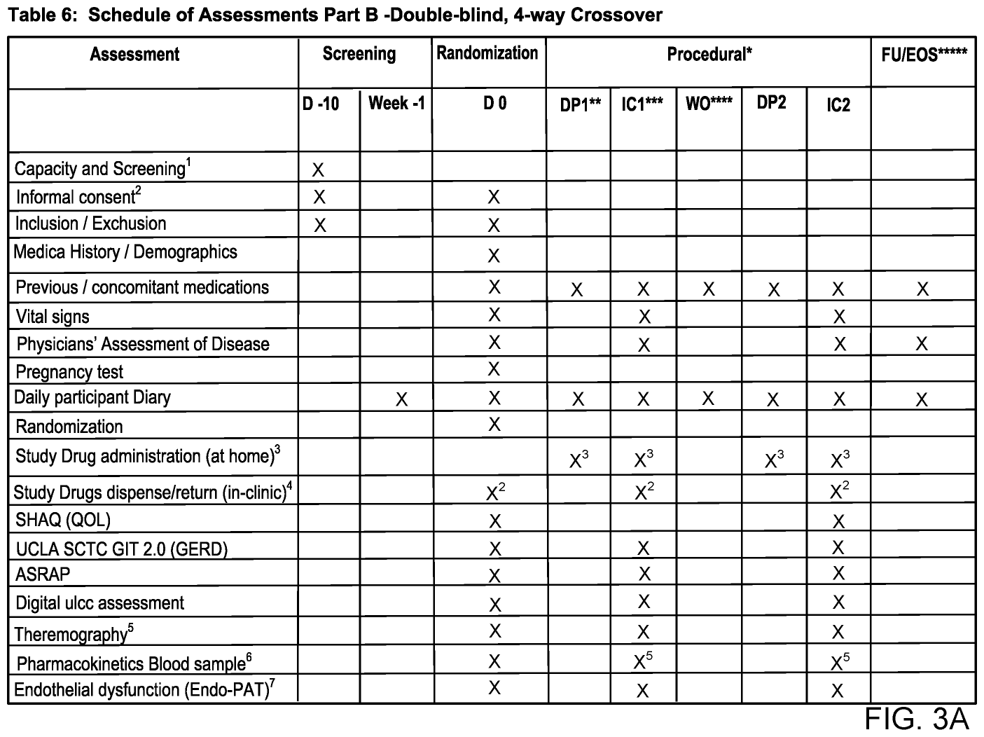

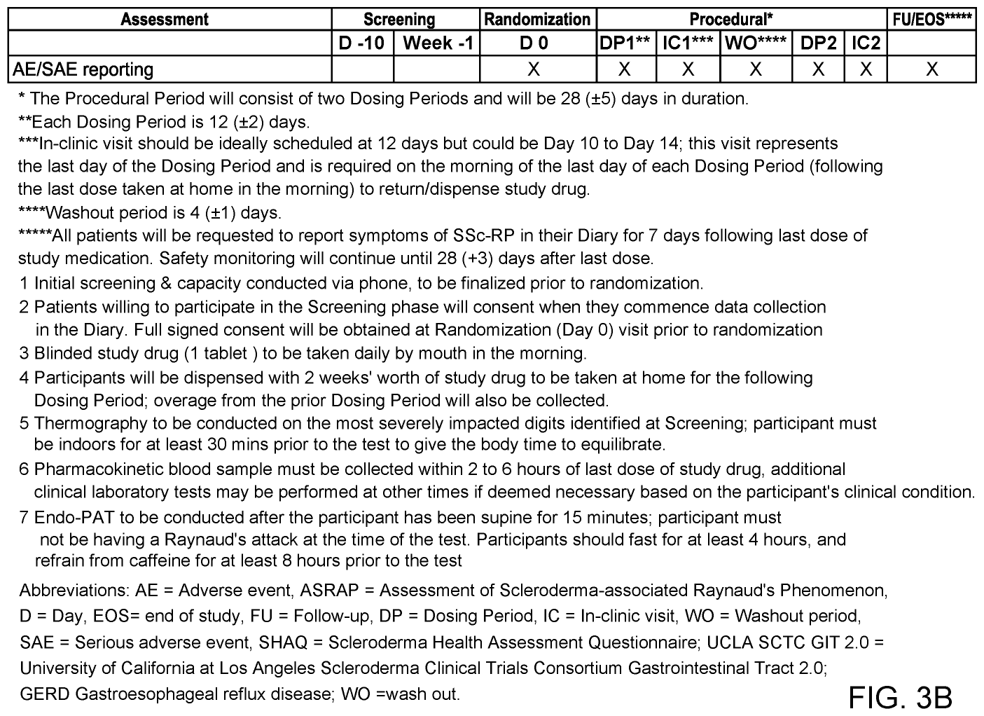

Refer to FIG. 1 for treatment sequences in Part B of the study. Each participant will undergo two dosing periods in which they will receive a different treatment each dosing period followed by a 4 (±1) day washout period. Each dosing period will last for 12 (±2) days in which participants will self-administer daily doses of assigned treatment in the morning. At all dosing periods, each participant will take one tablet to blind the active therapy being received. Study visits and assessments will occur as delineated in the SoA presented in FIGS. 3A and 3B.

For both Part A and B of the study, participants are required to visit the clinic on last day of each dosing period (i.e., day 10 to 14) to return/dispense study drug and conduct in person study assessments. Participants will be dispensed with 2 weeks' worth of study drug to be taken at home for the following dosing period; overage from the prior dosing period will also be collected.

Patients will be assessed for the occurrence of efficacy endpoints for each dosing period via the patient reported diary and the in-clinic visit. Safety information will be collected from randomization until patient follow-up is complete (28+3 days after the last dosing period) and assessed for each dosing period.

### Dose Selection, Sample Size Confirmation and Safety Oversight

The role of the DSMB, will be set out in a DSMB Charter. The DSMB will conduct a review of the efficacy and safety data from Part A of the study. The first review will occur after approximately 50% of the participants have completed the study. Subsequent reviews will occur as needed prior to commencement of Part B of the study to assess the risk: benefit of cilnidipine (10 mg and 20 mg), alone and in combination with tadalafil.

Following review of the efficacy and safety data from Part A, the DSMB will make the following recommendations:
1. Select the dose of cilnidipine (10 mg or 20 mg) to be studied in Part B of the study
2. Confirm the sample size estimates for Part B of the study.

Serious adverse events will be monitored (by the DSMB in Part A and Drug Safety Officer in Part B) on an ongoing basis throughout the study.

Efficacy and safety data from the 27 participants who completed Part A was included in the analyses. The benefit/risk profile demonstrated and recommended that the study proceed directly to Part B (powered crossover phase) without full completion of Part A. That the data from Part A is sufficient to 1) confirm safety, tolerability, and potential efficacy of cilnidipine in patients with SSc-RP and 2) select the cilnidipine dose (20 mg) for Part B for continued evaluation of efficacy, safety, and tolerability. The Committee also agreed that the co-administration of tadalafil with cilnidipine did not provide significant additional benefit to the higher dose of cilnidipine chosen and may add some minor adverse events; therefore, the Committee recommended modifying Part B to a 2-way crossover design (CIL 20>placebo, placebo>CIL 20).

Participants will receive study treatment (cilnidipine 20 mg or placebo tablet) in a blinded fashion. Each dosing period will last for 12 days (±2 days) in which participants will take daily doses of assigned treatment in the morning. The schematic for dosing is presented for Part A (Table 51) and Part B (Table S2) below and FIG. 1.

TABLE S0
Part A Demographic data

| | Pooled | All | |
|---|---|---|---|
| | Cilnidipine | Cilnidipine | Placebo |
| | only (n = 7) | (n = 16) | (n = 4) |
| Mean Age (SD) | 59.9(13.8) | 61.8 (11.1) | 49(7.8) |
| SSc Diagnosis | 86% | 88% | 100% |
| Presence of GI sx | 57% | 81% | 75% |
| Hx Diabetes | 14.3% | 12.5% | 25% |
| Hx CAD | 0% | 0% | 0% |
| Hx HTN | 0% | 0% | 0% |
| Abnl ANA | 100% (n = 6) | 92%(n = 13) | 75% |
| CRP/ESR levels elevated | 0% | 0% | 33% (n-3) |
| Smoking Hx | 28.6% | 31% | 75% |

TABLE S1
Part A, Double-blind, Parallel-group, Dose Selection

| Arm | N | Treatment |
|---|---|---|
| A | 6 | P |
| B | 6 | C10 |
| C | 6 | C20 |
| D | 6 | T05 |
| E | 6 | C10 + T05 |
| F | 6 | C20 + T05 |

Abbreviations: C = cilnidipine, P = placebo, T = tadalafil, N = number of participants, C10 = C 10 mg, C20 = C 20 mg; T05 = T 5 mg.

TABLE S2
Part B, Double-blind, Placebo-controlled, 2-way crossover

| Sequence Number | N | Dosing Period 1 | Dosing Period 2 |
|---|---|---|---|
| 1 | 19 | P | C20 |
| 2 | 19 | C20 | P |

Abbreviations: C20 = cilnidipine 20 mg, P = placebo, N = number of participants

Number of Participants (Planned): Up to 65 participants will be enrolled in this study: 27 (revised from 36 based on DSMB recommendation) in the parallel-group dose selection phase (Part A) and 38 in the 2-way crossover phase (Part B). Participants who complete Part A without any major protocol deviations or compliance issues will be invited to participate in Part B. Participants would need to consent and meet all eligibility criteria again in order to be randomized into Part B. Dropouts will not be replaced.

### Diagnosis and Main Criteria for Inclusion:

Participants aged 18-90 years and diagnosed with severe secondary Raynaud's disease (Raynaud's Condition Score [RCS]≥40 and at least a 2-phase color change in fingers of pallor, cyanosis, and/or reactive hyperemia in response to cold exposure or emotion) mostly resulting from SSc and exhibiting regular and frequent RP attacks (averaging at least one attack per day) during the screening period.

### Investigational Product, Dosage and Mode of Administration:

### Cilnidipine

For Part A, Cilnidipine 10 mg and cilnidipine 20 mg for oral administration, will be provided to the site in cartons containing 16 tablets sealed in blister packs. For Part B, Cilnidipine 20 mg (the dose selected in Part A) will be provided to the site in cartons containing 16 tablets sealed in blister packs.

### Tadalafil

For Part A, Tadalafil 5 mg, for oral administration will be over encapsulated (and back filled with inert capsule filler consisting of only maize starch and pre-gelatinized maize starch, so that the internal tablet cannot be detected) and provided in bottles containing 16 capsules to the site. Tadalafil will not be provided for Part B.

Medications will be dispensed during the preceding in-clinic study visit for self-administration by the participant once daily in the morning for a 12 (±2) day period. The duration of the Dosing period will be confirmed by the study staff when the in-clinic study visit is scheduled.

### Investigational Plan

### Study Rationale

No cure exists for patients suffering with SSc. The exact cause is unknown, however SSc is thought to result from a combination of factors, including autoimmune, genetic, and environmental triggers. Symptoms can include painful episodes in the extremities with color changes of the fingers or toes (e.g., RP), digital ulcers, skin thickening or hardening, capillary changes (assessed using nailfold capillaroscopy), swelling of the hands or legs, and general pain. Available therapies only provide symptomatic treatment with limited efficacy and safety. New treatments are needed to better manage the symptoms and directly address the underlying disease processes of SSc-RP. This study will evaluate the efficacy and safety of cilnidipine (Profervia®) monotherapy compared to placebo for the treatment of patients with secondary RP primarily due to SSc; the study will also include evaluation of combination therapy of cilnidipine and tadalafil to determine if the combination of cilnidipine with a low dose of the PDE5 inhibitor tadalafil will provide additional or synergistic benefits for the study patients.

The pharmacology and safety profile of cilnidipine make it a potentially more efficacious and safe treatment for SSc than the currently available medications, with the added potential benefits of diminishing the major symptoms of the disease including improvement of Raynaud's attacks (reduction of frequency and severity), as well as possibly addressing and improving the underlying pathologic processes contributing to disease progression, including fibrosis and endothelial/vascular dysfunction. It is further anticipated that the pharmacokinetic profile of cilnidipine make it better suited for the treatment of SSc-RP patients than other currently approved CCBs, since peak blood levels of the drug after oral dosing are reached within 2 hours, which is more rapidly than other CCBs.

### Dosage Rationale

### Cilnidipine

Cilnidipine is approved for the treatment of hypertension in Japan, India, China, and South Korea in doses of 5 mg up to 20 mg. It is taken orally, once a day, usually in the morning. First approved in Japan in 1995, there is greater than 25 years of safety experience at these doses.

Cilnidipine's pharmacokinetics, with a more rapid time to achieve maximal plasma concentrations (2 hour Tₘₐₓ) than the CCBs amlodipine (10 hours) or extended release nifedipine (3 hours), may increase cilnidipine's suitability for treating Raynaud's symptoms in SSc patients compared to other CCBs, in that these symptoms commonly occur early in the day. Cilnidipine is also dosed once daily compared to nifedipine's three times a day dosing schedule.

### Duration of Treatment:

Part A, Double-blind, Parallel-group, Dose Selection: Participants enrolled will participate for up to 29 (±5) days:
Screening: up to 10 days (−3 days)
Treatment: 12 (±2) days
Follow-up: 7 days (+3 days)

Part B, Double-blind, Placebo-controlled, 2-way crossover: Participants enrolled will participate for up to 63 days (±8 days):
Screening: up to 10 days (−3 days)
Treatment: 28 days (two days each lasting for 12 (±2) days, separated by a 4 (±1) day washout period)
Follow-up: 28 days (+3 days)

Reference Therapy, Dosage, and Mode of Administration:
Placebo: Placebo tablets (matching cilnidipine) and placebo capsules (matching tadalafil, Part A only), for oral administration, will be provided to the site.

### Study Objectives and Endpoints

### Objectives

### Primary Efficacy Objectives:

The primary efficacy objective of the study is to evaluate the effect of cilnidipine on the frequency of weekly RP attacks compared with placebo in participants with SSc-RP.

### Secondary Efficacy Objectives:

The secondary efficacy objective of the study is to evaluate the effect of cilnidipine on all the clinical features of SSc-RP, including symptoms and disability associated with SSc in addition to RP.

### Safety Objectives:

The safety objective of the study is to evaluate the safety of cilnidipine compared to placebo in participants with SSc-RP.

### Exploratory Objectives:

To assess the impact of treatment on 1) endothelial function and 2) severity and impact of Raynaud's phenomenon, in participants with SSc-RP

To evaluate the efficacy and safety of combination therapy (cilnidipine 10 mg and 20 mg, in combination with tadalafil) on all efficacy and safety endpoints (Part A only)

### Primary Efficacy Endpoint:

Percentage change from baseline in frequency of weekly RP attacks.

### Secondary Efficacy Endpoints:

Change from baseline in frequency of weekly RP attacks.
Change from Baseline in average duration of weekly RP attacks.
Change from Baseline in average severity of weekly RP attacks.
Change from Baseline in average daily RCS.
Change from baseline in highest (most severe) pain score recorded during weekly RP attacks.
Change from baseline in average pain score recorded during weekly RP attacks.
Change from baseline in net digital ulcer burden.
Change from baseline in distal-dorsal difference (DDD) of the affected index finger sites.
Change from baseline in participant quality of life and other aspects of scleroderma disease measured using the Scleroderma Health Assessment Questionnaire (SHAD).
Change from baseline in participant gastrointestinal symptoms (of sclerosis) as assessed with the UCLA SCTC GIT 2.0 questionnaire.
Change from baseline in Raynaud-visual analog scale (VAS).
Change from baseline in physician assessment of disease.
Measurement of cilnidipine drug levels taken 2 to 6 hours following the last dose.
The time to reach maximum degree of efficacy (in days) compared to baseline.
The time to return to baseline symptom severity after termination of dosing.
Impact of daily ambient temperature on symptomatic RP attacks.
Use of rescue medications for breakthrough symptoms.

### Exploratory Efficacy Endpoints:

Change from baseline in endothelial function as measured by reactive hyperemia index (RHI) using Endo-PAT
Change from baseline in novel patient-reported outcomes (ASRAP score) which assesses the severity and impact of RP in SSc (Part B).
Change from baseline in combination endpoint (frequency of weekly RP attacks and ASRAP score) (Part B).
Change from baseline in reported ocular symptoms (Part B)
Effect of combination therapy (Cilnidipine 10 mg+Tadalafil 5 mg, and Cilnidipine 20 mg+Tadalafil 5 mg) on all efficacy endpoints (Part A only).

### Safety Endpoints:

Incidence of treatment emergent adverse events (AEs) and serious adverse events (SAEs), including clinically significant vital signs. For Part A safety will be assessed from the time of randomization until 7 days following the last protocol dose. For Part B, participants will be followed for 28 days from the last study dose.
Serious adverse events will be monitored by a DSMB (Part A) and Drug Safety Officer (Part B) on an ongoing basis throughout the study.
Adverse events will be coded using the MedDRA and summarized by SOC, PT, and treatment group.

### Statistical Methods:

Part A data will be analyzed in exploratory fashion to support cilnidipine dose selection and treatment effect check for sample size confirmation for Part B. For Part B data, mixed effects model will be used for analysis of the continuous efficacy endpoints in the crossover design. For nominal data, Chi-square tests will be applied. Generalized Estimating Equations method will be used, as appropriate, for adjusting for potential confounding factors. Safety endpoints will be summarized by treatment group. No multiple comparison adjustment will be used to control alpha for the multiple comparisons.

### Statistical Analyses:

Data from Part A and Part B will be analyzed separately. The primary and secondary efficacy analyses will be based on the Modified Intent-To-Treat (mITT) population. Analyses based on the Intent-To-Treat (ITT) and PP population for Part B will be considered secondary and confirmatory. All safety analyses will be performed on the safety population. Subgroup analyses will also be performed.

Additional exploratory analyses may be performed and will be documented in the statistical analysis plan. Any deviation from planned analyses described in this protocol will also be documented in the statistical analysis plan. Data will be summarized by treatment group for treatment effect comparison according to crossover design; participants receiving each treatment will be pooled from all periods. Baseline will be defined as screening assessments for change from baseline analyses for all periods.

### Sample Size Calculation:

The sample size for Part B was calculated based on the available data from Part A at the time of protocol specified data review Assuming a 2-sided 0.05 alpha for the comparison of cilnidipine 20 mg versus placebo, between-participants standard deviation (SD) of 35, a correlation between the two measurements on the same participant of 0.2, and a 25% dropout rate, it is estimated that a total sample size of 38 participants (19 in each treatment sequence) is needed to obtain complete data from 28 participants (14 in each treatment sequence), for ≥80% power to detect a decrease of 25 in percentage change from baseline in weekly RP attacks in a 2×2 crossover design.

### Pharmacokinetics and Pharmacodynamics

### Pharmacokinetics

Cilnidipine achieves maximal plasma concentration in about 2 hours, which is more rapid than other CCBs (e.g., amlodipine at 10 hours or extended use nifedipine at 3 hours), which may increase cilnidipine's suitability for treating SSc patients. See Table S3 for a summary of pharmacokinetic (PK) parameters for cilnidipine.

TABLE S3
PK Parameters for 10 mg Cilnidipine,
Healthy Volunteers, single oral dose

| Pharmacokinetic Parameters | Mean |
|---|---|
| Cₘₐₓ (ng/mL) | 8.9 (±4.2) |
| Tₘₐₓ (hr) | 2 (0.5-5) |
| AUCₗₐₛₜ (ng*hr/mL) | 37.0 (±17.9) |
| AUC_{inf} (ng*h/mL) | 41.2 (±20.4) |
| CL/F (L/hr) | 304.7(±147.5) |
| T_{1/2} (hr) | 7.5(±1.0) |

Source: Lee, et al., Drug Des Devel Ther. 2014; 8: 1781-1788. 2014
*Mean (±standard deviation), except for Tₘₐₓ for which median (minimum-maximum) is presented.
Abbreviations: AUC_{inf} = area under the plasma concentration time curve from time zero extrapolated to infinity.
AUCₗₐₛₜ = area under the plasma concentration time curve to the last observation.
Cₘₐₓ = maximum plasma concentration.
CL/F = apparent clearance.
Tₘₐₓ = time to reach Cₘₐₓ.

### Pharmacodynamics

The mechanism of action of cilnidipine offers unique potential benefits for SSc participants that differentiate it from other dihydropyridine CCBs. In hypertensive patients, cilnidipine has been shown to have similar equipotent efficacy when compared to other calcium channel antagonist hypertensive treatments, while exhibiting a better safety profile. This is due to cilnidipine's N-type Ca channel selectivity, in addition to its L-type Ca channel activity.

Currently approved CCBs have primarily L-channel Ca activity and little or no N-type activity. Because of its improved safety profile, cilnidipine can be dosed at higher dose levels than other non-N-selective CCBs, engendering greater efficacy in reducing blood pressure.

Unlike other CCBs, cilnidipine with its primarily N-type Ca channel activity also inhibits sympathetic nervous system activity, dilates venules in addition to arterioles, improves endothelial structure and function, and may provide analgesic effects. Cilnidipine has also demonstrated anti-fibrotic effects in nonclinical studies as well as additional renal and cardiovascular effects in clinical studies. Cilnidipine also is a potent inhibitor of the purinergic P2X7R pathway, and studies have shown that fibroblasts from patients with SSc show upregulation of this receptor and that it promotes a fibrogenic phenotype in their fibroblasts. These additional pharmacodynamic properties of cilnidipine address several key factors of SSc and may provide superior treatment for SSc participants than currently available treatments. This is a first in human (FIH) study of cilnidipine and tadalafil combination. No clinical studies of cilnidipine and tadalafil combination have been conducted to date. However, based on the clinical use of CCBs in combination with PDE5 inhibitors, no drug-drug interaction is expected.

### Safety Criteria for Stopping or Unblinding Treatment

Administration of study drug may be paused, and emergency unblinding of treatment conducted following consultation between the Investigator, the Medical Monitor, and the Sponsor representative under the following circumstances:
Symptoms of possible allergic phenomena: rash, hives, urticaria, changes in breathing or wheezing.
Systolic BP (SBP)<90 mmHg
Reduction in BP (relative to baseline values) considered to be significant in the opinion of the Investigator on an absolute basis or consistent with symptoms (dizziness, light-headedness) suggestive of being related to reduction in BP.
Two separate SBP measurements of <90 mmHg, taken at rest (where baseline SBP exceeded 110 mmHg), even in the absence of symptoms, shall be considered sufficient reason to unblind the study for that patient.
Dizziness, when going from a recumbent to standing position and/or if accompanied by a reduction in BP.
Dizziness in the absence of a reduction in BP should be considered on an individual participant basis, as to its relative degree of severity, as to whether the participant continues in the study.
Unexpected adverse event or reaction.

### Study Termination

The study will be completed as planned unless:
New information or other evaluation regarding the safety of the study medication indicates a change in the known risk/benefit profile for the compound, such that the risk/benefit is no longer acceptable for participants participating in the study. This may be determined by the Sponsor, the Investigator, the HREC or regulatory authorities.
The study is terminated by the Sponsor for administrative reasons.
Participant Population

The study will be conducted in participants aged 18-90 years, diagnosed with severe secondary Raynaud's disease (RCS≥40 and at least a 2-phase color change in fingers of pallor, cyanosis, and/or reactive hyperemia in response to cold exposure or emotion) mostly resulting from SSc (defined by consensus criteria 2013 American College of Rheumatology [ACR]) and exhibiting a frequency of attacks (at least one per day) during the screening period.

Women of childbearing potential will be included and are subject to contraceptive requirements during the study from screening until study completion, including the follow-up period, and for at least 30 days after the last dose of study drug (see Section 4.2). Women of childbearing potential must demonstrate negative pregnancy testing at screening. This is in line with regulatory Guidance on Nonclinical Safety Studies for the Conduct of Human Clinical Trials and Marketing Authorization for Pharmaceuticals (US FDA Guidance document, January 2010).

### Number of Participants

Up to, 76 participants will be enrolled in this study. Thirty-six participants were planned for the parallel-group dose selection phase (Part A), however following the DSMB recommendation to proceed directly to Part B (the powered cross-over phase), Part A was stopped early with 27 participants randomized. In Part B, 38 participants will be randomized in a 2-way crossover design. Participants who complete Part A without any major protocol deviations or compliance issues will be invited to participate in Part B. Following completion of Part A, participants would need to provide written informed consent and meet all eligibility criteria again in order to be randomized into Part B. Sample size assumptions account for a dropout rate of 25%, therefore dropouts will not be replaced.

### Participant Inclusion Criteria

To be eligible for this study, a participant has to meet all of the following inclusion criteria:
1. Male or female participants, aged 18 to 90 years (inclusive at the time of informed consent).
2. Severe secondary Raynaud's disease (defined as RCS at baseline of ≥20) based on ACR criteria mostly resulting from SSc.
3. Regular and frequent Raynaud's attacks (averaging at least one attack per day) during the screening week (in participants with at least a 2-phase color change in fingers of pallor, cyanosis, and/or reactive hyperemia in response to cold exposure or emotion).
4. Willingness to complete the daily diary entry's during the screening period.
5. Participants must be willing and able to provide written informed consent after the nature of the study has been explained and prior to the commencement of any study procedures.
6. Willingness to forego therapies for Raynaud's during study participation is preferred (except for selective serotonin reuptake inhibitors such as fluoxetine which should not be discontinued) Ongoing treatment with CCBs and other routine therapies for SSc-RP is permitted however, if it is not clinically feasible to stop therapy, the participant has been on a stable dose for the last 2 months and meets all eligibility criteria. Rescue therapy (with acetaminophen, nonsteroidal anti-inflammatory drug [NSAIDs], other codeine analgesics, fluoxetine, angiotensin II receptor blockers (ARBs) such as losartan) or CCBs will be allowed to manage breakthrough symptoms of SSc-RP but must be documented).
7. Participants who are on a stable dose (no change in the last 2 months) of a CCB for hypertension or PDES inhibitors for pulmonary hypertension may continue these agents at this stable dose as long as they meet other inclusion criteria during screening.
8. Have agreed not to use (or initiate treatment with) other investigational therapies or unapproved therapies during the course of the study outside of protocol allowances for rescue medication (such medications include but are not limited to: nitroglycerin, topical creams, fenoldopam, nimodipine, amlodipine, fluoxetine, pregabalin, gabapentin, verapamil, sildenafil, tadalafil, vardenafil).
9. Women of childbearing potential (WOCBP) who have agreed to use an effective method of contraception during the study and for 30 days after the last study dose. Acceptable methods of birth control in this study include oral contraceptive, barrier control, or implanted devices. A woman must agree not to donate eggs (ova; oocytes) for purposes of reproduction for at least 30 days after last dose of study drug.
10. Post-menopausal females, aged over 45 years who have not had a period for at least 12 months, and are not using hormonal contraception, or females documented as permanently sterile (e.g., bilateral tubal ligation; hysterectomy, bilateral salpingectomy).
11. Negative urine pregnancy test on randomization day (WOCBP only).
12. All sexually active men whose partner is a WOCBP (due to potential risk of drug exposure through the ejaculate) who agree to a barrier method of birth control during the study and for 30 days after the last dose of the study drug. All men must agree not to donate sperm for at least 30 days after receiving last dose of study drug.
13. Participants must have clinical laboratory values within normal range as specified by the testing laboratory at their most recent pre-screening sample, unless deemed not clinically significant by the Investigator or delegate. Lab sample may be within the last 2 months as long as there have been no significant clinical changes since the last labs and no new medications (e.g. Diuretics) have been introduced that are known to be associated with changes in clinical chemistry or hematology values (e.g. potassium with diuretics or cyclosporine associated with aplastic anemia).

### Participant Exclusion Criteria

A participant who meets any of the following exclusion criteria must be excluded from the study:
1. Primary Raynaud's disease.
2. History of Raynaud's attacks of sufficient severity as to require in-patient hospitalization (within the last 6 months).
3. The SBP of <95 mm Hg during randomization visit (Day 0).
4. Participants with an allergy to dihydropyridine CCBs that results in clinical findings such as profound hypotension, hives, rash, urticaria, wheezing and changes in breathing (Common treatment limiting adverse events [AEs] that occur with CCBs nifedipine and amlodipine such as edema, headache, heart rate changes, tachycardia, fatigue, constipation, flushing, drowsiness, dizziness should not limit enrollment into this study).
5. History of other chronic pain condition that could confound recording of pain scores during the study period.
6. Any prior or ongoing medical conditions, medical history, physical findings, or laboratory abnormality that in the opinion of the Investigator(s), would contraindicate administration of the study medication, or interfere with the study evaluations, or interfere with the participant's ability to comply with the study protocol.
7. Cognitive or language difficulties that would impair completion of the study assessments.
8. Use of any investigational product (IP) or investigational medical device or participation in investigational drug studies within 30 days prior to enrollment in the study.
9. Those receiving nitrates (nitro-dur, nitroglycerin) or similar agents that have vasodilatory effects (e.g., nicorandil) prescribed to treat angina, alpha blockers, PDE inhibitors, prostacyclins or endothelin antagonists for whom dose is not stable for >2 months
10. History of orthostatic hypertension, dizziness or fainting spells, acute coronary or cerebrovascular event within 3 months of study enrollment.
11. History of major thoracic, abdominal, or vascular surgery within 6 months of study enrollment; History of sympathectomy in the hand which is symptomatic for RP.
12. Severe cardiomyopathy, severe valvular heart disease, chronic kidney disease (CKD) stage 3 or greater, evidence of malignancy, end stage lung disease.
13. Pregnant or lactating women.
14. Women of childbearing potential (WOCBP) unable to comply with contraceptive requirements during the study period.
15. Males with partners who are WOCBP and are unable to comply with the contraceptive requirements during the study.
16. History of drug (including recreational use of inhaled amyl nitrates or party poppers) or excess alcohol use that in the opinion of the Investigator(s) would affect the participant's ability to reliably participate in the study. NHMRC guidelines for regular alcohol consumption in healthy adults are no more than 10 standard drinks per week and no more than four standard drinks on any one day.
17. Heavy smokers of tobacco products of any type (defined as >10 cigarettes per day). Light smokers who agree to keep their smoking stable for the duration of their trial participation are eligible.

### Screen Failures

Screen failures are defined as participants who consent to participate in the clinical study but are not subsequently randomized in the study. A minimal set of screen failure information is required to ensure transparent reporting of screen failure participants to meet the Consolidated Standards of Reporting Trials publishing requirements and to respond to queries from regulatory authorities. Minimal information includes screen failure details and eligibility criteria. Participants who withdraw from the study, for any reason, prior to randomization will be considered screen failures. Individuals who do not meet the criteria for participation in this study (screen failure) may be re-screened following a one month waiting period. Re-screened participants should be assigned a new participant number.

### Participant Replacement

All participants who are randomized will be followed and included in the primary ITT analysis. Dropouts will not be replaced.

### Participant Withdrawal Criteria

Participants may withdraw their consent to participate in the study at any time. If a participant withdraws consent, the date and reason for consent withdrawal should be documented. Participants will be encouraged to remain in the clinic to complete all necessary assessments and until the Investigator deems that it is safe to be discharged. Participant data will be included in the analysis up to the date of the withdrawal of consent.

Apart from withdrawal of consent, reasons for early termination of individual participants can include:
Protocol deviations or participant non-compliance (must be specified on the appropriate eCRF)
Serious or severe AEs
Administrative decision by the Investigator or the Sponsor
Death
Other (must be specified).

The primary reason for withdrawal will be identified and recorded on the appropriate eCRF, along with the date of withdrawal.

In accordance with applicable regulations, a participant has the right to withdraw from the study, at any time and for any reason, without prejudice to future medical care.

If a participant is withdrawn because of an AE, the Investigator must arrange for the participant to have appropriate follow-up care until the AE is resolved or has stabilized. Unresolved AEs will be followed until the last scheduled Follow-up/End of Study (EOS) visit or until the Investigator(s) determine that further follow-up is no longer indicated. In addition to AEs, other reasons for removal of participants from the study might include, but are not limited to, withdrawal of consent, administrative decision by the Investigator or the Sponsor, protocol deviation, or participant noncompliance.

If a participant asks or decides to withdraw from the study, all efforts will be made to complete and report the observations, especially those related to the listed primary and secondary objectives, as thoroughly as possible up to the date of withdrawal. Wherever possible, the tests and evaluations, including those listed for the EOS/follow-up visit, should be performed for all participants who discontinue prior to the completion of the study.

### Treatments

### Cilnidipine

Cilnidipine is an orally administered dihydropyridine CCB that dilates blood vessels, increases blood flow, inhibits sympathetic nervous system activity, and improves endothelial structure and function. Please refer to IB for more information on composition of cilnidipine tablet.

Profervia® tablets are white film-coated tablets. Each tablet contains cilnidipine (10 or 20 mg) with microcrystalline cellulose, lactose, magnesium stearate, sodium starch glycollate, Opadry white, polyvinyl alcohol, titanium dioxide, macrogol, talc, and purified water.

Cilnidipine is commercially available and should be used only in accordance with this study protocol and IB. Cilnidipine 10 mg and 20 mg oral tablets will be provided to the site in cartons containing 16 tablets sealed in blister packs.

### Tadalafil

Tadalafil for oral administration belong to a class of medications called PDE5 inhibitors.

Tadalafil is commercially available and should be used only in accordance with this study protocol. Please refer to the pharmacy manual and product information sheet for more information on composition and storage information for tadalafil. Tadalafil will be over encapsulated (and back filled with inert capsule filler consisting only of maize starch and pre-gelatinized maize starch, so that the internal tablet cannot be detected) and provided in bottles containing 16 capsules to the site.

### Reference Products

Placebo tablets (matching cilnidipine) and placebo capsules (matching tadalafil) for oral administration will be provided for this study.

### Dosage and Treatment Periods

In Part A, each participant will take daily one capsule and one tablet to blind the active therapy being received. In Part B, each participant will only take one tablet daily. All medications for each dosing period (each dosing period will last for 12 days [±2 days]) will be dispensed during the preceding in-clinic visit and then self-administered by the participant once daily, orally, in the morning.

If a participant accidentally misses a dose, they should be advised to take the dose on the same day as soon as they realize. Only one dose should be taken each day. If more than one dose is lost, the participant should notify the study staff so that their in-clinic visit can be adjusted if needed.

Part A, Double-blind, Parallel-group, Dose Selection Study drug will be self-administered daily for 12 (±2) days. Each participant will receive only one treatment. Please refer to FIG. 2 for more details.

Part B, double-blind, placebo-controlled, 2-way crossover study drug will be self-administered daily in two dosing periods separated by a four-day (±1) washout period. Each participant will receive a different treatment during each dosing period, with a total of two treatments received. Please refer to FIGS. 3A and 3B for more detail.

### Method of Assigning Participants to Treatment

### Randomization

A randomization list will be prepared using a statistical software package by a Biostatistician. Each participant will be provided with a unique screening number post-documentation of informed consent. Once deemed eligible, the participant will be assigned a sequential randomization number prior to first dosing. Participants who consent to screening but then withdraw from the study or fail eligibility, for any reason, prior to randomization will be considered screen failures.

Participants who complete Part A without any major protocol deviations or compliance issues will be invited to participate in Part B. Those who consent to be rescreened for Part B will be provided with a second unique screening number. If deemed eligible, the participant will be assigned a sequential randomization number specific to Part B.

### Concomitant Medications

All medications, including over the counter medications, vitamins, and herbal supplements, taken during the screening period will be reviewed by the Investigator to determine whether these medications render the participant as suitable for inclusion in the study.

Concomitant medications of interest will be captured electronically from the start of the screening period until study completion.

Treatment prior to enrollment with therapies for SSc-RP including but not limited to CCBs, nitroglycerin, topical creams fenoldopam, nimodipine, fluoxetine, pregabalin, gabapentin, sildenafil, tadalafil, vardenafil are permitted. In order to be eligible, participants must be willing to forego these therapies for SSc-RP at the start of the screening period and for the duration of the study. Participants who are on a stable dose (no change in dose in prior 2 months) of a CCB for hypertension or sildenafil for pulmonary hypertension may continue these agents at this stable dose as long as they meet other inclusion criteria during screening. Similarly, participants who are receiving CCBs to manage their symptoms of SSc-RP and are unwilling to stop treatment for the duration of the study would still be eligible if the participant's dose has been stable for the past 2 months. During the study participants will be able to decrease the dosage of their prior CCB for safety reasons only; no increase in dosage will be allowed during the study period. The use of any other IP or investigational medical device within 30 days prior to screening is prohibited.

Prior therapy or concomitant therapy (after study drug administration) with any medications, including both prescription and non-prescription drugs should be discussed with the Investigator and Sponsor's MM before study drug administration, except in the case of necessary treatment of AEs or where appropriate medical care necessitates that therapy should begin before the Investigator can consult with the Sponsor's MM.

### Rescue Medicine

Medications required as rescue therapy can be taken to manage breakthrough symptoms of SSc-RP but must be recorded in the participant Diary. First-line therapy may include acetaminophen, NSAIDs, or other codeine-based analgesics. These rescue medications may be taken for the duration of symptoms of a Raynaud's attack. For participants in whom first-line rescue therapy is not effective, additional rescue medication therapy may be started per Investigator discretion and could include fluoxetine, ARBs such as losartan or CCBs. Rescue therapy should continue as long as clinically needed during an acute attack, but then patients should return to the pre-rescue study medication regimen. All participants receiving rescue therapy should continue in the study undergoing subsequent dosing periods through study completion.

### Treatment Compliance

All doses will be self-administered by participants remote from study sites (at home). For each dosing period, participants will be dispensed with two weeks' worth of study medication and will be asked to return the unused study medication on the last day of each dosing period at the time of in-clinic visit. The treatment compliance will be noted by the Investigator(s) during the in-clinic visit.

### Blinding

This study is double-blind. To maintain the blind, all study medication will be provided to the site in a blinded fashion. Cilnidipine tablets and matching placebo will be supplied in cartons containing 16 tablets sealed in blister packs, identical in appearance. Tadalafil will be provided in an over encapsulated form. The capsules, tadalafil, and placebo will be identical in appearance and weight and will be supplied in bottles containing 16 capsules. Each study drug will be labeled with a unique ID number. The interactive voice response system (IVRS) will have access to the treatment arm assignment for each individual ID number.

### In the Event of an Emergency: Unblinding a Code

It is recognized that, in the course of clinical practice, it may be necessary for the treating physician to have knowledge of the treatment assignment to ensure the safety of a study participant. This circumstance is extraordinary and will likely impact a minor fraction of the enrolled participants. Unblinding will be done via the IVRS. The treating physician is encouraged to contact the Sponsor MM in this circumstance. The Sponsor and DSMB will monitor all episodes of unblinding very carefully.

### Study Schedule

The SoAs for Part A and Part B of the study are provided in FIG. 2 and FIGS. 3A and 3B. Where possible, assessments should be conducted in order of least invasive to most invasive. This study consists of four periods:
Screening period (begins with initial participant contact through participant completion of the screening Diary)
Randomization period (from the time participant eligibility is confirmed and the participant randomized until immediately before the 1^{st} dose of study drug)
Procedural period (from the first dose of study drug in the first dosing period until the last day of dosing)
Follow-up period (from the end of the procedural period through completion of follow-up for safety i.e., 7 days for Part A and 28 days for Part B).

### Part A—Double-Blind, Placebo-Controlled, Parallel-Group, Dose Selection

In Part A, the procedural period will require only one dosing period i.e. participants will receive only one treatment during the procedural period. Within the procedural period for Part A there will be two sub-periods:
Daily at home dosing (first ten to fourteen days of the dosing period)
In-clinic visit (the last day of the dosing period that occurs on Day 12 [±2 days]).
   Part B—Double-blind, Placebo-controlled, 2-way Crossover

In Part B, the procedural period will require two dosing periods i.e., participants will receive two different treatments in a 2-way crossover design. Within the procedural period for Part B there will be two sub-periods associated with each dosing period/treatment received:
Daily at home dosing (first ten to fourteen days of the dosing period)
In-clinic visit (the last day of the dosing period that occurs on Day 12 [±2 days]) One washout period (four days of no dosing) is required between each dosing period).

### Screening (Day −10 to Day −1)

Prior to enrolling in the study, and before performance of any procedures, potential participants will be contacted via phone to discuss the details of the study and assess their eligibility and willingness to comply with all study procedures and duration. A copy of the Informed Consent Form (ICF) will be emailed to the patient in conjunction with this discussion. If the participant seems eligible and is interested in participating in the screening period, then they will be asked to start using a diary to record the daily clinical features and symptoms of their SSc-RP for the next 7 to 10 days. Commencement of diary use will be considered implied consent for the screening period, the data from which will be used to confirm eligibility and future baseline analyses assuming the participant is randomized.

During screening, the Diary will collect the following data to confirm eligibility and serve as the baseline measure for efficacy endpoints should the participant be randomized:
Number of daily Raynaud's attacks
Duration of each attack
Symptoms of each attack, including numbness, pain, tingling, color changes
Severity of each attack (all symptoms of the attack to be considered including tingling, numbness, pain, color changes)
Location of participant during each attack (inside/outside)
Pain score of each attack-using 11-point Likert scale, a validated pain scale which can be used to record intensity of pain.
Daily RCS— a validated outcome measure used to assess the level of difficulty experienced due to RP each day.

Rescue medications taken to manage breakthrough symptoms of SSc-RP (including acetaminophen, NSAIDs, other codeine-based medicines, fluoxetine, ARBs such as losartan, CCBs) as well as other concomitant medications will also be captured and assessed during screening.

### Randomization Day (Day 0)

Participants will be scheduled to visit the clinic for randomization (Day 0) assessments between days 7 to 10 of the screening period. Only participants who seem eligible based on Diary compliance and frequency of RP attacks will be requested to visit clinic for randomization. During the visit the participant will be provided with another copy of the ICF. Prior to being asked to sign the consent form, participants will be given time to review study information and ask any questions.

After the consent form is signed and the following assessments will be carried out:
Medical history/demographics
Previous/concomitant medications
Vital signs
Physician's Assessment of Disease (including RP, scleroderma, ulcers, ocular symptoms, overall health)
Pregnancy test
Review of daily participant dairy
SHAQ which includes Raynaud's VAS to be completed by the participant with review by physician designee
Assessment of gastrointestinal symptoms (of sclerosis) using the UCLA SCTC GIT 2.0 questionnaire, to be completed by the participant with review by physician designee
Assessment of Systemic sclerosis-associated Raynaud's Phenomenon (ASRAP) questionnaire to be completed by the participant with review by physician designee (Part B)
Digital ulcer assessment
Thermography
Endo-PAT
Blood sample for PK
Inclusion/Exclusion
Randomization
AE/SAE reporting
Dispensing study medication
   Note: The data collected for assessments that are performed first time on randomization (Day 0) visit (vital signs, digital ulcer assessment, and Endo-PAT) will serve as baseline measure for efficacy endpoints for those assessments.

Routine hospital tests including hematology, biochemistry, inflammatory markers (C-reactive protein [CRP] and erythrocyte sedimentation rate [ESR]), antibody status (serum anti-topoisomerase [anti-Scl 70]) and nailfold capillaroscopy should be conducted as clinically indicated per standard of care but are not required per protocol. If conducted, results will be collected in the eCRF and used to describe the severity of disease in the baseline demographic and disease data.

### Dosing Periods

### Daily at Home Dosing (12 Days (±2 Days))

During the dosing period, participants will be required to self-administer the assigned study medication once daily in the morning at home. Participants will also be required to complete their Diary daily to capture the clinical features and symptoms of their SSc-RP, and report concomitant medications including rescue therapy (if any).

### In-Clinic Visit (Last Day of the Dosing Period)

Participants will be required to visit the clinic following each dosing period—dosing day 10 to Day 14. The day of the in-clinic visit is considered the last day of dosing in each dosing period. After taking their last dose of study medication in the morning at home, the following assessments/procedures will take place during the in-clinic visit by the physician or designee, with results recorded in the eCRF:
Vital signs
Blood sample for PK
Daily participant E-dairy review for the most recent dosing sequence
Scleroderma Health Assessment Questionnaire (SHAD) which includes Raynaud's VAS to be completed by the participant with review by physician designee
Assessment of gastrointestinal symptoms (of sclerosis) using the UCLA SCTC GIT 2.0 questionnaire, to be completed by the participant with review by physician designee
Assessment of Systemic sclerosis-associated Raynaud's Phenomenon (ASRAP) questionnaire, to be completed by the participant with review by physician designee (Part B)
Physician's Assessment of Disease (including RP, scleroderma, ulcers, ocular symptoms, overall health)
Concomitant medications
Thermography (for two most symptomatic areas in terms of Raynaud's)
Endothelial dysfunction (Endo-PAT)
AE/SAE reporting
Dispensing/returning study medication
   Note: On the last clinic visit at the end of last dosing period (dosing period 1 for Part A and dosing period 2 for Part B), pregnancy test will be performed for WOCBP.

### Washout Period

In Part B, each dosing period will be separated by a 4-day (±1 day) washout period. The washout period will commence the day after the in-clinic visit during which participants will not take any study medication. During the washout period, participants will be required to complete the daily participant Diary, reporting their symptoms of SSc-RP, and use of any concomitant medications. Once the 4-day washout is completed the participant will commence the daily at home dosing for the study medication dispensed at the previous in-clinic visit. No washout period is required after the second and final in-clinic visit. After this visit participants will proceed directly to follow-up.

Follow-up

All Participants will be followed for 7 days following completion of the final dosing period for their symptoms of SSc-RP, during which time they will be requested to continue to complete their patient Diary. Participants will also be requested to report use of any concomitant medications and any AEs/SAEs during the Follow-up period. Participants in Part B will be followed for AEs/SAEs until 28 days following the last dose of study medication. Follow-up of AEs/SAEs will cease at 7 days for participants in Part A. This visit marks the end of participation in this study.

### Early Termination (If Applicable)

Participants who withdraw early from the study will be encouraged to return to the clinic for an EOS assessment. The following procedures will be conducted:
Participants will be requested to complete the Diary for 7 days following termination
Participants will be requested to report use of concomitant medications of interest for 7 days following termination
Participants will be requested to report any AEs/SAEs for 7 (Part A) to 28 (Part B) days following termination. This visit marks the end of participation for participants that withdraw early from the study.

### Pharmacokinetic Assessments

Blood Sample Collection

When possible, one 4 mL blood sample will be obtained during each in-clinic visit within 2 to 6 hours of last dose of study drug in that dosing period as delineated in the SoA (FIG. 2 and FIGS. 3A and 3B). The level of cilnidipine in blood will be measured following last dose of the dosing period. Sample handling details will be provided in the PK manual. The actual collection time of each sample must be recorded in the source data documentation, on the collection tube and in the eCRF.

### Efficacy Assessments

### Efficacy Parameters

Study procedures should be completed as delineated in the SoAs (FIG. 2 and FIGS. 3A and 3B).

### Diary

The Sponsor-developed participant-informed Diary will be used in this study to record data. Participant will be required to keep and fill the Diary daily as delineated in the SoAs (FIG. 2 and FIGS. 3A and 3B).

The relevant metrics measured by this tool daily are:
Study medication
SSc-RP symptoms (Reporting an attack including duration of attack)
Severity of the attack considering all symptoms of the attack e.g. tingling, numbness, pain, color changes (VAS 0-10 cm scale)
Participant's location at the time of the attack (inside home/outside home)
Selecting symptoms experienced during the attack (tingling, numbness, pain, color changes, other)
Pain rating during the attack (11-point Likert scale)
The RCS based on how much difficulty participants had with Raynaud's today, how many attacks the participant had, and how long they lasted. Participants will also be asked to consider how much pain, numbness, or other symptoms the Raynaud's caused in fingers (including painful sores), and how much the Raynaud's alone affected the use of hands today (VAS 0-10 cm scale).

### Metrics Measured at the In-Clinic Visit (Once in a Dosing Period) are:

Digital ulcer severity (VAS 0-10 cm scale)— Applicable only if participant has digital ulcers.
Scleroderma Health Assessment Questionnaire which includes Raynaud's VAS measuring participant quality of life over the past 7 days
UCLA SCTC GIT 2.0 questionnaire, assessing gastrointestinal symptoms (of sclerosis) over the past 7 days.
Assessment of Systemic sclerosis-associated Raynaud's Phenomenon (ASRAP) questionnaire, measuring participant experience with Raynaud's symptoms over the past 7 days.
   Note: Digital ulcer severity (VAS 0-10 cm scale), SHAQ, UCLA SCTC GIT 2.0 and ASRAP questionnaire will be done on paper in-clinic. external temperature will be a feed into the Diary database based on participant's location.

Physician Assessment of Disease

The Physician will assess the below at each in-clinic visit, details of which will be recorded in the eCRF.
Rating severity of participant's Raynaud's disease
Rating severity of participant's scleroderma symptoms
Rating severity of participant's ulcers how many ulcers, how many considered new, for each ulcer: location and diameter of the ulcer
Rating overall health of participant for past week
Any ocular symptoms, and the nature of these symptoms as ocular manifestations are common in patients with SSc (Kozikowska 2020), and range in incidence and prevalence from over 80% having symptoms of dry eye disease, and >75% having eyelid symptoms and conjunctival symptoms in >70% of patients.
Patient symptoms will be recorded in severity with the patient asked to rate any ocular symptoms or discomfort or pain on a 0-10 scale and the physician will record these answers in the eCRF. AE/SAE reporting.

Drug accountability, including dispensing and returning of the study medication will also be recorded at each visit.

### Scleroderma Health Assessment Questionnaire (SHAQ)

The standard, validated, patient reported outcome measures tool for SSc patients, the SHAQ, will be collected at the time points specified in the study schedules (FIG. 2 and FIGS. 3A and 3B) to assess the participant's quality of life. The SHAQ includes a Raynaud's VAS that will also be reported separately.

### UCLA Scleroderma Clinical Trials Consortium Gastrointestinal Tract 2.0 (UCLA SCTC GIT 2.0)

The standard, validated, patient reported outcome measures tool for SSc patients, the UCLA SCTC GIT 2.0 will be collected at the time points specified in the study schedules (FIG. 2 and FIGS. 3A and 3B) to assess gastrointestinal symptoms (of sclerosis).

Assessment of Systemic Sclerosis-associated Raynaud's Phenomenon (ASRAP)

A novel patient-reported outcome (PRO) questionnaire will be completed by the participant at the time points specified in the Part B study schedule FIGS. 3A and 3B), to assess the severity and impact on daily life of RP in SSc.

### Thermography

Thermography assessments will be performed at the time points specified in the study schedules (FIG. 2 and FIGS. 3A and 3B). Thermography will be conducted on the most severely impacted digits identified at screening; the participant must be indoors for at least 30 minutes prior to the test to give the body time to equilibrate. Photos will be taken with the help of Fluor thermographic camera of these two areas at times specified in SoAs.

### Endothelial Dysfunction (Endo-PAT)

Assessments for endothelial dysfunction will be performed using Endo-PAT at timepoints specified in the study schedules (FIG. 2 and FIGS. 3A and 3B). The Endo-PAT is a diagnostic device used to assess endothelial vasodilator function in a rapid and non-invasive fashion. The below points should be considered before assessment is started:
1. Prior to the study, the participant should fast for at least 4 hours, and should refrain from caffeine, vitamins or medications that might affect vascular tone for at least 8 hours. The participant must reconfirm their abstinence from tobacco and may use the restroom prior to the assessment.
2. The Endo-PAT assessment should be conducted in a quiet, dimly lit, temperature-controlled (25° C. for at least 30 minutes) exam room to reduce fluctuations in vascular tone.
3. Cell phones or paging devices should be silenced, and restrictive clothing that could interfere with blood flow to the arms should be removed. The participant should also remove watches, rings, or other jewelry on the hands or fingers.
4. Participant's fingers should be inspected for any deformities or injuries that could affect the study. The probes should not be placed on a finger that is cut or injured. Fingernails should not extend more than 5 mm or ⅕ of an inch beyond the tip of the finger tissue.
5. The index finger is recommended for the study; however, if this finger is unsuitable, a different digit (except the thumb) may be used, as long as the same finger is used on both hands.
6. The participant should be supine and comfortable for 15 minutes so as to attain a cardiovascular steady state.

### Safety Assessments

### Safety Parameters

Study procedures should be completed as delineated in the SoA (FIG. 2 and FIGS. 3A and 3B). Any unscheduled procedures required for urgent evaluation of safety concerns must take precedence over all routine scheduled procedures.

### Demographic/Medical History

Medical history (including alcohol and smoking status), date of birth, age (calculated), weight, sex, ethnicity, and race will be recorded at randomization (Day 0) visit.

### Vital Signs

Vital signs (SBP, DBP, pulse rate, temperature) will be measured at the time points specified in the SoA (FIG. 2 and FIGS. 3A and 3B) with participants resting for at least 5 minutes in a supine position. When the time of vital signs measurement coincides with a blood draw, the vital signs will be taken before the scheduled blood draw where possible, ensuring the blood draw is within the window specified in the protocol.

Additional vital signs may be performed at other times if deemed necessary.

### Laboratory Assessments

Routine hospital laboratory tests including hematology, biochemistry, inflammatory markers (CRP and ESR), and antibody status (Scl-70) should be conducted as clinically indicated per standard of care but are not required per protocol. If conducted, results will be collected in the eCRF and used to describe the severity of disease in the baseline demographic and disease data. Additional clinical laboratory tests may be performed at other times if deemed necessary, based on the participant's clinical condition.

### Pregnancy Testing

A urine pregnancy test will be performed at the randomization (Day 0) visit and on the last clinic visit at the end of last dosing period for WOCBP only.

### Adverse and Serious Adverse Events

In this study, AEs and SAEs will be reported for all participants from the time of randomization until the completion of the Follow-up/EOS visit. Adverse events that are ongoing at the EOS visit will be marked as Not Recovered/Not resolved on the AE eCRF page (see Section 9.4.4). The Investigator will do full AE review during in-clinic visit. All spontaneously volunteered and enquired for, as well as observed AEs, will be recorded in the participant's medical records and the eCRF.

### Complications of the Disease Under Study

Clinical features and symptoms of SSc-RP must be recorded as endpoints in the electronic data collection tools provided by the Sponsor, as well as in the source documents and should not be reported as AEs.

### Definition of Adverse Events

An AE is any event, side effect, or other untoward medical occurrence that occurs in conjunction with the use of a medicinal product in humans, whether or not considered to have a causal relationship to this treatment. An AE can, therefore, be any unfavorable and unintended sign (that could include a clinically significant abnormal laboratory finding), symptom, or disease temporally associated with the use of a medicinal product, whether or not considered related to the medicinal product.

### Events Meeting the Definition of an AE Include:

Exacerbation of a chronic or intermittent pre-existing condition including either an increase in frequency and/or intensity of the condition
New conditions detected or diagnosed after study drug administration that occur during the reporting periods, even though it may have been present prior to the start of the study
Signs, symptoms, or the clinical sequelae of a suspected interaction
Signs, symptoms, or the clinical sequelae of a suspected overdose of either study drug or concomitant medications (overdose per se will not be reported as an AE/SAE).
   Events that do not Meet the Definition of an AE Include:
Medical or surgical procedure (e.g., endoscopy, appendectomy); the condition that leads to the procedure should be reported as an AE if it meets the criteria of an AE
Situations where an untoward medical occurrence did not occur (e.g., social and/or convenience admission to a hospital)
Anticipated day-to-day fluctuations of pre-existing disease(s) or condition(s) present or detected at the start of the study that do not worsen.

If there is evidence of an AE through report or observation, the Investigator or designee will evaluate further and record the following information:
Time of onset and resolution
Severity
Seriousness
Causality/relation to study treatment
Action taken regarding study drug
Action taken regarding AE
Outcome.

### Severity of an Adverse Event

Severity of AEs will be graded by the Investigator as one of:
Mild (Grade 1): A type of AE that is usually transient and may require only minimal treatment or therapeutic intervention. The event does not generally interfere with usual activities of daily living.
Moderate (Grade 2): A type of AE that is usually alleviated with additional specific therapeutic intervention. The event interferes with usual activities of daily living, causing discomfort but poses no significant or permanent risk of harm to the research participant.
Severe (Grade 3): A type of AE that interrupts usual activities of daily living, or significantly affects clinical status, or may require intensive therapeutic intervention.
Life-threatening (Grade 4): A type of AE that places the participant at immediate risk of death.
Death (Grade 5): Events that result in death.

### Causal Relationship of an Adverse Event

The Investigator will assess the relationship between study drug and the occurrence of each AE. The Investigator's assessment of the relationship of each AE to study drug will be recorded in the source documents and the eCRF. Alternative causes, such as medical history, concomitant therapy, other risk factors, and the temporal relationship of the event to the study drug should be considered and investigated, if appropriate. The following definitions are general guidelines to help assign grade of attribution:
Not related: The event is clearly related to other factors such as the participant's environment or clinical state, therapeutic interventions or concomitant drugs administered to the participant. This is especially so when an event occurs prior to the commencement of treatment with the study drug.
Unlikely: The temporal association, participant history, and/or circumstances are such that the study drug is not likely to have had an association with the observed event. Other conditions, including concurrent illness, progression, or expression of the disease state, or reaction to a concomitant drug administered appear to explain the event.
Possible: The event follows a reasonable temporal sequence from the time of study drug administration or follows a known response to the study drug but could have been produced by other factors such as the participant's clinical state, other therapeutic interventions, or concomitant drugs administered to the participant.
Probable: The event follows a reasonable temporal sequence from the time of study drug administration and follows a known response to the study drug and cannot be reasonably explained by other factors such as the participant's clinical state, other therapeutic interventions, or concomitant drugs administered to the participant.
Definite: The event follows a reasonable temporal sequence from the time of study drug administration or control abates upon discontinuation or cannot be explained by known characteristics of the participant's clinical state.
   Action Taken with Investigational Products

Should the Investigator need to alter the administration of the study drug from the procedure described in the protocol due to the wellbeing and safety of the participant then the action taken will be recorded on the AE eCRF page, as one of the following options:
Dose Reduced
Drug Interrupted
Drug Withdrawn
Not Applicable
Other

### Outcome

Outcome of an AE will be recorded on the AE eCRF as follows:
Recovered/Resolved
Recovering/Resolving
Recovered/Resolved with Sequelae
Not Recovered/Not Resolving
Fatal
Unknown

### Definition of Serious Adverse Event

An SAE is an AE occurring during any study phase (i.e. baseline, treatment, washout, or follow-up), and at any dose of the study drug (active or placebo), that fulfills one or more of the following:
Results in death
It is immediately life-threatening
It requires in participant hospitalization or prolongation of existing hospitalization
It results in persistent or significant disability or incapacity
Results in a congenital abnormality or birth defect.
It is an important medical event that may jeopardize the participant or may require medical intervention to prevent one of the outcomes listed above.

Important medical events that may not be one of the above may be considered an SAE by the Investigator when, based upon appropriate medical judgment, they are considered clinically significant and may jeopardize the participant, or may require medical or surgical intervention to prevent one of the outcomes listed above.

An AE is considered “life-threatening” if, in the opinion of either the Investigator or the Sponsor, its occurrence places the participant at immediate risk of death. It does not include an AE that, had it occurred in a more severe form, might have caused death.

### Notification of a Serious Adverse Event

All SAEs, regardless of relationship to the study drug, during the period starting from the time of randomization through to the EOS will be recorded in the eCRF. The Investigator will do full SAE review during in-clinic visit. All SAEs will be recorded in the participant's medical records and the eCRF.

Once the Investigator becomes aware of an SAE, they must report the SAE to Sponsor within 24 hours of knowledge of the event.

If requested, supporting de-identified source documents (e.g., hospital discharge summary, autopsy report when available, results of relevant diagnostic tests completed to evaluate the event) will also be sent to the Sponsor.

A written SAE report must include a full description of the event including the below parameters:
Diagnosis or description of event
Onset date
Severity assessment
Causal relationship to the IP
Assessment of seriousness of the event
Corrective treatment administered for the SAE
Action taken related to study drug include the following: dose interruption, dose delay, dose reduction or study drug discontinuation
Outcome of event and end date.

The Sponsor is responsible for notifying the relevant regulatory authorities of certain events. It is the Investigator's responsibility to notify the HREC of all SAEs in accordance with the HREC SAE reporting policy. The Investigator will also be notified of all unexpected, serious, drug-related events that occur during the clinical study. The investigational site is responsible for notifying its HREC of these additional SAES.

Note: Disease progression will not be considered a reportable event.

### Clinical Laboratory Abnormalities and Other Abnormal Assessments as Adverse Events and Serious Adverse Events

Abnormal laboratory findings or other abnormal assessments (e.g. vital signs) per se are not reported as AEs. However, those abnormal findings that are deemed clinically significant by the Investigator(s) and/or delegate or are associated with signs and/or symptoms must be recorded as AEs if they meet the definition of an AE (and recorded as an SAE if they meet the criteria of being serious) as previously described. Clinically significant abnormal laboratory or other abnormal findings that are detected after randomization or that are present at baseline and worsen after randomization are included as AEs (and SAES if serious).

The Investigator(s) should exercise medical and scientific judgment in deciding whether an abnormal laboratory finding, or other abnormal assessment is clinically significant. To be considered clinically significant, the abnormality should be associated with a clinically evident sign or symptom or be likely to result in an evident sign or symptom in the near term. A clinically significant laboratory abnormality in the absence of clinical symptoms may jeopardize the participant and may require intervention to prevent immediate consequences. For example, a markedly low serum glucose concentration may not be accompanied by coma or convulsions yet be of a magnitude to require glucose administration to prevent such sequelae.

### Recording Adverse Events

Adverse events spontaneously reported by the participant and/or in response to an open question from the study personnel or revealed by observation will be recorded in accordance with the Investigator's normal clinical practice and on the AE page of the eCRF during the study at the investigational site.

However, abnormal values that constitute an SAE or lead to discontinuation of administration of study drug must be reported and recorded as an AE. The AE term should be reported in standard medical terminology when possible. For each AE, the Investigator will evaluate and report the onset (date and time), resolution (date and time), intensity, causality, action taken, serious outcome (if applicable), and whether or not it caused the participant to discontinue the study. AEs that occur during the study must be documented in the participant's medical record, on the AE eCRF and on the SAE report form. If an SAE report is completed, pertinent laboratory data should be recorded on the SAE form, preferably with baseline values and copies of laboratory reports.

In addition, if the abnormal assessment meets the criteria for being serious, the SAE form must also be completed. A diagnosis, if known, or clinical signs or symptoms if the diagnosis is unknown, rather than the clinically significant laboratory finding or abnormal assessment, should be used to complete the AE/SAE page. If no diagnosis is known and clinical signs or symptoms are not present, then the abnormal finding should be recorded.

### Follow-up of Adverse Events and Serious Adverse Events

All AEs and SAEs will be followed for the duration of the study. The Investigator is responsible for ensuring that follow-up includes any supplemental investigations as may be indicated to elucidate as completely as practical the nature and/or causality of the AE/SAE. This may include additional laboratory tests or investigations or consultation with other health care professionals.

The Sponsor may request that the Investigator perform or arrange for the conduct of supplemental measurements and/or evaluations. If a participant dies during participation in the study or during a recognized Follow-up period, the Sponsor should be provided with a copy of any post-mortem findings, including histopathology.

### Pregnancy

Pregnancy testing should be performed in all WOCBP as per the SoA and the pregnancy results should be captured in the eCRF. All WOCBP will be instructed to contact the Investigator immediately if they suspect they might be pregnant (e.g., missed or late menstrual period) at any time during the study. Male participants will contact the Investigator immediately if they suspect they may have fathered a child during the study treatment period. When possible, the partner's pregnancies should be followed (to term) to determine the outcome.

If a participant becomes pregnant during the clinical study, the Investigator will report the details on a pregnancy form to the Sponsor/assigned designee within 24 hours of knowledge of the pregnancy. Even though participants agree to withdraw or terminate the clinical trial, the Investigator should follow-up and document the process and results of all the pregnancies.

If a male participant's female partner becomes pregnant while enrolled in the study, a pregnancy form should be completed. Abortions (spontaneous, accidental, or therapeutic) must also be reported to the Sponsor. Congenital anomalies/birth defects always meet SAE criteria, and should therefore, be expeditiously reported as an SAE, using the previously described process for SAE reporting. A pregnancy form should also have been previously completed and will need to be updated to reflect the outcome of the pregnancy. The Investigator must report any pregnancy (including pregnancy of a male participant's partner), even if no AE has occurred, on a Pregnancy Report Form within 24 hours of the Investigator becoming aware of the pregnancy.

### Study Drug Materials and Management

### Study Drug Packaging and Labeling

The Sponsor is responsible for the preparation and labeling and providing details of batch numbers, safety, and stability data. The study drug will be labeled in accordance with local regulatory requirements and will be shipped at a temperature below 25° C. within a dry place.

### Study Drug Storage

Upon receipt, the study drug must be stored at controlled room temperature (15° C. to 25° C.) in a tightly closed container. The drug should be protected from excess heat and light and should be kept out of reach of children. The Investigator or designee will be fully responsible for the security, accessibility, and storage of the study drug while it is at the investigational facility.

### Administration

The Investigator or designee is responsible for the education of study staff and participants as to the correct administration of the study drug.

### Study Drug Accountability

A record will be maintained by the investigational site that will account for all dispensing and return of any used and unused study drug. At the end of the study, the study drug will be reconciled, and a copy of the record given to the study monitor.

### Study Drug Handling and Disposal

On completion of the study, any surplus study drug supplies will be destroyed upon receipt of written approval from the Sponsor. Evidence of the destruction of any surplus study drug will be supplied to the study monitor. If no supplies remain, this will be documented in the dispensing record.

### Statistics

Statistical methods will be further outlined in the statistical analysis plan (SAP) and approved by the Sponsor. Procedures outlined in the SAP will supersede protocol specified statistical methods in the event of divergence.

Part A and Part B data will be analyzed separately. Analysis of Part A data will be mainly exploratory to support cilnidipine dose selection and treatment effect check for sample size confirmation for Part B. For Part B, the primary and secondary efficacy analyses will be based on the mITT population. Analyses based on the ITT and PP population will be considered secondary and confirmatory. All safety analyses will be performed on the safety population. In general, descriptive statistics (e.g. arithmetic mean, SD, median, minimum and maximum) will be calculated for continuous safety data by treatment and protocol specified time point, while frequency summary (e.g. number of observed and percentage of each categories) will be applied for categorical safety data by treatment and protocol specified time point.

### Sample Size

Assuming a 2-sided 0.05 alpha for treatment (cilnidipine or combination therapy or tadalafil) versus placebo and without controlling alpha for the multiple efficacy comparisons, a sample size of eight participants in each paired comparison group (cilnidipine or combination therapy or tadalafil) is needed for 80% power in a 4×4 crossover design to detect a 25% difference at common SD of 0.5 (a moderate effect size) in percent change from baseline of Raynaud's attack per week. Assuming a 20% dropout rate for final efficacy analysis, ten participants in each group is planned. After reviewing the results from Part A: Run-in phase, the sample size for Part B may be adjusted.

Following from the decision of the committee to exclude tadalafil from Part B of the study, the design was changed to a two-treatments, two-periods (2×2) crossover.

The sample size for Part B was calculated based on the available data from Part A at the time of the protocol specified data review. Assuming a 2-sided 0.05 alpha for the comparison of cilnidipine 20 mg versus placebo, a between-participants standard deviation (SD) of 35, a correlation between the two measurements on the same participant of 0.2, and a 25% dropout rate, it is estimated that a total sample size of 38 participants (19 in each treatment sequence) is needed, in order to obtain complete data from 28 participants (14 in each treatment sequence), for ≥80% power to detect a decrease of 25 in percentage change from baseline in weekly RP attacks in a 2×2 crossover design.

### Analysis Populations

Participant inclusion into each population will be determined prior to the final analysis.

### Intent-To-Treat (ITT) Population

All participants who are entered into the study and complete screening, sign an informed consent for the study and randomized will be included in the ITT population.

Modified Intent-To-Treat (mITT) Population

All participants who are entered into the study and complete screening, sign an informed consent for the study and randomized and have post-baseline attack data (primary endpoint data) will be included in the mITT population.

### Per Protocol Population

All participants who complete the study with all dosing periods (for Part B—at least 5 days of dosing within the last 7 days treatment) and meet all eligibility criteria, and without any major/important protocol deviations, will be included in the PP Analysis Population.

### Pharmacokinetic Population

All participants who receive any amount of active study drug and have sufficiently evaluable concentration time profile to allow determination of at least one PK parameter will be included in the PK population. An evaluable PK profile will be determined at the discretion of the pharmacokineticist following examination of participants with dosing or protocol deviations that could potentially affect the PK profile. The PK population will be used for the summaries of all PK data.

### Safety Population

All randomized participants who received study drug will be included in Safety population and will be classified according to the actual treatment received.

### Statistical Methods

### Participant Disposition

Participant disposition will be analyzed using counts and percentages. The number and percentage of screened participants, enrolled participants, treated participants, participants discontinued from the study and study treatment, as well as the primary reason for discontinuation will be analyzed and listed.

### Demographics, Medical History, and Baseline Characteristics

Demography and baseline characteristics data will be summarized using descriptive statistics. The following demographic variables will be summarized by dose level: race, gender, age, height and weight, concomitant diseases (Hypertension, peripheral vascular disease, diabetes, CKD and stage, osteoporosis, history and type of heart arrhythmia).

In addition, the following baseline characteristics of Raynauds Disease will be summarized: age of onset, seasonality (months disease is worst), usual number of attacks/day, usual peak severity, baseline RCS assessment, how attacks are usually treated, how long attacks last in general, experience with other treatments both pharmacological and non-pharmacological.

### Prior and Concomitant Medication

Prior and concomitant medications will be coded using the most current version of the WHO drug dictionary available at the start of the study. Prior and concomitant medications will be listed by participant and summarized by treatment using anatomical therapeutic chemical (ATC) and preferred name.

### Treatment Compliance and Exposure

Treatment compliance and exposure will be summarized and listed by treatment for all participants in the Safety population.

### Analysis of Primary Endpoint

Percent change from baseline evaluation for frequency of weekly RP attacks will be the primary efficacy endpoint. Data collected in the last 7 days of each dosing period will be used for this analysis. Screening assessments will be used as baseline for the analysis of all periods. No multiple comparison adjustment will be used to control alpha for the multiple comparisons. Mixed effects model will be used for analysis of the primary endpoint according to the crossover design. Other efficacy endpoints of continuous variables will be analyzed using similar methodology. For nominal data, chi-square tests will be applied. Generalized Estimating Equations method will be used, as appropriate, for adjusting for potential confounding factors. In addition, the final analysis will assess whether in this study of severe Raynaud's disease participants, the minimally important difference, previously concluded of 14-15 points on the 100 point RCS scale has been achieved in the cilnidipine dose group. It also will record the percentage of participants achieving a PASS (34 point difference from baseline on a 0-100 VAS) in each treatment group.

### Analysis of Secondary Endpoints

The secondary endpoints of change from baseline evaluation will be compared among treatment groups using mixed effects model. Kaplan-Meier method will be used to evaluate time to event endpoints. To evaluate the impact of daily ambient temperature on symptomatic Raynaud's attack, logistic regression will be used for temperature versus the occurrence of Raynaud's attack (Yes/No).

The effect of temperature on the severity score of Raynaud's attacks and difference of using rescue medication between treatment groups will be evaluated by Chi-square test. The impact of therapy in sympathetic activity will be assessed by mixed model for repeated measures.

### Safety Analyses

All safety assessments, including AEs, laboratory evaluations, vital signs, and other safety assessments will be analyzed using the Safety population.

### Adverse Event

Adverse events will be coded using the most current version of the MedDRA® Version 22.0 or higher. The analysis of AEs will be based on the concept of treatment emergent AEs. Treatment emergent AEs will be tabulated by treatment group and will include the number of participants for whom the event occurred, the severity, and relationship to study drug. Treatment emergent AEs (TEAEs) leading to discontinuation and SAEs with onset after the start of study drug will also be summarized. All AEs and SAEs (including those with onset or worsening before the start of study drug) through the end of the study will be listed.

### Laboratory Evaluations

Baseline laboratory evaluations will be listed and summarized by treatment.

### Vital Signs

Vital signs (BP [systolic and diastolic], pulse rate, and oral temperature) will be listed and summarized by treatment and protocol specified collection time point. Observed and change from baseline will be summarized at each protocol specified collection time point.

### Other Safety Assessments

The following assessments will be listed by participant:
Pregnancy Test
Raynaud's function assessment by physician.

### Pharmacokinetics

Plasma concentrations and actual blood sampling times will be listed by treatment and protocol specified time point and summarized using descriptive statistics—number of measurements, arithmetic mean, SD, and % CV, geometric mean, minimum, median, and maximum—at each scheduled time point. Individual and mean plasma concentration-time profiles will also be presented graphically for each treatment. Pharmacokinetic parameters will be computed from the individual plasma concentrations using a non-compartmental approach.

Value for elimination rate constant (kel), elimination half-life (t½), Area under the plasma concentration-time curve from time zero to infinity (AUCO-inf), apparent total clearance of the drug from plasma after oral administration (CL/F) or apparent volume of distribution during terminal phase after non-intravenous administration (Vz/F) will not be reported. Additional analyses will be performed as deemed necessary upon review of the data.

### Ethics

### Ethics Review

The final study protocol, including the final version of the ICF, must be approved or given a favorable opinion in writing by an HREC as appropriate. The Investigator must submit written approval to the Sponsor before they can enroll any participant into the study.

The Principal Investigator (PI) is responsible for informing the HREC of any amendment to the protocol in accordance with local requirements. In addition, the HREC must approve all advertising used to recruit participants for the study. The protocol must be re-approved by the HREC upon receipt of amendments and annually, as local regulations require.

The PI is also responsible for providing the HREC with reports of any reportable serious adverse drug reactions from any other study conducted with the study drug (active). The Sponsor will provide this information to the PI. Progress reports and notifications of serious adverse drug reactions will be provided to the HREC according to local regulations and guidelines.

### Ethical Conduct of the Study

The study will be performed in accordance with ethical principles that have their origin in the Declaration of Helsinki (Ethical Principles for Medical Research

Involving Human Subjects) and are consistent with ICH GCP applicable regulatory requirements.

Results: Patients in the study were administered the Scleroderma Health Assessment Questionnaire (SHAQ) at the time of study entry and during clinic visits following the treatment period. The study is a two-part, parallel arm design, prospective, double-blind, randomized study conducted in Australia (Flinders Medical Center). 27 patients were randomized into the first phase of the study, following which a DSMB meeting was held, which reviewed data on the primary study endpoint and key Raynaud's secondary endpoints. Data on the SHAQ was not available for the DSMB to review. The mITT population, in which any data was available post treatment was 24 patients was the analysis population for this first part of the study. The second part (i.e., part B) of the study will randomize 36 patients in a double blind, randomized, prospective, placebo controlled, crossover study. In the first part (i.e., Part A) of the study for which data is presented, patients were randomized into 6 groups, cilnidipine at two doses (10 and 20 mg) alone and in combination with tadalafil 5 mg, tadalafil 5 mg alone, and placebo. The DSMB found both doses of cilnidipine to be effective at reducing Raynaud's endpoints and increased effect with 20 mg compared to 10 mg of cilnidipine. The DSMB recommended that the study proceed into its double blind prospective cross overpowered second phase and compare cilnidipine 20 mg alone to placebo. Data from the SHAQ analysis suggests the following and is first presented in summary format and then data tables are provided.

a. Cilnidipine treatment may reduce patient-reported SSc disease disability compared to placebo
b. Cilnidipine treatment may reduce patient-reported SSc disease alternative disability compared to placebo
c. Cilnidipine treatment may reduce patient-reported SSc disease pain* compared to placebo
d. Cilnidipine treatment may reduce patient-reported SSc disease severity compared to placebo
e. Cilnidipine treatment may reduce patient-reported SSc disease skin ulcer severity rating compared to placebo
f. Cilnidipine treatment may reduce patient-reported SSc disease breathing difficulty compared to placebo
g. Cilnidipine treatment, in combination with tadalafil (a known —P glycoprotein P inhibitor that increases brain passage and central concentrations of cilnidipine (cilnidipine when given orally is 98% protein bound, largely by PGP)) may further reduce patient-reported SSc disease pain compared to placebo. SSc disease pain comprises not only pain from Raynaud's but joint pain, headache, odynophagia, back pain, pain from skin ulceration, and neuropathic pain).
   Table S4 includes results of the Scleroderma Health Assessment Questionnaire.

TABLE S4

| | Pooled | Placebo |
|---|---|---|
| Statistics | Cilnidipine (N = 7) | (N = 4) |
| Baseline | | |
| n | 7 | 4 |
| Mean (SD) | 2.700 (2.3424) | 2.000 (1.7963) |
| Median | 2.900 | 1.300 |
| Q1, Q3 | 0.400, 3.400 | 0.800, 3.200 |
| Min, Max | 0.00, 7.10 | 0.80, 4.60 |
| In-Clinic Visit (Day 12) | | |
| n | 7 | 4 |
| Mean (SD) | 1.486 (1.7743) | 2.700 (1.9511) |
| Median | 1.200 | 2.950 |
| Q1, Q3 | 0.200, 1.900 | 1.400, 4.000 |
| Min, Max | 0.00, 5.20 | 0.10, 4.80 |
| Change from Baseline | | |
| n | 7 | 4 |
| Mean (SD) | −1.214 (0.7946) | 0.700 (1.3089) |
| Median | −1.500 | 0.550 |
| Q1, Q3 | −1.800, −0.400 | −0.250, 1.650 |
| Min, Max | −1.90, 0.20 | −0.70, 2.40 |
| Percent Change from Baseline | | |
| n | 6 | 4 |
| Mean (SD) | −59.80 (25.642) | 66.71 (165.704) |
| Median | −55.86 | 27.17 |
| Q1, Q3 | −76.19, −44.12 | −41.58, 175.00 |
| Min, Max | −100.0, −26.8 | −87.5, 300.0 |

Table S4 shows that in the pooled cilnidipine patients who received either 10 mg or 20 mg of cilnidipine (n=7), there was a mean 60% decrease in patient reported disease severity on the SHAQ, versus an increase in severity in 4 placebo treated patients.
Table S5 shows a summary table comparing 20 mg of cilnidipine to placebo treated patients on the SHAQ parameters in the mITT population. Table F shows substantial benefits in the SHAQ for 20 mg treated and pooled cilnidipine patients versus placebo patients on most of the parameters measured.

TABLE S5

| | Pooled | | |
|---|---|---|---|
| | Cilnidipine | Cilnidipine | Placebo |
| PARAMETER | (n = 7) | 20 mg (n = 3) | (n = 4) |
| % change in Pain | −31% | −53% | +7% |
| % change in Standard Disability Index | −25% | −50% | −16% |
| % change in Alternative Disability Index | −13% | −42% | −13% |
| % improvement in Breathing | 41% (median) | 67% (median) | −7% (worsening) |
| % improvement in VAS Digital Ulcers | 19% | 35% | −4% (worsening) |
| % change VAS Raynauds | −17% | −49% | −42% |
| % change overall SSc Disease Severity | −60% (i.e., improvement) | −63% (i.e., improvement) | 67% (i.e., worsening) |

Pain on the SHAQ outcome measure on the first line in table S5 is a composite of all pain experienced in patients with SSc, comprising joint pain, back pain, headache, odynophagia, skin ulcer related discomfort, Raynaud's, and neuropathic pain. Aisa Pharma believes, based on the biologic attributes and actions of cilnidipine that differ from most dihydropyridine calcium channel antagonists that cilnidipine might have direct analgesic effects in certain pain conditions and in fact has been designated to receive evaluation through the NIH-NINDS Preclinical Pain Screening Platform Program to investigate the drug in multiple in vitro and in vivo preclinical models of pain that are validated and predictive of effects in man.

Analysis of data that may support an increased analgesic effect of cilnidipine on Raynaud's pain, when given in combination with tadalafil, which increases brain concentrations of cilnidipine by competitively inhibiting the protein (p-glycoprotein p) which is largely responsible for 98% protein binding of cilnidipine in plasma when the drug is given orally, inhibiting transmission across the blood-brain barrier. Given the small numbers of patients in the first part of the study, baseline pain differences vary amongst the groups, but a trend towards an increased reduction of Raynaud's VAS pain is seen in both the 10 mg cilnidipine (*) and 20 mg cilnidipine (1) groups.

Table S6 shows data that supports an increased analgesic effect of cilnidipine on Raynaud's pain.

| | | | | Percent Change |
|---|---|---|---|---|
| | Baseline | | | from Baseline |
| | Average Pain | Average Pain | Change From | (red = reduction, |
| | Score [Mean | Score [Mean | Baseline | black = increase) |
| Group (N = 24) | (SD)] | (SD)] | [Mean (SD)] | [Mean (SD)] |
| Cilnidipine 20 mg, mean (SD) (n = 3) | 1.76 (0.885) | 1.28 (1.993) | −0.47 (2.025) | −26.89 (89.725)* |
| Cilnidipine 10 mg, mean (SD) (n = 4) | 2.35 (1.758) | 2.82 (2.268) | 0.47 (0.558) | 11.76 (27.478)† |
| Tadalafil 5 mg, (n = 4) | 5.47 (3.586) | 5.84 (4.768) | 0.37 (2.219) | 17.20 (64.838) |
| Cilnidipine 10 mg + Tadalafil 5 mg, (n = 5) | 5.23 (1.445) | 2.26 (2.208) | −2.97 (1.399) | −61.84 (34.512)* |
| Cilnidipine 20 mg + Tadalafil 5 mg, (n = 4) | 4.16 (2.427) | 3.39 (3.692) | −0.77 (2.677) | −24.00 (54.180)† |
| Placebo, mean (SD) (n = 4) | 2.31 (1.623) | 1.68 (0.828) | −0.63 (1.346) | −3.92 (67.665) |

Tables S7-S14 shows listings for each parameter on the SHAQ assessment in the MITT population.
Table S7. is a SHAQ-Change from baseline and Percent change from baseline-Modified ITT Population-Part A. Standard disability index.

| | | | | Cilnidipine | Cilnidipine | |
|---|---|---|---|---|---|---|
| | Cilnidipine | Cilnidipine | Tadalafil | 10 mg + | 20 mg + | |
| | 10 mg | 20 mg | 5 mg | Tadalafil | Tadalafil | Placebo |
| Statistics | (N = 4) | (N = 3) | (N = 4) | 5 mg (N = 5) | 5 mg (N = 4) | (N = 4) |
| Baseline | | | | | | |
| n | 4 | 3 | 4 | 5 | 4 | 4 |
| Mean (SD) | 0.656 (0.7731) | 0.250 (0.2165) | 0.594 (0.4719) | 0.550 (0.4108) | 0.781 (0.8377) | 0.813 (0.6654) |
| Median | 0.563 | 0.375 | 0.625 | 0.500 | 0.750 | 0.813 |
| Q1, Q3 | 0.000, 1.313 | 0.000, 0.375 | 0.250, 0.938 | 0.250, 0.500 | 0.063, 1.500 | 0.375, 1.250 |
| Min, Max | 0.00, 1.50 | 0.00, 0.38 | 0.00, 1.13 | 0.25, 1.25 | 0.00, 1.63 | 0.00, 1.63 |
| In Clinic Visit (Day 12) | | | | | | |
| n | 4 | 3 | 4 | 8 | 4 | 4 |
| Mean (SD) | 0.656 (0.7731) | 0.125 (0.1250) | 0.531 (0.4130) | 0.391 (0.3370) | 0.714 (0.7786) | 0.719 (0.7526) |
| Median | 0.563 | 0.125 | 0.563 | 0.250 | 0.554 | 0.563 |
| Q1, Q3 | 0.000, 1.313 | 0.000, 0.250 | 0.250, 0.813 | 0.188, 0.500 | 0.125, 1.304 | 0.188, 1.250 |
| Min, Max | 0.00, 1.50 | 0.00, 0.25 | 0.00, 1.00 | 0.13, 1.13 | 0.00, 1.75 | 0.00, 1.75 |
| Change from Baseline | | | | | | |
| n | 4 | 3 | 4 | 8 | 4 | 4 |
| Mean (SD) | 0.0 (0.0000) | −0.125 (0.1250) | −0.063 (0.0722) | −0.141 (0.2449) | −0.067 (0.3063) | −0.094 (0.2772) |
| Median | 0.000 | −0.125 | −0.063 | −0.188 | 0.063 | 0.000 |
| Q1, Q3 | 0.000, 0.000 | −0.250, 0.000 | −0.125, 0.000 | −0.313, −0.063 | −0.259, 0.125 | −0.250, 0.063 |
| Min, Max | 0.00, 0.00 | −0.25, 0.00 | −0.13, 0.00 | −0.38, 0.38 | −0.52, 0.13 | −0.50, 0.13 |
| Percent Change from Baseline | | | | | | |
| n | 2 | 2 | 3 | 8 | 3 | 3 |
| Mean (SD) | 0.0 (0.000) | −50.00 (23.570) | −9.26 (8.486) | −16.88 (72.896) | 23.34 (70.153) | −16.48 (35.421) |
| Median | 0.00 | −50.00 | −11.11 | −37.50 | 7.69 | 0.00 |
| Q1, Q3 | 0.00, 0.00 | −66.67, −33.33 | −16.67, 0.00 | −62.50, −5.00 | −37.66, 100.00 | −57.14, 7.69 |
| Min, Max | 0.0, 0.0 | −66.7, −33.3 | −16.7, 0.0 | −75.0, 150.0 | −37.7, 100.0 | −57.1, 7.7 |

TABLE S8
Change from baseline and Percent change from baseline-Modified ITT Population-Part A. Parameter is the alternative disability index.

| | | | | | Cilnidipine | |
|---|---|---|---|---|---|---|
| | Cilnidipine | Cilnidipine | | Cilnidipine | 20 mg + | |
| | 10 mg | 20 mg | Tadalafil | 10 mg + Tadalafil | Tadalafil | Placebo |
| Statistics | (N = 4) | (N = 3) | 5 mg (N = 4) | 5 mg (N = 5) | 5 mg (N = 4) | (N = 4) |
| Baseline | | | | | | |
| n | 4 | 3 | 4 | 5 | 4 | 4 |
| Mean (SD) | 0.500 (0.5863) | 0.208 (0.1909) | 0.469 (0.5141) | 0.425 (0.4644) | 0.688 (0.7672) | 0.594 (0.4719) |
| Median | 0.438 | 0.250 | 0.375 | 0.250 | 0.563 | 0.625 |
| Q1, Q3 | 0.000, 1.000 | 0.000, 0.375 | 0.063, 0.875 | 0.250, 0.250 | 0.063, 1.313 | 0.250, 0.938 |
| Min, Max | 0.00, 1.13 | 0.00, 0.38 | 0.00, 1.13 | 0.13, 1.25 | 0.00, 1.63 | 0.00, 1.13 |
| In Clinic Visit (Day 12) | | | | | | |
| n | 4 | 3 | 4 | 8 | 4 | 4 |
| Mean (SD) | 0.563 (0.6575) | 0.125 (0.1250) | 0.375 (0.4787) | 0.328 (0.3532) | 0.616 (0.6594) | 0.531 (0.75242) |
| Median | 0.500 | 0.125 | 0.250 | 0.250 | 0.482 | 0.438 |
| Q1, Q3 | 0.000, 1.125 | 0.000, 0.250 | 0.000, 0.750 | 0.125, 0.375 | 0.125, 1.107 | 0.188, 0.875 |
| Min, Max | 0.00, 1.25 | 0.00, 0.25 | 0.00, 1.00 | 0.00, 1.13 | 0.00, 1.50 | 0.00, 1.25 |
| Change from Baseline | | | | | | |
| n | 4 | 3 | 4 | 8 | 4 | 4 |
| Mean (SD) | 0.063 (0.2165) | −0.083 (0.0722) | −0.094 (0.0625) | −0.031 (0.1860) | −0.071 (0.1756) | −0.063 (0.2165) |
| Median | 0.000 | −0.125 | −0.125 | −0.063 | −0.063 | 0.000 |
| Q1, Q3 | −0.063, 0.188 | −0.125, 0.000 | −0.125, −0.063 | −0.125, −0.000 | −0.205, 0.063 | −0.188, 0.063 |
| Min, Max | −0.13, 0.38 | −0.13, 0.00 | −0.13, 0.00 | −0.25, 0.38 | −0.29, 0.13 | −0.38, 0.13 |
| Percent Change from Baseline | | | | | | |
| n | 2 | 2 | 3 | 8 | 3 | 3 |
| Mean (SD) | 15.87 (38.161) | −41.67 (11.785) | −43.70 (48.956) | 11.25 (121.941) | 21.25 (68.998) | −12.96 (32.553) |
| Median | 15.87 | −41.67 | −20.00 | −5.00 | −7.69 | 0.00 |
| Q1, Q3 | −11.11, 42.86 | −50.00, −33.33 | −100.00, −11.11 | −50.00, 0.00 | −28.57, 100.00 | −50.00, 11.11 |
| Min, Max | −11.1, 42.9 | −50.0, −33.3 | −100.0, −11.1 | −100.0, 300.0 | −28.6, 100.0 | −50.0, 11.1 |

TABLE S9
Change from baseline and Percent change from baseline-Modified ITT Population-Part A. Visual Analog Scale-Pain.

| | | | | Cilnidipine | Cilnidipine | |
|---|---|---|---|---|---|---|
| | Cilnidipine | Cilnidipine | | 10 mg + | 20 mg + | |
| | 10 mg | 20 mg | Tadalafil | Tadalafil | Tadalafil | Placebo |
| Statistics | (N = 4) | (N = 3) | 5 mg (N = 4) | 5 mg (N = 5) | 5 mg (N = 4) | (N = 4) |
| Baseline | | | | | | |
| n | 4 | 3 | 3 | 5 | 3 | 4 |
| Mean (SD) | 1.975 (1.5283) | 2.167 (1.7010) | 5.967 (3.4790) | 4.320 (2.5352) | 3.167 (1.1240) | 3.325 (2.2366) |
| Median | 2.150 | 1.500 | 7.400 | 3.200 | 2.900 | 3.500 |
| Q1, Q3 | 0.850, 3.100 | 0.900, 4.100 | 2.000, 8.500 | 3.100, 5.200 | 2.200, 4.400 | 1.500, 5.150 |
| Min, Max | 0.00, 3.60 | 0.90, 4.10 | 2.00, 8.50 | 1.80, 8.30 | 2.20, 4.40 | 0.70, 5.60 |
| In Clinic Visit (Day 12) | | | | | | |
| n | 4 | 3 | 4 | 8 | 4 | 4 |
| Mean (SD) | 1.875 (1.6641) | 1.067 (0.9074) | 5.300 (4.1577) | 3.463 (1.5250) | 2.700 (1.7907) | 4.100 (3.1145) |
| Median | 1.950 | 0.700 | 5.850 | 4.050 | 2.150 | 4.450 |
| Q1, Q3 | 0.500, 3.250 | 0.400, 2.100 | 1.900, 8.700 | 2.600, 4.200 | 1.650, 3.750 | 1.600, 6.600 |
| Min, Max | 0.00, 3.60 | 0.40, 2.10 | 0.30, 9.20 | 0.50, 5.50 | 1.20, 5.30 | 0.30, 7.20 |
| Change from Baseline | | | | | | |
| n | 4 | 3 | 3 | 8 | 3 | 4 |
| Mean (SD) | −0.100 (0.4243) | −1.100 (0.7937) | 1.000 (0.4359) | −0.438 (1.3362) | −0.300 (1.3115) | 0.775 (1.2285) |
| Median | 0.000 | −0.800 | 0.800 | −0.550 | −0.100 | 0.500 |
| Q1, Q3 | −0.350, 0.150 | −2.000, −0.500 | 0.700, 1.500 | −1.200, 0.900 | −1.700, 0.900 | −0.000, 1.550 |
| Min, Max | −0.70, 0.30 | −2.00, −0.50 | 0.70, 1.50 | −2.80, 1.00 | −1.70, 0.90 | −0.40, 2.50 |
| Percent Change from Baseline | | | | | | |
| n | 3 | 3 | 3 | 8 | 3 | 4 |
| Mean (SD) | −9.88 (27.711) | −52.56 (3.454) | 31.35 (37.825) | −9.34 (36.544) | −14.24 (40.419) | 7.32 (46.946) |
| Median | 0.00 | −53.33 | 10.81 | −16.83 | −4.55 | 16.61 |
| Q1, Q3 | −41.18, 11.54 | −55.56, −48.78 | 8.24, 75.00 | −28.16, 28.13 | −58.62, 20.45 | −25.00, 39.64 |
| Min, Max | −41.2, 11.5 | −55.6, −48.8 | 8.2, 75.0 | −72.2, 31.3 | −58.6, 20.5 | −57.1, 53.2 |

TABLE S10
Change from baseline and Percent change from baseline-Modified ITT Population-Part A. Visual Analog Scale-Intestinal Problems.

| | | | | Cilnidipine | Cilnidipine | |
|---|---|---|---|---|---|---|
| | Cilnidipine | Cilnidipine | | 10 mg + | 20 mg + | |
| | 10 mg | 20 mg | Tadalafil | Tadalafil | Tadalafil | Placebo |
| Statistics | (N = 4) | (N = 3) | 5 mg (N = 4) | 5 mg (N = 5) | 5 mg (N = 4) | (N = 4) |
| Baseline | | | | | | |
| n | 4 | 3 | 4 | 5 | 4 | 3 |
| Mean (SD) | 2.975 (3.4568) | 0.833 (0.7638) | 3.150 (3.7009) | 2.020 (1.9930) | 0.925 (0.9069) | 1.100 (1.5620) |
| Median | 2.550 | 1.000 | 2.600 | 1.100 | 0.950 | 0.300 |
| Q1, Q3 | 0.050, 5.900 | 0.000, 1.500 | 0.050, 6.250 | 0.700, 2.500 | 0.150, 1.700 | 0.100, 2.900 |
| Min, Max | 0.00, 6.80 | 0.00, 1.50 | 0.00, 7.40 | 0.50, 5.30 | 0.00, 1.80 | 0.10, 2.90 |
| In Clinic Visit (Day 12) | | | | | | |
| n | 4 | 3 | 4 | 8 | 4 | 4 |
| Mean (SD) | 1.100 (1.8221) | 1.133 (1.0599) | 3.025 (4.3162) | 1.663 (1.9227) | 1.175 (2.2187) | 0.450 (0.4509) |
| Median | 0.300 | 1.300 | 1.450 | 1.350 | 0.100 | 0.300 |
| Q1, Q3 | 0.000, 2.200 | 0.000, 2.100 | 0.050, 6.000 | 0.350, 1.950 | 0.000, 2.350 | 0.150, 0.750 |
| Min, Max | 0.00, 3.80 | 0.00, 2.10 | 0.00, 9.20 | 0.00, 6.00 | 0.00, 4.50 | 0.10, 1.10 |
| Change from Baseline | | | | | | |
| n | 4 | 3 | 4 | 8 | 4 | 3 |
| Mean (SD) | −1.875 (2.1838) | 0.300 (0.3000) | −0.125 (1.6800) | −0.538 (2.1547) | −0.250 (1.8982) | −0.567 (1.0970) |
| Median | −1.550 | 0.300 | 0.000 | −0.800 | −0.150 | −0.200 |
| Q1, Q3 | −3.700, −0.050 | 0.000, 0.600 | −1.150, 0.900 | −2.000, 0.600 | −0.950, 1.450 | −1.800, 0.300 |
| Min, Max | −4.40, 0.00 | 0.00, 0.60 | −2.30, 1.80 | −3.40, 3.50 | −1.60, 2.90 | −1.80, 0.30 |
| Percent Change from Baseline | | | | | | |
| n | 3 | 2 | 3 | 8 | 3 | 3 |
| Mean (SD) | −77.37 (29.418) | 35.00 (7.071) | −6.92 (35.225) | −2.66 (99.762) | −2.55 (159.269) | 57.09 (210.380) |
| Median | −88.00 | 35.00 | 0.00 | −40.00 | −88.89 | −62.07 |
| Q1, Q3 | −100.00, −44.12 | 30.00, 40.00 | −45.10, 24.32 | −82.08, 90.00 | −100.00, 181.25 | −66.67, 300.00 |
| Min, Max | −100.0, −44.1 | 30.0, 40.0 | −45.1, 24.3 | −100.0, 142.9 | −100.0, 181.3 | −66.7, 300.0 |

TABLE S11
Change from baseline and Percent change from baseline-Modified ITT Population-Part A. Visual Analog Scale-Breathing Problems

| | | | | Cilnidipine | Cilnidipine | |
|---|---|---|---|---|---|---|
| | Cilnidipine | Cilnidipine | | 10 mg + | 20 mg + | |
| | 10 mg | 20 mg | Tadalafil | Tadalafil | Tadalafil | Placebo |
| Statistics | (N = 4) | (N = 3) | 5 mg (N = 4) | 5 mg (N = 5) | 5 mg (N = 4) | (N = 4) |
| Baseline | | | | | | |
| n | 4 | 3 | 4 | 5 | 4 | 4 |
| Mean (SD) | 1.875 (3.7500) | 1.133 (0.9018) | 3.800 (3.0144) | 1.220 (0.9550) | 1.025 (1.4930) | 0.525 (0.5852) |
| Median | 0.000 | 1.200 | 4.050 | 1.200 | 0.450 | 0.250 |
| Q1, Q3 | 0.000, 3.750 | 0.200, 2.000 | 1.550, 6.050 | 0.800, 1.500 | 0.050, 2.000 | 0.200, 0.850 |
| Min, Max | 0.00, 7.50 | 0.20, 2.00 | 0.00, 7.10 | 0.00, 2.60 | 0.00, 3.20 | 0.20, 1.40 |
| In Clinic Visit (Day 12) | | | | | | |
| n | 4 | 3 | 4 | 8 | 4 | 4 |
| Mean (SD) | 1.575 (3.1500) | 0.300 (0.2646) | 4.300 (4.8840) | 3.188 (4.2680) | 1.950 (3.3131) | 0.450 (0.3416) |
| Median | 0.000 | 0.400 | 4.000 | 1.300 | 0.450 | 0.400 |
| Q1, Q3 | 0.000, 3.150 | 0.000, 0.500 | 0.100, 8.500 | 0.350, 6.100 | 0.100, 3.800 | 0.200, 0.700 |
| Min, Max | 0.00, 6.30 | 0.00, 0.50 | 0.00, 9.20 | 0.00, 10.00 | 0.00, 6.90 | 0.10, 0.90 |
| Change from Baseline | | | | | | |
| n | 4 | 3 | 4 | 8 | 4 | 4 |
| Mean (SD) | −0.300 (0.6000) | −0.833 (1.1504) | 0.500 (2.5599) | 1.450 (4.1604) | 0.925 (1.9138) | −0.075 (0.3500) |
| Median | 0.000 | −0.800 | 1.050 | −0.400 | 0.300 | −0.050 |
| Q1, Q3 | −0.600, 0.000 | −2.000, 0.300 | −1.450, 2.450 | −0.950, 3.700 | −0.300, 2.150 | −0.350, 0.200 |
| Min, Max | −1.20, 0.00 | −2.00, 0.30 | −2.90, 2.80 | −1.90, 8.80 | −0.60, 3.70 | −0.50, 0.30 |
| Percent Change from Baseline | | | | | | |
| n | 1 | 3 | 3 | 7 | 3 | 4 |
| Mean (SD) | −16.00 | −5.56 (135.742) | −2.66 (79.815) | 108.64 (303.929) | 213.54 (347.990) | 24.40 (97.190) |
| Median | −16.00 | −66.67 | 29.58 | −26.67 | 115.63 | 7.14 |
| Q1, Q3 | −16.00, −16.00 | −100.00, 150.00 | −93.55, 56.00 | −73.08, 284.62 | −75.00, 600.00 | −51.19, 100.00 |
| Min, Max | −16.0, −16.0 | −100.0, 150.0 | −93.5, 56.0 | −100.0, 733.3 | −75.00, 600.0 | −66.7, 150.0 |

TABLE S12
Change from baseline and Percent change from baseline-Modified ITT Population-Part A. Visual Analog Scale-Raynaud's.

| | | | | Cilnidipine | Cilnidipine | |
|---|---|---|---|---|---|---|
| | Cilnidipine | Cilnidipine | | 10 mg + | 20 mg + | |
| | 10 mg | 20 mg | Tadalafil | Tadalafil | Tadalafil | Placebo |
| Statistics | (N = 4) | (N = 3) | 5 mg (N = 4) | 5 mg (N = 5) | 5 mg (N = 4) | (N = 4) |
| Baseline | | | | | | |
| n | 4 | 3 | 4 | 5 | 4 | 4 |
| Mean (SD) | 1.600 (1.9270) | 2.300 (1.3115) | 5.775 (4.0252) | 3.640 (2.9057) | 2.325 (3.9911) | 3.250 (2.3274) |
| Median | 1.000 | 2.500 | 6.400 | 2.400 | 0.500 | 2.550 |
| Q1, Q3 | 0.450, 2.750 | 0.900, 3.500 | 3.350, 8.200 | 1.400, 5.000 | 0.200, 4.450 | 1.750, 4.750 |
| Min, Max | 0.00, 4.40 | 0.90, 3.50 | 0.30, 10.00 | 1.30, 8.10 | 0.00, 8.30 | 1.30, 6.60 |
| In Clinic Visit (Day 12) | | | | | | |
| n | 4 | 3 | 4 | 8 | 4 | 4 |
| Mean (SD) | 1.925 (1.9207) | 1.033 (0.4041) | 4.950 (3.7749) | 3.400 (2.0626) | 1.500 (2.4752) | 2.750 (3.6538) |
| Median | 1.700 | 1.100 | 5.200 | 3.400 | 0.400 | 1.500 |
| Q1, Q3 | 0.400, 3.450 | 0.600, 1.400 | 2.200, 7.700 | 2.050, 4.450 | 0.150, 2.850 | 0.150, 5.350 |
| Min, Max | 0.00, 4.30 | 0.60, 1.40 | 0.20, 9.20 | 0.40, 7.00 | 0.00, 5.20 | 0.10, 7.90 |
| Change from Baseline | | | | | | |
| n | 4 | 3 | 4 | 8 | 4 | 4 |
| Mean (SD) | 0.325 (1.0468) | −1.267 (1.0599) | −0.825 (0.9674) | −0.750 (1.8150) | −0.825 (1.5262) | −0.500 (1.4306) |
| Median | 0.350 | −1.100 | −0.500 | −0.500 | −0.150 | −0.650 |
| Q1, Q3 | −0.500, 1.150 | −2.400, −0.300 | −1.500, −0.150 | −1.600, 0.150 | −1.700, 0.050 | −1.600, 0.600 |
| Min, Max | −0.90, 1.50 | −2.40, −0.30 | −2.20, −0.10 | −4.10, 2.00 | −3.10, 0.10 | −2.00, 1.30 |
| Percent Change from Baseline | | | | | | |
| n | 3 | 3 | 4 | 8 | 3 | 4 |
| Mean (SD) | 11.36 (118.770) | −48.63 (18.070) | −19.71 (16.460) | −16.67 (36.309) | −20.78 (40.151) | −41.74 (58.354) |
| Median | −2.27 | −44.00 | −20.67 | −11.00 | −37.35 | −47.18 |
| Q1, Q3 | −100.00, −136.36 | −68.57, −33.33 | −33.85, −5.56 | −47.31, 6.25 | −50.00, 25.00 | −91.61, 8.12 |
| Min, Max | −100.0, 136.4 | −68.6, −33.3 | −34.4, −3.1 | −69.2, 40.0 | −50.00, 25.0 | −92.3, 19.7 |

TABLE S13
Change from baseline and Percent change from baseline-Modified ITT Population-Part A. Finger Ulcers.

| | | | | Cilnidipine | Cilnidipine | |
|---|---|---|---|---|---|---|
| | Cilnidipine | Cilnidipine | | 10 mg + | 20 mg + | |
| | 10 mg | 20 mg | Tadalafil | Tadalafil | Tadalafil | Placebo |
| Statistics | (N = 4) | (N = 3) | 5 mg (N = 4) | 5 mg (N = 5) | 5 mg (N = 4) | (N = 4) |
| Baseline | | | | | | |
| n | 4 | 3 | 4 | 5 | 4 | 3 |
| Mean (SD) | 0.700 (0.8718) | 1.800 (2.8618) | 2.075 (4.0836) | 0.620 (0.8319) | 2.125 (4.1836) | 3.133 (4.0612) |
| Median | 0.500 | 0.300 | 0.050 | 0.200 | 0.050 | 1.200 |
| Q1, Q3 | 0.000, 1.400 | 0.000, 5.100 | 0.000, 4.150 | 0.100, 0.800 | 0.000, 4.250 | 0.400, 7.800 |
| Min, Max | 0.00, 1.80 | 0.00, 5.10 | 0.00, 8.20 | 0.00, 2.00 | 0.00, 8.40 | 0.40, 7.80 |
| In Clinic Visit (Day 12) | | | | | | |
| n | 4 | 3 | 4 | 8 | 3 | 4 |
| Mean (SD) | 0.275 (0.5500) | 0.867 (0.9609) | 2.325 (4.5836) | 0.863 (1.1070) | 1.867 (3.2332) | 2.275 (3.8922) |
| Median | 0.000 | 0.700 | 0.050 | 0.450 | 0.000 | 0.450 |
| Q1, Q3 | 0.000, 0.550 | 0.000, 1.900 | 0.000, 4.650 | 0.000, 1.500 | 0.000, 5.600 | 0.150, 4.400 |
| Min, Max | 0.00, 1.10 | 0.00, 1.90 | 0.00, 9.20 | 0.00, 3.00 | 0.00, 5.60 | 0.10, 8.10 |
| Change from Baseline | | | | | | |
| n | 4 | 3 | 4 | 8 | 3 | 3 |
| Mean (SD) | −0.425 (0.5058) | −0.933 (1.9732) | 0.250 (0.5000) | −0.275 (0.8795) | −0.967 (1.5885) | −0.167 (0.8083) |
| Median | −0.350 | 0.000 | 0.000 | −0.050 | −0.100 | 0.300 |
| Q1, Q3 | −0.850, 0.000 | −3.200, 0.400 | 0.000, 0.500 | −0.550, 0.000 | −2.800, 0.000 | −1.100, 0.300 |
| Min, Max | −1.00, 0.00 | −3.20, 0.40 | 0.00, 1.00 | −2.00, 1.00 | −2.80, 0.00 | −1.10, 0.30 |
| Percent Change from Baseline | | | | | | |
| n | 2 | 2 | 2 | 7 | 2 | 3 |
| Mean (SD) | −69.44 (43.212) | 35.29 (138.648) | 6.10 (8.623) | −30.36 (55.835) | −66.67 (47.140) | −4.27 (83.629) |
| Median | −69.44 | 35.29 | 6.10 | −12.50 | −66.67 | 3.85 |
| Q1, Q3 | −100.00, −38.89 | −62.75, 133.33 | 0.00, 12.20 | −100.00, 0.00 | −100.00, −33.33 | −91.67, 75.00 |
| Min, Max | −100.0, −38.9 | −62.7, 133.3 | 0.0, 12.2 | −100.0, 50.0 | −100.0, −33.3 | −91.7, 75.0 |

TABLE S14
Change from baseline and Percent change from baseline-Modified ITT Population-Part A. Overall Change in Severity.

| | | | | Cilnidipine | Cilnidipine | |
|---|---|---|---|---|---|---|
| | Cilnidipine | Cilnidipine | | 10 mg + | 20 mg + | |
| | 10 mg | 20 mg | Tadalafil | Tadalafil | Tadalafil | Placebo |
| Statistics | (N = 4) | (N = 3) | 5 mg (N = 4) | 5 mg (N = 5) | 5 mg (N = 4) | (N = 4) |
| Baseline | | | | | | |
| n | 4 | 3 | 4 | 5 | 4 | 4 |
| Mean (SD) | 2.725 (3.2877) | 2.667 (0.4933) | 4.975 (3.3837) | 3.300 (2.3152) | 3.600 (3.2321) | 2.000 (1.7963) |
| Median | 1.900 | 2.900 | 6.000 | 1.900 | 2.550 | 1.300 |
| Q1, Q3 | 0.200, 5.250 | 2.100, 3.000 | 2.800, 7.150 | 1.900, 4.700 | 1.250, 5.950 | 0.800, 3.200 |
| Min, Max | 0.00, 7.10 | 2.10, 3.00 | 0.10, 7.80 | 1.30, 6.70 | 1.20, 8.10 | 0.80, 4.60 |
| In Clinic Visit (Day 12) | | | | | | |
| n | 4 | 3 | 4 | 8 | 4 | 4 |
| Mean (SD) | 1.825 (2.4061) | 1.033 (0.4726) | 5.600 (4.2528) | 2.975 (1.8367) | 2.750 (3.0957) | 2.700 (1.9511) |
| Median | 1.050 | 1.200 | 6.550 | 3.300 | 1.500 | 2.950 |
| Q1, Q3 | 0.100, 3.550 | 0.500, 1.400 | 2.200, 9.000 | 1.150, 4.050 | 0.800, 4.700 | 1.400, 4.000 |
| Min, Max | 0.00, 5.20 | 0.50, 1.40 | 0.20, 9.10 | 0.80, 6.00 | 0.70, 7.30 | 0.10, 4.80 |
| Change from Baseline | | | | | | |
| n | 4 | 3 | 4 | 8 | 4 | 4 |
| Mean (SD) | −0.900 (0.9695) | −1.633, (0.1528 | 0.625, (1.5903) | −0.850 (1.4948) | −0.850 (0.5916) | 0.700 (1.3089) |
| Median | −0.950 | −1.600 | 0.700 | −0.700 | −0.650 | 0.550 |
| Q1, Q3 | −1.700, −0.100 | −1.800, −1.500 | −0.600, 1.850 | −1.500, −0.450 | −1.250, −0.450 | −0.250, 1.650 |
| Min, Max | −1.90, 0.20 | −1.80, −1.50 | −1.30, 2.40 | −3.40, 1.90 | −1.70, −0.40 | −0.70, 2.40 |
| Percent Change from Baseline | | | | | | |
| n | 3 | 3 | 4 | 8 | 4 | 4 |
| Mean (SD) | −56.96 (38.271) | −62.64 (12.445) | 32.49 (51.568) | −16.48 (52.198) | −31.76 (15.773) | 66.71 (165.704) |
| Median | −44.12 | −60.00 | 26.790 | −18.99 | −36.22 | 27.17 |
| Q1, Q3 | −100.00, −26.76 | −76.19, −51.72 | −3.48, 68.46 | −49.16, −11.61 | −43.20, −20.32 | −41.58, 175.00 |
| Min, Max | −100.0, −26.8 | −76.2, −51.7 | −23.6, 100.0 | −72.3, 100.0 | −44.7, −9.9 | −87.5, 300.0 |

Table S15 shows the SHAQ parameter differences between Pooled cilnidipine (n) and placebo (n). Results with a (*) show a favorable treatment effect for cilnidipine.

| | Pooled | Placebo |
|---|---|---|
| SHAQ PARAMETER | cilnidipine (n) | (n) |
| % from baseline, standard | −25, −16.7 (4)* | −16.5, 0 (3) |
| disability mean, median | | |
| % from baseline, alternative | −12.9, −22.2 (4)* | −13, 0 (3) |
| disability mean, median | | |
| % from baseline, VAS Pain mean, | −31.2, −45 (6)* | 7.3, 16.6 (3) |
| median | | |
| % from baseline, Intestinal | −32.4, −44.1 (5)* | 57, −62 (3) |
| Problems mean, median | | |
| % from baseline, VAS Breathing | −8.2, −41.3 (4)* | 24.4, 7.1 (4) |
| mean, median | | |
| % from baseline, VAS Raynaud's | −18.6, −38.7 (6) | −41.8, −47.2 (4) |
| mean, median | | |
| % from baseline, VAS Finger | −17.1, −50.9 (4)* | −4.3, 3.9 (3) |
| Ulcers mean, median | | |
| % from baseline, VAS Overall | −59.8, −55.9 (6)* | 66.7, 27 (4) |
| Disease Severity mean, median | | |

Table S16 shows difference for pooled cilnidipine patients compared to placebo on the SHAQ rating of overall disease severity is significant (p=0.01).

Scleroderma health assessment questionnaire (SHAQ)

| | | | Wilcoxon | T test |
|---|---|---|---|---|
| Parameter | 20 mg | placebo | p value | p value |
| VAS-Pain | −1.1 (0.79) | 0.78 (1.23) | 0.200 | 0.136 |
| VAS- Breathing problems Change | −0.83 (1.15) | −0.075 (0.35) | 0.943 | 0.540 |
| VAS- Raynaud's Change | −1.27 (1.06) | −0.5 (1.43) | 0.686 | 0.436 |
| VAS- overall disease severity Change | −1.63 (0.15) | 0.7 (1.31) | 0.029 | 0.0115 |

Other results

| | | Cilnidipine | |
|---|---|---|---|
| | | (20 mg) | Rirash 2017 |
| Parameter | Placebo(n = 4) | **(n-3) | metanalysis^{†} |
| Mean weekly attacks at baseline | 16.7 | 12.7 | 16.4 (n = 117 for 2° RP) |
| Mean % reduction of weekly attacks at baseline | 18.9* | 43 | 24% (n = 117 for 2° RP) |
| Mean severity at baseline | 3.3 | 3.7 | Unknown |
| Mean % reduction in severity | −16.2 | 35.5 | 10.2(n = 138 for 2° RP) |
| Mean duration of attack at baseline | 164.2 | 19.4 | Unknown |
| Mean % reduction in duration | 12.3 | 20.0 | 6(n = 138) |
| Mean pain level at baseline | 2.3 | 2.3 | Unknown |
| Mean % reduction in pain | −3.9 | −11.8 | Ns |
| Mean RCS at baseline | 2.6 | 3.5 | Unknown |
| Mean % reduction in RCS | 5.1 | 27.9 | 9(n = 192) |

^{†}Rirash, F. et.al.; Cochrane Syst Rev. 2017 Dec. 13; 12 (12)

Table S17 shows additional results.

| | Cilnidipine 20 mg | |
|---|---|---|
| Parameter | (n = 3) | Placebo (n = 4) |
| Standard disability Index | 50% improvement | 11% improvement |
| Alternative disability index | 42% improvement | 13% improvement |
| VAS- PAIN | 53% improvement* | 23% worse** |
| VAS - Breathing problems | 75% improvement* | 24% improvement |
| VAS - Raynaud's | 55% improvement | 16% improvemnt |
| VAS Overall Disease Severity | 63% improvement | 35% worse |

*Baseline was 2.17 on a 0-10 VAS
**Baseline was 3.3 on a 0-10 VAS
***Baseline was 1.13 on a 0-10 VAS

Table S18 provides a summary of data:

| | No treatment | Grade 1 Adverse | |
|---|---|---|---|
| Safety | D/Cs | Events (AEs) only | Efficacy Seen |
| Dose | 20 mg dose | Worst AE in | 20 mg dose group |
| response | superior to | tadalafil | shows best overall |
| seen | all groups > | monotherapy | response |
| | Part B | group | |
| Tadalafil | 17% adverse | Study conduct | Attacks reduced |
| 10 mg | effects in any | good | 43%_vs 23% with |
| helps | Profervia | | standard calcium |
| | dose | | channel blockers |

Table S19 shows a dose response comparison between cilnidipine 10 mg and 20 mg.

| | Pooled | | |
|---|---|---|---|
| | Cilnidipine | Cilnidipine | Placebo |
| PARAMETER | (n = 7) | 20 mg (n = 3) | (n = 4) |
| Weekly attack | −41% | −43% | −19% |
| frequency | | | |
| If baseline | −27% | −42% | −18% |
| RCS <3.5 | | | |
| Duration | 20% | −20% | −12% |
| Severity | −16% | −36% | 16% |
| Daily RCS Change | 12% (n = 3) | −28% | −5% |
| Average Raynaud's | −27% | 12% | 4% |
| Pain | | | |
| Safety | 1AE (in combo | 1AE (HA-mild; | 1AE (Increase |
| | with T); no | 1 withdrawal | in GERD) |
| | withdrawals | (n = 8) | |
| | (n = 9) | | |
| Subgroup attack | <10 | <10 | <10 |
| frequency | baseline −55% | baseline −29% | baseline 43% |
| | (n = 2) | (n = 2) | (n = 1) |
| | >10 | >10 | >10 |
| | baseline −28% | baseline −50% | baseline −39% |
| | (n = 2) | (n = 2) | (n = 2) |
| Subgroup | <3.5 | <3.5 | <3.5 |
| Baseline RCS | baseline −27% | baseline −42% | baseline −18% |
| | (n = 3) | (n = 1) | (n = 3) |
| | 3.5-7.5 | >3.5-7.5 | 3.5-7.5 |
| | baseline −86% | baseline −43% | baseline −32% |
| | (n = 1) | (n = 2) | (n = 3) |

This evaluation study demonstrated successful implementation of the study processes, as indicated by the absence of Serious Adverse Events (SAEs), unblinding, or treatment discontinuations due to drug-related effects. Furthermore, only Grade 1 Adverse Events (AEs) were reported for patients treated with cilnidipine. Both cilnidipine doses, 10 mg and 20 mg, were well tolerated when administered alone or in combination with tadalafil. Notably, the 20 mg dose exhibited a greater overall effect compared to the 10 mg dose. The addition of tadalafil to cilnidipine demonstrated a more pronounced effect with the 10 mg dose compared to the 20 mg dose of cilnidipine alone. In comparison to an imputed historical comparator encompassing all Calcium Channel Blocker (CCB) trials for Systemic Sclerosis-Related Raynaud's Phenomenon (SSc-RP), cilnidipine exhibited improved safety with a lower incidence of Adverse Events (17% AE versus 43% AE, p=0.0247). Cilnidipine demonstrated an improvement in the overall severity of Systemic Sclerosis (SSc) as measured by the Scleroderma Health Assessment Questionnaire (SHAD) when compared to the placebo group (p=0.01). Specifically, in the group receiving cilnidipine 20 mg (n=3), there was an improvement in the SHAQ scales of disability, pain, skin ulcers, Raynaud's phenomenon, and breathing difficulty compared to the placebo group (n=4). Furthermore, the addition of tadalafil, which enhances the brain concentration of cilnidipine by inhibiting plasma proteins that bind the drug, resulted in improved pain relief related to all causes of SSc when compared to cilnidipine alone.

### CONCLUSION

In this study, cilnidipine demonstrated good tolerability among patients, with an overall adverse event rate of 17% observed in all patients who received any dose of cilnidipine (n=17). Importantly, only mild (Grade 1) adverse events occurred, and no treatment discontinuations due to intolerance were reported. A dose response was evident between cilnidipine 10 mg and 20 mg, with the higher dose showing a greater effect. Regarding the primary endpoint, cilnidipine 20 mg led to a significant reduction of 43% in weekly attack frequency, compared to 19% in patients treated with placebo. This reduction surpasses the clinically meaningful threshold of 25%. Comparatively, in this study, cilnidipine appeared to be safer and more effective than a large published meta-analysis of Calcium Channel Blocker (CCB) use in Raynaud's (both primary and secondary). Moreover, cilnidipine 20 mg exhibited a more substantial impact on alleviating the underlying burdens and manifestations of systemic sclerosis (SSc) when compared to placebo. In summary, cilnidipine holds promise as a well-tolerated and effective treatment option for patients with secondary Raynaud's primarily due to SSc.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating eye pain in a subject in need thereof, the method comprising administering a therapeutically effective amount of cilnidipine or a pharmaceutically acceptable salt thereof to an eye of the subject.

2. The method of claim 1, wherein the pain is neuropathic pain.

3. A method of treating an eye disease or disorder in a subject in need thereof, the method comprising administering a therapeutically effective amount of cilnidipine or a pharmaceutically acceptable salt thereof to an eye of the subject, wherein the disease or disorder is selected from allergies, blepharitis, chalazion, complication of eye surgery, contact lens problem, corneal abrasion, corneal herpetic infections, dry eyes, dry eye disease, ectropion, entropion eyelid infection, foreign object in the eye, glaucoma, hyphema, injury to the eye, iritis, keratitis, microvascular cranial nerve palsy, optic neuritis, pink eye, scleritis, stye, uveitis, or any combination thereof.

4. The method of claim 3, wherein the disease or disorder is dry eye disease.

5. The method of claim 3, wherein the disease or disorder is uveitis.

6. The method of claim 3, wherein the disease or disorder is optic neuritis.

7. The method of claim 3, wherein the disease or disorder is microvascular cranial nerve palsy.

8. The method of claim 3, wherein the disease or disorder is hyphema.

9. The method of claim 3, wherein the disease or disorder is glaucoma.

10.-35. (canceled)

36. The method of claim 3, wherein the subject has pain in an eye, and wherein after administering the cilnidipine or pharmaceutically acceptable salt thereof, the eye pain is reduced.

37. The method of claim 36, wherein the pain is neuropathic pain.

38. The method of claim 36, wherein the reduction in eye pain is assessed or measured by the eye-wiping test, the Ocular Pain Assessment Scale (OPAS), the Schirmer Tear Test (STT), or applanation tonometry.

39. The method of claim 3, wherein an at least 2% reduction of intraocular pressure in the eye of the subject is measured by applanation tonometry after administering the cilnidipine or pharmaceutically acceptable salt thereof.

40. (canceled)

41. The method of claim 3, wherein after administration of the cilnidipine or pharmaceutically acceptable salt thereof to the subject, substantially no anesthesia of the eye is detected in the subject according to the blink reflex test.

42. The method of claim 3, wherein after administration of the cilnidipine or pharmaceutically acceptable salt thereof to the subject, the visual acuity of the subject improves.

43. The method of claim 42, wherein improvement in the visual acuity of the subject is assessed by an improvement in a ETDRS letter chart assessment.

44.-47. (canceled)

48. The method of claim 3, wherein the dosage form of the cilnidipine comprises a concentration of from about 0.01% to about 10% by weight of cilnidipine.

49.-51. (canceled)

52. The method of claim 3, wherein the cilnidipine or pharmaceutically acceptable salt thereof is administered topically.

53. The method of claim 3, wherein the cilnidipine or pharmaceutically acceptable salt thereof is administered intraocularly.

54. The method of claim 3, wherein the cilnidipine or pharmaceutically acceptable salt thereof is administered as a solution or suspension.

55.-74. (canceled)
